(19) United States
(12) Patent Application Publication
(10) Pub. No.: US 2024/0287484 A1
(43) Pub. Date: Aug. 29, 2024

(54) SYSTEMS, COMPOSITIONS, AND METHODS INVOLVING RETROTRANSPOSONS AND FUNCTIONAL FRAGMENTS THEREOF

(71) Applicant: Metagenomi, Inc., Emeryville, CA (US)

(72) Inventors: Brian C. THOMAS, Berkeley, CA (US); Christopher BROWN, Albany, CA (US); Daniela S.A. GOLTSMAN, Oakland, CA (US); Sarah LAPERRIERE, Berkeley, CA (US); Cindy CASTELLE, San Francisco, CA (US); Lisa ALEXANDER, Albany, CA (US); Mary Kaitlyn Chiu, San Francisco, CA (US); Morayma TEMOCHE-DIAZ, Oakland, CA (US); Anu THOMAS, Albany, CA (US)

(21) Appl. No.: 18/598,627

(22) Filed: Mar. 7, 2024

Related U.S. Application Data
(63) Continuation of application No. PCT/US2022/076061, filed on Sep. 7, 2022.
(60) Provisional application No. 63/241,943, filed on Sep. 8, 2021.

(51) Int. Cl. C12N 9/12; C12N 9/22; C12N 15/10

(52) U.S. Cl. CPC C12N 9/1276; C12N 9/22; C12N 15/1096; C12Y 207/07049

(57) ABSTRACT

The present disclosure provides systems and methods for transposing a cargo nucleotide sequence to a target nucleic acid site. These systems and methods can comprise a nucleic acid comprising the cargo nucleotide sequence, wherein the cargo nucleotide sequence is configured to interact with a retrotransposase, and the retrotransposase, wherein said retrotransposase is configured to transpose the cargo nucleotide sequence to the target nucleic acid site. The systems and methods can also involve use of functional fragments of retrotransposases.

### CROSS-REFERENCE

This application is a continuation of International Application No. PCT/US2022/076061, entitled “SYSTEMS, COMPOSITIONS, AND METHODS INVOLVING RETROTRANSPOSONS AND FUNCTIONAL FRAGMENTS THEREOF”, filed on Sep. 7, 2022, which claims the benefit of U.S. Provisional Application No. 63/241,943, entitled “SYSTEMS AND METHODS FOR TRANSPOSING CARGO NUCLEOTIDE SEQUENCES”, filed on Sep. 8, 2021, which is incorporated by reference herein in its entirety.

### BACKGROUND

Transposable elements are movable DNA sequences which play a crucial role in gene function and evolution. While transposable elements are found in nearly all forms of life, their prevalence varies among organisms, with a large proportion of the eukaryotic genome encoding for transposable elements (at least 45% in humans).

### SEQUENCE LISTING

The instant application contains a Sequence Listing which has been submitted electronically in XML format and is hereby incorporated by reference in its entirety. Said XML copy, created on May 13, 2024, is named 55921-734301.xml and is 1,700,073 bytes in size.

### SUMMARY

While the foundational research on transposable elements was conducted in the 1940s, their potential utility in DNA manipulation and gene editing applications has only been recognized in recent years.

In some aspects, the present disclosure provides for an engineered retrotransposase system, comprising: (a) an RNA comprising a heterologous engineered cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase comprises a reverse transcriptase (RT) domain, an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 1-29 or 393-401, or a variant thereof. In some embodiments, the retrotransposase further comprises any of the Zn-binding ribbon motifs of any one of SEQ ID NOs: 1-29 or 393-401, or a variant thereof. In some embodiments, the retrotransposase further comprises a sequence having at least 80% sequence identity to any one of SEQ ID NOs: 1-29 or 393-401, or a variant thereof. In some embodiments, wherein the retrotransposase further comprises a conserved catalytic D, QG, [Y/F]XDD, or LG motif relative to any of the sequences in FIG. 2A. In some embodiments, the retrotransposase further comprises a conserved CX_{[2-3]}C Zn finger motif relative to any of the sequences in FIG. 2B. In some embodiments, the retrotransposase comprises a sequence having at least 80% sequence identity to any one of SEQ ID NOs: 3, 6, 7, 8, 14, or 402, or a variant thereof. In some embodiments, the system further comprises: (c) a double-stranded DNA sequence comprising the target nucleic acid locus. In some embodiments, the double-stranded DNA sequence comprises a 5′ recognition sequence and a 3′ recognition sequence configured to interact with the retrotransposase, wherein the 5′ recognition sequence comprises a GG nucleotide sequence and the 3′ recognition sequence comprises a TGAC nucleotide sequence. In some embodiments, the RNA is an in vitro transcribed RNA. In some embodiments, the RNA comprises a sequence 5′ to the cargo sequence or a sequence 3′ to the cargo sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RNA cognate of any one of SEQ ID NOs: 761-798, a complement thereof, or a reverse complement thereof. In some embodiments, the RNA comprises a sequence encoding the retrotransposase. In some embodiments, the heterologous engineered cargo nucleotide sequence comprises an expression cassette.

In some embodiments, the present disclosure provides for an engineered DNA sequence, comprising: (a) a 5′ sequence capable of encoding an RNA sequence configured to interact with a retrotransposase; (b) a heterologous cargo sequence; (c) a sequence encoding a retrotransposase configured to interact with an RNA cognate of the 5′ sequence, wherein the retrotransposase comprises a reverse transcriptase (RT) domain or an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a RT or endonuclease domain of any one of SEQ ID NOs: 1-29 or 393-401, or a variant thereof; and (d) a 3′ sequence capable of encoding an RNA sequence configured to interact with the retrotransposase. In some embodiments, the retrotransposase further comprises any of the Zn-binding ribbon motifs of any one of SEQ ID NOs: 1-29 or 393-401, or a variant thereof. In some embodiments, the retrotransposase further comprises a sequence having at least 80% sequence identity to any one of SEQ ID NOs: 1-29 or 393-401, or a variant thereof. In some embodiments, the retrotransposase further comprises a conserved catalytic D, QG, [Y/F]XDD or LG motif relative to any of the sequences in FIG. 2A. In some embodiments, the retrotransposase further comprises a conserved CX_{[2-3]}C Zn finger motif relative to any of the sequences in FIG. 2B. In some embodiments, the retrotransposase comprises a sequence having at least 80% sequence identity to any one of SEQ ID NOs: 3, 6, 7, 8, 14, or 402, or a variant thereof. In some embodiments, the 5′ sequence or the 3′ sequence comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RNA cognate of any one of SEQ ID NOs: 761-798, a complement thereof, or a reverse complement thereof.

In some aspects, the present disclosure provides for a method for synthesizing complementary DNA (cDNA), comprising: (a) providing an RNA molecule as a template for cDNA synthesis, (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 1-29, 393-401, or 427-439, or a variant thereof. In some embodiments, the reverse transcriptase comprises a sequence having at least 80% sequence identity to any one of SEQ ID NOs: 799-894 or 427-439, or a variant thereof. In some embodiments, the primer oligonucleotide comprises an oligo(dT) sequence or a degenerate sequence of at least six oligonucleotides. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a protein comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 1-29, 393-401, or 427-439, or a variant thereof, wherein the sequence is fused N- or C-terminally to a non-retrotransposase domain or an affinity tag. In some embodiments, the reverse transcriptase domain comprises a sequence having at least 80% sequence identity to any one of SEQ ID NOs: 799-894, 427-439, or a variant thereof. In some embodiments, the non-retrotransposase domain is an RNA-binding protein domain. In some embodiments, the RNA binding protein domain comprises a bacteriophage MS2 coat protein (MCP) domain

In some aspects, the present disclosure provides for a nucleic acid encoding any of the proteins described herein.

In some aspects, the present disclosure provides for a nucleic acid encoding an open reading frame, wherein the open reading frame encodes an RT or endonuclease domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 1-29, 393-401, or 427-439, or a variant thereof, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes a retrotransposase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 1-29, 393-401, or 427-439, or a variant thereof.

In some embodiments, the present disclosure provides for an engineered retrotransposase system, comprising: (a) an RNA comprising a heterologous engineered cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase comprises a reverse transcriptase (RT) domain or an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a RT or endonuclease domain of SEQ ID NO: 402 or 895, or a variant thereof. In some embodiments, the retrotransposase further comprises any of the Zn-binding ribbon motifs of SEQ ID NO: 402 or 895. In some embodiments, the retrotransposase further comprises a sequence having at least 80% sequence identity to SEQ ID NO: 402 or 895, or a variant thereof. In some embodiments, the retrotransposase further comprises a conserved catalytic D, QG, [Y/F]XDD, or LG motif of SEQ ID NO: 402 or 895. In some embodiments, the retrotransposase further comprises a conserved CX_{[2-3]}C Zn finger motif of SEQ ID NO: 402 or 895. In some embodiments, the system further comprises: (c) a double-stranded DNA sequence comprising the target locus. In some embodiments, the RNA is an in vitro transcribed RNA. In some embodiments, the RNA comprises a sequence encoding the retrotransposase.

In some aspects, the present disclosure provides for an engineered DNA sequence, comprising: (a) a 5′ sequence capable of encoding an RNA sequence configured to interact with a retrotransposase; (b) a heterologous cargo sequence; (c) a sequence encoding a retrotransposase configured to interact with an RNA cognate of the 5′ sequence, wherein the retrotransposase comprises a reverse transcriptase (RT) domain, an endonuclease domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a RT or endonuclease domain of SEQ ID NO: 402 or 895, or a variant thereof; and (d) a 3′ sequence capable of encoding an RNA sequence configured to interact with the retrotransposase. In some embodiments, the retrotransposase further comprises any of the Zn-binding ribbon motifs of SEQ ID NO: 402 or 895. In some embodiments, the retrotransposase further comprises a sequence having at least 80% sequence identity to SEQ ID NO: 402 or 895, or a variant thereof. In some embodiments, the retrotransposase further comprises a conserved catalytic D, QG, [Y/F]XDD or LG motif of SEQ ID NO: 402 or 895. In some embodiments, the retrotransposase further comprises a conserved CX_{[2-3]}C Zn finger motif of SEQ ID NO: 402 or 895.

In some aspects, the present disclosure provides for a method for synthesizing complementary DNA (cDNA), comprising: (a) providing an RNA molecule as a template for cDNA synthesis, (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of SEQ ID NO: 402 or 895, or a variant thereof. In some embodiments, the reverse transcriptase comprises a sequence having at least 80% sequence identity to SEQ ID NO: 402 or 895, or a variant thereof. In some embodiments, the primer oligonucleotide comprises an oligo(dT) sequence or a degenerate sequence of at least six oligonucleotides. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a protein comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of SEQ ID NO: 402 or 895, or a variant thereof, wherein the sequence is fused N- or C-terminally to a non-retrotransposase domain or an affinity tag. In some embodiments, the reverse transcriptase domain comprises a sequence having at least 80% sequence identity to SEQ ID NO: 402 or 895, or a variant thereof. In some embodiments, the non-retrotransposase domain is an RNA-binding protein domain. In some embodiments, the RNA binding protein domain comprises a bacteriophage MS2 coat protein (MCP) domain.

In some aspects, the present disclosure provides for a nucleic acid encoding an open reading frame, wherein the open reading frame encodes an RT or endonuclease domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of SEQ ID NO: 402 or 895, or a variant thereof, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes a retrotransposase comprising a sequence having at least 80% sequence identity to SEQ ID NO: 402 or 895, or a variant thereof.

In some aspects, the present disclosure provides for a method for synthesizing complementary DNA (cDNA), comprising: (a) providing an RNA molecule as a template for cDNA synthesis, (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 555-728, or a variant thereof. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, 608, 561, 562, 564, 565, 568, 571, 573, 576-579, 583, 590, 591, 594, 598, 601, 606, 607, or a variant thereof. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, 608, or a variant thereof. In some embodiments, the primer oligonucleotide comprises an oligo(dT) sequence or a degenerate sequence of at least six oligonucleotides. In some embodiments, the primer oligonucleotide comprises at least one phosphorothioate linkage. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a protein comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 555-728, or a variant thereof, wherein the sequence is fused N- or C-terminally to a non-retrotransposase domain or an affinity tag. In some embodiments, the reverse transcriptase domain comprises a sequence having at least 80% sequence identity to any one of SEQ ID NOs: 555-560, 563, 564, 566, 567, 569, 572, 574, 580-582, 584-588, 592, 593, 596, 602, 604, 605, 608, 561, 562, 564, 565, 568, 571, 573, 576-579, 583, 590, 591, 594, 598, 601, 606, 607, or a variant thereof. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, 608, or a variant thereof. In some embodiments, the non-retrotransposase domain is an RNA-binding protein domain. In some embodiments, the RNA binding protein domain comprises a bacteriophage MS2 coat protein (MCP) domain. In some embodiments, the protein comprises a sequence having at least 80% sequence identity to any one of SEQ ID NOs: 30-32, 40-50, 740-756, 757-760, or a variant thereof. In some embodiments, the reverse transcriptase domain comprises a sequence having at least 80% sequence identity to any one of SEQ ID NOs: 555-558, 561-567, 569, 570, 575, or a variant thereof.

In some aspects, the present disclosure provides for a nucleic acid encoding an open reading frame, wherein the open reading frame encodes an RT or endonuclease domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT or endonuclease domain of any one of SEQ ID NOs: 555-728, or a variant thereof, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes a retrotransposase comprising a sequence having at least 80% sequence identity to an RT or endonuclease domain of any one of SEQ ID NOs: 555-560, 563, 564, 566, 567, 569, 572, 574, 580-582, 584-588, 592, 593, 596, 602, 604, 605, 608, 561, 562, 564, 565, 568, 571, 573, 576-579, 583, 590, 591, 594, 598, 601, 606, 607, or a variant thereof. In some embodiments, the reverse transcriptase comprises a sequence having at least 80% sequence identity to any one of SEQ ID NOs: 555-560, 563, 564, 566, 567, 569, 572, 574, 580-582, 584-588, 592, 593, 596, 602, 604, 605, 608, or a variant thereof.

In some aspects, the present disclosure provides for a nucleic acid comprising a sequence comprising an open reading frame (ORF) comprising a sequence encoding a reverse transcriptase domain or a maturase domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain or a maturase domain of any one of SEQ ID NOs: 729-733, or a variant thereof, wherein: (a) the open reading frame is optimized for expression in an organism and the organism is different to the origin of the RT or endonuclease domain; or (b) the ORF comprises a sequence encoding an affinity tag. In some embodiments, the ORF encodes a protein having at least 80% sequence identity to any one of SEQ ID NOs: 729-733, or a variant thereof. In some embodiments, the ORF is optimized for expression in the bacterial organism or wherein the organism is E. coli. In some embodiments, the ORF is optimized for expression in a mammalian organism or wherein the organism is a primate organism. In some embodiments, the primate organism is H. sapiens. In some embodiments, the ORF comprises an affinity tag operably linked to the sequence encoding the reverse transcriptase domain or the maturase domain, wherein the ORF has at least 80% sequence identity to any one of SEQ ID NOs: 298-302. In some embodiments, the ORF comprises a sequence having at least 80% sequence identity to any one of SEQ ID NOs: 303-307. In some embodiments, the reverse transcriptase domain or the maturase domain comprises a conserved Y[I/L]DD active site motif of any one of SEQ ID NOs: 729-733.

In some aspects, the present disclosure provides for a method for synthesizing complementary DNA (cDNA), comprising: (a) providing an RNA molecule as a template for cDNA synthesis; (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 440-554, or a variant thereof. In some embodiments, the reverse transcriptase comprises a sequence having at least 80% sequence identity to any one of SEQ ID NOs: 518-522, 524-527, and 529-532, or a variant thereof. In some embodiments, the reverse transcriptase comprises a sequence having at least 80% sequence identity to any one of SEQ ID NOs: 526 or a variant thereof. In some embodiments, the primer oligonucleotide comprises an oligo(dT) sequence or a degenerate sequence of at least six oligonucleotides. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a protein comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 440-554, or a variant thereof, wherein the sequence is fused N- or C-terminally to a non-retrotransposase domain or an affinity tag. In some embodiments, the reverse transcriptase domain comprises a sequence having at least 80% sequence identity to any one of SEQ ID NOs: 518-522, 524-527, and 529-532, or a variant thereof. In some embodiments, the reverse transcriptase comprises a sequence having at least 80% sequence identity to SEQ ID NO: 526, or a variant thereof. In some embodiments, the non-retrotransposase domain is an RNA-binding protein domain. In some embodiments, the RNA binding protein domain comprises a bacteriophage MS2 coat protein (MCP) domain. In some embodiments, the sequence is fused N- or C-terminally to an affinity tag.

In some aspects, the present disclosure provides for a nucleic acid encoding an open reading frame, wherein the open reading frame encodes an RT domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT domain of any one of SEQ ID NOs: 440-554, or a variant thereof, wherein: (a) the open reading frame is optimized for expression in an organism and the organism is different to the origin of the RT or endonuclease domain; or (b) the ORF comprises a sequence encoding an affinity tag. In some embodiments, the nucleic acid further encodes an RT having at least 80% sequence identity to any one of SEQ ID NOs: 518-522, 524-527, and 529-532, or a variant thereof. In some embodiments, the reverse transcriptase comprises a sequence having at least 80% sequence identity to SEQ ID NOs: 526, or a variant thereof. In some embodiments, the open reading frame comprises a sequence having at least 80% sequence identity to any one of SEQ ID NOs: 356-373.

In some aspects, the present disclosure provides for a method for synthesizing complementary DNA (cDNA), comprising: (a) providing an RNA molecule as a template for cDNA synthesis; (b) providing a primer oligonucleotide to initiate cDNA synthesis from the RNA molecule; and (c) synthesizing cDNA initiated by the primer oligonucleotide from the template using a reverse transcriptase comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 609-610, 611-615, 616-617, 618-622, 623, 624-626, 627-673, or a variant thereof. In some embodiments, the reverse transcriptase domain comprises a conserved xxDD, [F/Y]XDD, NAxxH, or VTG motif of any one of SEQ ID NOs: 609-610, 611-615, 616-617, 618-622, 623, 624-626, or 627-673. In some embodiments, the reverse transcriptase comprises a sequence having at least 80% sequence identity to any one of SEQ ID NOs: 612-613, 616-619, 622, 624, 627-630, 633, or a variant thereof. In some embodiments, the primer oligonucleotide comprises an oligo(dT) sequence or a degenerate sequence of at least six oligonucleotides. In some embodiments, the primer oligonucleotide comprises at least six consecutive nucleotides having at least 80% sequence identity to any one of SEQ ID NOs: 340-341, 342-344, 345-346, 347-351, 352, or 353-355. In some embodiments, the synthesizing cDNA comprises incubating the template RNA molecule, the primer oligonucleotide, and the reverse transcriptase in a reaction mixture under conditions suitable for extension of a DNA sequence from the RNA template. In some embodiments, the reaction mixture further comprises dNTPs, a reaction buffer, divalent metal ions, Mg²⁺, or Mn²⁺.

In some aspects, the present disclosure provides for a protein comprising a reverse transcriptase domain comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a reverse transcriptase domain of any one of SEQ ID NOs: 609-610, 611-615, 616-617, 618-622, 623, 624-626, 627-673, or a variant thereof, wherein the sequence is fused N- or C-terminally to a non-retrotransposase domain or affinity tag. In some embodiments, the reverse transcriptase domain comprises a conserved xxDD, [F/Y]XDD, NAxxH, or VTG motif of any one of SEQ ID NOs: 609-610, 611-615, 616-617, 618-622, 623, 624-626, or 627-673. In some embodiments, the reverse transcriptase domain comprises a sequence having at least 80% sequence identity to any one of SEQ ID NOs: 612-613, 616-619, 622, 624, 627-630, 633, or a variant thereof. In some embodiments, the non-retrotransposase domain is an RNA-binding protein domain. In some embodiments, the RNA binding protein domain comprises a bacteriophage MS2 coat protein (MCP) domain. In some embodiments, the sequence is fused N- or C-terminally to an affinity tag.

In some aspects, the present disclosure provides for a nucleic acid encoding an open reading frame (ORF) optimized for expression in an organism, wherein the open reading frame encodes an RT domain having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an RT domain of any one of SEQ ID NOs: 609-610, 611-615, 616-617, 618-622, 623, 624-626, 627-673, or a variant thereof, wherein: (a) the open reading frame is optimized for expression in an organism and the organism is different to the origin of the RT or endonuclease domain; or (b) the ORF comprises a sequence encoding an affinity tag. In some embodiments, the reverse transcriptase domain comprises a conserved xxDD, [F/Y]XDD, NAxxH, or VTG motif of any one of SEQ ID NOs: 609-610, 611-615, 616-617, 618-622, 623, 624-626, or 627-673. In some embodiments, the nucleic acid further encodes an RT having at least 80% sequence identity to any one of SEQ ID NOs: 612-613, 616-619, 622, 624, 627-630, 633, or a variant thereof. In some embodiments, the ORF comprises a sequence encoding an affinity tag. In some embodiments, the open reading frame comprises a sequence having at least 80% sequence identity to any one of SEQ ID NOs: 308-309, 310-312, 313-314, 315-319, 320, 321-323, or 174-180. In some embodiments, the organism is different to the origin of the RT domain. In some embodiments, the ORF comprises a sequence having at least 80% sequence identity to any one of SEQ ID NOs: 324-325, 326-328, 329-330, 331-335, 336, 327-329, or 181-187.

In some aspects, the present disclosure provides for a synthetic oligonucleotide comprising at least six consecutive nucleotides having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 340-341, 342-344, 345-346, 347-351, 352, or 353-355. In some embodiments, the synthetic oligonucleotide comprises DNA nucleotides. In some embodiments, the oligonucleotide further comprises at least one phosphorothioate linkage.

In some aspects, the present disclosure provides for a vector comprising a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 340-341, 342-344, 345-346, 347-351, 352, or 353-355.

In some aspects, the present disclosure provides for a vector comprising any of the nucleic acids described herein.

In some aspects, the present disclosure provides for a host cell comprising any of the nucleic acids described herein. In some embodiments, the host cell is an E. coli cell. In some embodiments, the E. coli cell is a μDE3 lysogen or the E. coli cell is a BL21(DE3) strain. In some embodiments, the E. coli cell has an ompT ion genotype. In some embodiments, the nucleic acid comprises an open reading from (ORF) encoding a retrotransposase, a fragment thereof, or a reverse transcriptase domain, wherein the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, the open reading frame comprises a sequence encoding an affinity tag linked in-frame to a sequence encoding the retrotransposase, the fragment thereof, or the reverse transcriptase domain.

In some aspects, the present disclosure provides for a culture comprising any of the host cells described herein in compatible liquid medium.

In some aspects, the present disclosure provides for a method of producing a retrotransposase, a fragment thereof, or a reverse transcriptase domain comprising cultivating any of the host cells described herein in compatible liquid medium. In some embodiments, the method further comprises inducing expression of the retrotransposase, the fragment thereof, or the reverse transcriptase domain by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to affinity chromatography specific to an affinity tag or ion-affinity chromatography.

In some aspects, the present disclosure provides for an in vitro transcribed mRNA comprising an RNA cognate of any the nucleic acids described herein.

In some aspects, the present disclosure provides for an engineered retrotransposase system, comprising: (a) a double-stranded nucleic acid comprising a cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase is derived from an uncultivated microorganism. In some embodiments, the cargo nucleotide sequence is engineered. In some embodiments, the cargo nucleotide sequence is heterologous. In some embodiments, the cargo nucleotide sequence does not have the sequence of a wild-type genome sequence present in an organism. In some embodiments, the retrotransposase comprises a sequence having at least 75% sequence identity to any one of SEQ ID NOs: 1-29. In some embodiments, the retrotransposase comprises a reverse transcriptase domain. In some embodiments, the retrotransposase further comprises one or more zinc finger domains. In some embodiments, the retrotransposase further comprises an endonuclease domain. In some embodiments, the retrotransposase has less than 80% sequence identity to a documented retrotransposase. In some embodiments, the cargo nucleotide sequence is flanked by a 3′ untranslated region (UTR) and a 5′ untranslated region (UTR). In some embodiments, the retrotransposase is configured to transpose the cargo nucleotide sequence via a ribonucleic acid polynucleotide intermediate. In some embodiments, the retrotransposase comprises one or more nuclear localization sequences (NLSs) proximal to an N- or C-terminus of the retrotransposase. In some embodiments, the NLS comprises a sequence at least 80% identical to a sequence selected from the group consisting of SEQ ID NO: 896-911. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for an engineered retrotransposase system, comprising: (a) a double-stranded nucleic acid comprising a cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; and (ii) the retrotransposase comprises a sequence having at least 75% sequence identity to any one of SEQ ID NOs: 1-29 In some embodiments, the retrotransposase is derived from an uncultivated microorganism. In some embodiments, the retrotransposase comprises a reverse transcriptase domain. In some embodiments, the retrotransposase further comprises one or more zinc finger domains. In some embodiments, the retrotransposase further comprises an endonuclease domain. In some embodiments, the retrotransposase has less than 80% sequence identity to a documented retrotransposase. In some embodiments, the cargo nucleotide sequence is flanked by a 3′ untranslated region (UTR) and a 5′ untranslated region (UTR). In some embodiments, the retrotransposase is configured to transpose the cargo nucleotide sequence via a ribonucleic acid polynucleotide intermediate. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for a deoxyribonucleic acid polynucleotide encoding the engineered retrotransposase system of any one of the aspects or embodiments described herein.

In some aspects, the present disclosure provides for a nucleic acid comprising an engineered nucleic acid sequence optimized for expression in an organism, wherein the nucleic acid encodes a retrotransposase, and wherein the retrotransposase is derived from an uncultivated microorganism, wherein the organism is not the uncultivated microorganism. In some embodiments, the retrotransposase comprises a variant having at least 75% sequence identity to any one of SEQ ID NOs: 1-29. In some embodiments, the retrotransposase comprises a sequence encoding one or more nuclear localization sequences (NLSs) proximal to an N- or C-terminus of the retrotransposase. In some embodiments, the NLS comprises a sequence selected from SEQ ID NOs: 896-911. In some embodiments, the NLS comprises SEQ ID NO: 897. In some embodiments, the NLS is proximal to the N-terminus of the retrotransposase. In some embodiments, the NLS comprises SEQ ID NO: 896. In some embodiments, the NLS is proximal to the C-terminus of the retrotransposase. In some embodiments, the organism is prokaryotic, bacterial, eukaryotic, fungal, plant, mammalian, rodent, or human

In some aspects, the present disclosure provides for a vector comprising the nucleic acid of any one of the aspects or embodiments described herein. In some embodiments, the vector further comprises a nucleic acid encoding a cargo nucleotide sequence configured to form a complex with the retrotransposase. In some embodiments, the vector is a plasmid, a minicircle, a CELiD, an adeno-associated virus (AAV) derived virion, or a lentivirus.

In some aspects, the present disclosure provides for a cell comprising the vector of any one of any one of the aspects or embodiments described herein.

In some aspects, the present disclosure provides for a method of manufacturing a retrotransposase, comprising cultivating the cell of any of the aspects or embodiments described herein.

In some aspects, the present disclosure provides for a method for binding, nicking, cleaving, marking, modifying, or transposing a double-stranded deoxyribonucleic acid polynucleotide, comprising: (a) contacting the double-stranded deoxyribonucleic acid polynucleotide with a retrotransposase configured to transpose the cargo nucleotide sequence to a target nucleic acid locus; wherein the retrotransposase comprises a sequence having at least 75% sequence identity to any one of SEQ ID NOs: 1-29. In some embodiments, the retrotransposase is derived from an uncultivated microorganism. In some embodiments, the retrotransposase comprises a reverse transcriptase domain. In some embodiments, the retrotransposase further comprises one or more zinc finger domains. In some embodiments, the retrotransposase further comprises an endonuclease domain. In some embodiments, the retrotransposase has less than 80% sequence identity to a documented retrotransposase. In some embodiments, the cargo nucleotide sequence is flanked by a 3′ untranslated region (UTR) and a 5′ untranslated region (UTR). In some embodiments, the double-stranded deoxyribonucleic acid polynucleotide is transposed via a ribonucleic acid polynucleotide intermediate. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides for a method of modifying a target nucleic acid locus, the method comprising delivering to the target nucleic acid locus the engineered retrotransposase system of any one of the aspects or embodiments described herein, wherein the retrotransposase is configured to transpose the cargo nucleotide sequence to the target nucleic acid locus, and wherein the complex is configured such that upon binding of the complex to the target nucleic acid locus, the complex modifies the target nucleic acid locus In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA). In some embodiments, the target nucleic acid locus comprises genomic DNA, viral DNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC).

In some aspects, the present disclosure provides for a method of any one of the aspects or embodiments described herein, wherein delivering the engineered retrotransposase system to the target nucleic acid locus comprises delivering the nucleic acid of any one of the aspects or embodiments described herein or the vector of any of the aspects or embodiments described herein. In some embodiments, delivering the engineered retrotransposase system to the target nucleic acid locus comprises delivering a nucleic acid comprising an open reading frame encoding the retrotransposase. In some embodiments, the nucleic acid comprises a promoter to which the open reading frame encoding the retrotransposase is operably linked. In some embodiments, delivering the engineered retrotransposase system to the target nucleic acid locus comprises delivering a capped mRNA containing the open reading frame encoding the retrotransposase. In some embodiments, delivering the engineered retrotransposase system to the target nucleic acid locus comprises delivering a translated polypeptide. In some embodiments, the retrotransposase does not induce a break at or proximal to the target nucleic acid locus.

In some aspects, the present disclosure provides for a host cell comprising an open reading frame encoding a heterologous retrotransposase having at least 75% sequence identity to any one of SEQ ID NOs: 1-29 or a variant thereof. In some embodiments, the host cell is an E. coli cell. In some embodiments, the E. coli cell is a λDE3 lysogen or the E. coli cell is a BL21(DE3) strain. In some embodiments, the E. coli cell has an ompT lon genotype. In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, the open reading frame comprises a sequence encoding an affinity tag linked in-frame to a sequence encoding the retrotransposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the retrotransposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell

In some aspects, the present disclosure provides for a culture comprising the host cell of any one of the aspects or embodiments described herein in compatible liquid medium.

In some aspects, the present disclosure provides for a method of producing a retrotransposase, comprising cultivating the host cell of any one of the aspects or embodiments described herein in compatible growth medium. In some embodiments, the method further comprises inducing expression of the retrotransposase by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprising isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the retrotransposase. In some embodiments, the IMAC affinity tag is linked in-frame to the sequence encoding the retrotransposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the IMAC affinity tag by contacting a protease corresponding to the protease cleavage site to the retrotransposase. In some embodiments, the method further comprises performing subtractive IMAC affinity chromatography to remove the affinity tag from a composition comprising the retrotransposase.

In some aspects, the present disclosure provides for a method of disrupting a locus in a cell, comprising contacting to the cell a composition comprising: (a) a double-stranded nucleic acid comprising a cargo nucleotide sequence, wherein the cargo nucleotide sequence is configured to interact with a retrotransposase; and (b) a retrotransposase, wherein: (i) the retrotransposase is configured to transpose the cargo nucleotide sequence to a target nucleic acid locus; (ii) the retrotransposase comprises a sequence having at least 75% sequence identity to any one of SEQ ID NOs: 1-29; and (iii) the retrotransposase has at least equivalent transposition activity to a documented retrotransposase in a cell. In some embodiments, the transposition activity is measured in vitro by introducing the retrotransposase to cells comprising the target nucleic acid locus and detecting transposition of the target nucleic acid locus in the cells. In some embodiments, the composition comprises 20 pmoles or less of the retrotransposase. In some embodiments, the composition comprises 1 pmol or less of the retrotransposase.

In some aspects, the present disclosure provides for a host cell comprising an open reading frame encoding any of the proteins described herein. In some embodiments, the host cell is an E. coli cell or a mammalian cell. In some embodiments, the host cell is an E. coli cell, wherein the E. coli cell is a λDE3 lysogen or the E. coli cell is a BL21(DE3) strain. In some embodiments, the E. coli cell has an ompT ion genotype. In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, the open reading frame comprises a sequence encoding an affinity tag linked in-frame to a sequence encoding the protein. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a strep tag, a FLAG tag, or any combination thereof. In some embodiments, the affinity tag is linked in-frame to the sequence encoding the protein via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell.

In some aspects, the present disclosure provides for a culture comprising any of the host cells described herein in compatible liquid medium.

In some aspects, the present disclosure provides for a method of producing any of the proteins described herein, comprising cultivating any of the host cells described herein encoding any of the proteins described herein in compatible growth medium. In some embodiments, the method further comprises inducing expression of the protein. In some embodiments, the inducing expression of the nuclease is by addition of an additional chemical agent or an increased amount of a nutrient, or by temperature increase or decrease. In some embodiments, an additional chemical agent or an increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract comprising the protein. In some embodiments, the method further comprises isolating the protein. In some embodiments, the isolating comprises subjecting the protein extract to IMAC, ion-exchange chromatography, anion exchange chromatography, or cation exchange chromatography. In some embodiments, the host cell comprises a nucleic acid comprising an open reading frame comprising a sequence encoding an affinity tag linked in-frame to a sequence encoding the protein. In some embodiments, the affinity tag is linked in-frame to the sequence encoding the protein via a linker sequence encoding a protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the affinity tag by contacting a protease corresponding to the protease cleavage site to the protein. In some embodiments, the affinity tag is an IMAC affinity tag. In some embodiments, the method further comprises performing subtractive IMAC affinity chromatography to remove the affinity tag from a composition comprising the protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the genomic context of a bacterial retrotransposon. MG140-1 is a predicted retrotransposase (arrow) encoding a Zn-finger DNA binding domain and a reverse transcriptase domain. Regions flanking the retrotransposase display secondary structure that possibly represent binding sites for the retrotransposase (Secondary structure boxes and zoomed images). Regions of similarity with other homologs indicate putative target sites at which the retrotransposon integrated.

FIG. 2A and FIG. 2B depict multiple sequence alignment (MSA) of MG retrotransposase protein sequences of the family MG140. FIG. 2A depicts MSA of the reverse transcriptase domain. Conserved catalytic residues D, QG, [Y/F]ADD, and LG are highlighted on the consensus sequence. FIG. 2B depicts MSA of a Zn-finger and endonuclease domains. Zn-finger motifs (CX_{[2-3]}C), part of the endonuclease domain and nuclease catalytic residues are highlighted on the consensus sequence.

FIG. 3A and FIG. 3B depict a phylogenetic gene tree of MG and reference retrotransposase genes. FIG. 3A depicts microbial MG retrotransposases (black branches on clade 4) are more closely related to Eukaryotic than viral retrotransposases (grey branches on clade 6). Clade 1: Telomerase reverse transcriptases; clade 2: Group II intron reverse transcriptases; clade 3: Eukaryotic R1 type retrotransposases; clade 4: microbial and Eukaryotic R2 retrotransposases; clade 5: Eukaryotic retrovirus-related reverse transcriptases; and clade 6: viral reverse transcriptases. FIG. 3B depicts Clades 3 and 4 from the phylogenetic gene tree from FIG. 3A. Some microbial MG retrotransposases contain multiple Zn-finger motifs (vertical rectangles), the conserved RVT_1 reverse transcriptase domain, and APE/RLE or other endonuclease domains (top and bottom panel). Some microbial MG retrotransposases lack an endonuclease domain (mid-panel).

FIG. 4 depicts a phylogenetic tree inferred from a multiple sequence alignment of the reverse transcriptase domain from diverse enzymes. RT sequences were derived from DNA, as well as RNA assemblies. Reference RTs were included in the tree for classification purposes.

FIG. 5A depicts a phylogenetic tree inferred from a multiple sequence alignment of RT domains identified from novel families of non-LTR retrotransposases (MG140, MG146 and MG147) and related RTs (MG148). FIG. 5B depicts data demonstrating that non-LTR retrotransposases (MG140, MG146 and MG147) contain an RT domain, an endonuclease domain (Endo), and multiple zinc-binding ribbon motifs, while family MG148 RTs lack an endonuclease domain.

FIG. 6A depicts data demonstrating that MG140 R2 retrotransposases contain RT and endonuclease (EN) domains, as well as multiple zinc-fingers, and share between 24% and 26% average amino acid identity (AAI) with the reference Danio rerio R2 retrotransposase (R2Dr).

FIG. 6B depicts data demonstrating that the MG140-47 R2 retrotransposon integrates into 28S rRNA gene. Alignment of the MG140-47 contig to a reference (GQ398061) ribosomal RNA operon shows a large gap in the reference 28S rDNA gene due to integration of the R2 element into the MG140-47 28S rDNA gene (dotted box).

FIG. 7A depicts genomic context of the MG145-45 retrotransposon. The enzyme contains RT and Zinc-finger domains. A partial 18S rDNA gene hit at the 5′ end and poly-A tail at the 3′ end likely delineate the boundaries of the transposon. FIG. 7B depicts alignment of MG140-3, MG140-8, and MG140-45 genomic sequences, showing conservation of the 18S rRNA gene to position 200 of the alignment and indicating integration of the R2 elements into the 18S rDNA gene (arrow).

FIG. 8A depicts the contig encoding the MG146-1 retrotransposase with RT and endonuclease domains. FIG. 8B depicts the MG140-17-R2 retrotransposon encoding three genes predicted to be involved in mobilization: RNA recognition motif gene (RRM); endonuclease enzyme; and reverse transcriptase with RT and RNAse H domains.

FIG. 9A depicts genomic context of two members of the MG148 family of RTs. Predicted genes not associated with the RT are displayed as white arrows. FIG. 9B depicts nucleotide sequence alignment of five members of the MG148 family indicating conserved regions (boxes underneath the sequence) upstream of the RT (arrow annotated over the consensus sequence).

FIG. 10 depicts screening of in vitro activity of RTns family of enzymes by qPCR (MG140). Activity was detected by qPCR using primers that amplify the full-length cDNA product derived from a primer extension reaction containing the respective RT. Samples are derived from RT reactions containing 100 nM substrate. Negative control: no-template water control in the PURExpress reaction; positive control 1: R2Tg (Taeniopygia guttata); positive control 2: R2Bm (Bombyx mori). The two positive controls are documented R2 retrotransposons. Active candidates, defined as at least 10-fold signal above the negative control, are marked in dark grey while candidates inactive in these conditions are in light grey.

FIG. 11 depicts screening of in vitro activity of RTns family of enzymes by qPCR (MG146, MG147, MG148). Activity was detected by qPCR using primers that amplify the full-length cDNA product derived from a primer extension reaction containing the respective RT. Samples are derived from RT reactions containing 100 nM substrate. Negative control: no-template water control in the PURExpress reaction; positive control 1: R2Tg (Taeniopygia guttata), a documented R2 retrotransposon. Active candidates, defined as at least 10-fold signal above the negative control, are marked in dark grey while candidates inactive in these conditions are in light grey.

FIG. 12 depicts an assay to assess the fidelity of R2 and R2-like candidates by next generation sequencing. The resulting cDNA product from a primer extension reaction was PCR-amplified and library prepped for NGS. Trimmed reads were aligned to the reference sequence and the frequency of misincorporation was calculated. Background: no-template water control in the PURExpress reaction; positive control 1: R2Tg (Taeniopygia guttata).

FIG. 13A depicts a phylogenetic tree inferred from a multiple sequence alignment of full-length Group II intron RTs identified from novel families from diverse classes. FIG. 13B depicts a summary table of MG families of Group II introns. AAI: average pairwise amino acid identity of MG families to reference Group II intron sequences.

FIGS. 14A-14D depict screening of in vitro activity of GII intron Class C candidates MG153-1 through MG153-21 and MG153-25 through MG153-27 by primer extension assay. For FIG. 14A through FIG. 14C, lane numbers correspond to the following: 1-PURExpress no template control, 2-MMLV control RT, 3-TGIRT-III control RT, 4-MarathonRT control RT. Numbering in bold corresponds to gel lanes with active novel candidates. Results are representative of two independent experiments. FIG. 14A lane numbers 5-14 correspond to novel candidates MG153-1 through MG153-10. FIG. 14B lane numbers 5-14 correspond to novel candidates MG153-11 through MG153-20. FIG. 14C lane numbers 5-8 correspond to novel candidates MG153-21, MG153-25, MG153-26, and MG153-27, respectively. FIG. 14D depicts detection of full-length cDNA production by qPCR. Dark grey bars correspond to RTs that generate product at least 10-fold above background. Results were determined from two technical replicates. Arrows in FIG. 14A through FIG. 14C indicate full-length cDNA product (arrow near the top of the gel) and examples of cDNA drop off (lower arrows).

FIGS. 15A-15D depict screening of in vitro activity of GII intron Class C candidates MG153-28 through MG153-37 and MG153-39 through MG153-57 by primer extension assay. For FIG. 15A through FIG. 15C, lane numbers correspond to the following: 1-PURExpress no template control, 2-MMLV control RT, 3-TGIRT-III control RT. Numbering in bold corresponds to gel lanes. FIG. 15A lane numbers 4-13 correspond to novel candidates MG153-28 through MG153-37. FIG. 15B lane numbers 4-13 correspond to novel candidates MG153-39 through MG153-48. FIG. 15C lane numbers 4-13 correspond to novel candidates MG153-49 through MG153-57. FIG. 15D depicts detection of full-length cDNA production by qPCR. Dark grey bars correspond to RTs that generate product at least 10-fold above background. Results were determined from two technical replicates. Arrows in FIG. 15A through FIG. 15C indicate full-length cDNA product (arrow near the top of the gel) and examples of cDNA drop off (lower arrows).

FIG. 16A and FIG. 16B depict screening of in vitro activity of GII intron Class D MG165 family of reverse transcriptases by primer extension assay. For FIG. 16A, lane numbers correspond to the following: 1-PURExpress no template control, 2-MMLV control RT, 3-TGIRT-III control RT, 4 through 12-novel candidates MG165-1 through 9. Numbering in bold corresponds to gel lanes with active novel candidates. FIG. 16B depicts quantification of full-length cDNA production by qPCR. Dark grey bars correspond to RTs that generate product at least 10-fold above background. Results were determined from two technical replicates. Arrows in FIG. 16A indicate full-length cDNA product (arrow near the top of the gel) and examples of cDNA drop off (lower arrows).

FIG. 17A and FIG. 17B depict screening of in vitro activity of GII intron Class F MG167 family of reverse transcriptases by primer extension assay. For FIG. 17A, lane numbers correspond to the following: 1-PURExpress no template control, 2-MMLV control RT, 3-TGIRT-III control RT, 4 through-novel candidates MG167-1 through 8. Numbering in bold corresponds to gel lanes with active novel candidates. FIG. 17B depicts quantification of full-length cDNA production by qPCR. Dark grey bars correspond to RTs that generate product at least 10-fold above background. Results were determined from two technical replicates. Arrows in FIG. 17A indicate full-length cDNA product (arrow near the top of the gel) and examples of cDNA drop off (lower arrows).

FIG. 18 depicts an assay to assess the fidelity of GII intron Class C RT candidates from the MG153 family by next generation sequencing. The resulting cDNA product from a primer extension reaction was PCR-amplified and library prepped for NGS. Trimmed reads were aligned to the reference sequence and the frequency of misincorporation was calculated. Results were determined from two independent experiments.

FIGS. 19A-19C depict screening to assess the ability of indicated control RTs and GII intron Class C candidates to synthesize cDNA in mammalian cells. FIG. 19A depicts detection of 542 bp (top) and 100 bp (bottom) PCR products by agarose gel analysis. FIG. 19B depicts detection of 542 bp (top) and 100 bp (bottom) PCR products by D1000 TapeStation. FIG. 19C depicts detection of 542 bp PCR products by D1000 TapeStation for additional candidates. Lanes not relevant for the described experiment in FIG. 19A and FIG. 19B are covered by black boxes.

FIG. 20A depicts a phylogenetic tree of full-length G2L4-like RTs. Reference G2L4 sequences and MG172 candidates (dots) are highlighted. FIG. 20B depicts data demonstrating that columns 277 to 280 of reference and MG172 RTs represent the catalytic residues responsible for reverse transcriptase function.

FIG. 21A depicts a phylogenetic tree of full-length LTR RTs. Reference LTR RT sequences and MG151 candidates (dots) are highlighted. FIG. 21B depicts genomic context of MG151-82 RT (labeled ORF 7). Predicted domains are shown as dark boxes and long terminal repeats (LTR) are shown as arrows flanking the LTR transposon. FIG. 21C depicts 3D structure prediction of MG151-82 showing the protease, RT, RNAse H and integrase domains.

FIG. 22 depicts multiple sequence alignment of full-length pol protein sequences to highlight the protease, RT-RNAse H, and integrase domains. Catalytic residues for the RT, RNAse H, and integrase domains of the MMLV RT are shown by bars under each domain. The protease domain of the MMLV reference sequence is not shown in the alignment.

FIGS. 23A-23C depict screening of in vitro activity of viral candidates MG151-80 through MG151-97 by primer extension assay. For FIG. 23A, lane numbers correspond to the following: 1-RNA template annealed to primer; 2-MMLV control RT; 3-Ty3 control RT; 4 through 9 novel candidates MG151-80 through 85; 10-RT control. For FIG. 23B, lane numbers correspond to the following: 1-RNA template annealed to primer, 2 through 12-novel candidates MG151-87 through 97, 13-MMLV control RT. FIG. 23C depicts testing of in vitro activity of Ty3 control RT in different buffer conditions. Lane numbers correspond to the following: 1-PURExpress no template control; 2-Buffer A (40 mM Tris-HCl pH 7.5, 0.2 M NaCl, 10 mM MgCl₂, 1 mM TCEP); 3-Buffer B (20 mM Tris pH 7.5, 150 mM KCl, 5 mM MgCl₂, 1 mM TCEP, 2% PEG-8000); 4-Buffer C (10 mm Tris-HCl pH 7.5, 80 mm NaCl, 9 mm MgCl₂, 1 mM TCEP, 0.01% (v/v) Triton X-100); 5-Buffer D (10 mM Tris pH 7.5, 130 mM NaCl, 9 mM MgCl₂, 1 mM TCEP, 10% glycerol). Arrows in FIG. 23A through FIG. 23C indicate full-length cDNA product (arrow near the top of the gel) and examples of cDNA drop off (lower arrows).

FIG. 24A and FIG. 24B depict testing of in vitro RT processivity and priming parameters of candidates MG151-89, MG151-92, and MG151-97 on a structured RNA template. For FIG. 24A and FIG. 24B, lane 1: 6, 10, and 16 nucleotide oligo markers (arrows); lane 2: 8, 13, and 20 nucleotide oligo marker; lane 3: 43 and 55 nucleotide oligo marker; lanes 4 and 10: 6 nucleotide primer; lanes 5 and 11: 8 nucleotide primer; lanes 6 and 12: 10 nucleotide primer; lanes 7 and 13: 13 nucleotide primer; lanes 8 and 14: 16 nucleotide primer; lanes 9 and 15: 20 nucleotide primer. FIG. 24A lanes 4-9 correspond to reverse transcription reactions containing MMLV with varying primer lengths. MMLV reverse transcribes through the structured RNA hairpin. Lanes 10-15 correspond to reverse transcription reactions containing MG151-89 with varying primer lengths. MG151-89 prefers primer lengths of 16 and 20 nucleotides and appears to stop reverse transcription at the structured RNA hairpin. FIG. 24B lanes 4-9 correspond to reverse transcription reactions containing MG151-92 with varying primer lengths. Lanes 10-15 correspond to reverse transcription reactions containing MG151-97 with varying primer lengths. Neither MG151-92 or MG151-97 appear active under these experimental conditions.

FIG. 25 depicts phylogenetic analysis of 2407 Retron RTs, with the first candidates selected for downstream characterization in vitro highlighted. 9 of 16 experimentally validated retrons in the literature were added and highlighted in the tree. Grey stars represent candidate MG154-MG159 and MG173 family members.

FIG. 26 depicts protein alignment of some Retron-RTs candidates selected for downstream characterization in vitro. Retron-specific motifs and the catalytic XXDD core common to all documented reverse transcriptases are indicated on the figure.

FIG. 27A depicts genomic context of the MG157-1 retron (arrow labeled RT on a thick black line). Retron non-coding RNA (ncRNA) is highlighted with a dotted box. FIG. 27B depicts an inset showing the MG157-1 retron ncRNA with it's flanking inverted repeats. FIG. 27C depicts the predicted structure of the MG157-1 retron ncRNA.

FIG. 28A depicts genomic context of the MG160-3 retron-like single-domain RT. The region upstream from the RT (dotted box) is conserved across MG160 members. FIG. 28B depicts 3D structure prediction of MG160-3 showing the RT domain aligned to a group II intron cryo-EM structure. FIG. 28C depicts predicted structures of the 5′ UTR of five MG160 members.

FIG. 29A and FIG. 29B depict screening of in vitro activity of retron-like candidates MG160-1 through MG160-6 and MG160-8 by primer extension assay. FIG. 29A lane numbers correspond to the following samples: 1-PURExpress no template control, 2-MMLV control RT, 3-TGIRT-III control RT, 4 through 10-novel candidates MG160-1 through MG160-6 and MG160-8. Numbering in bold corresponds to gel lanes with active novel candidates. FIG. 29B depicts quantification of full-length cDNA production by qPCR. Dark grey bars correspond to RTs that generate product at least 10-fold above background. Results were determined from two technical replicates. Arrows in FIG. 29A indicate full-length cDNA product (arrow near the top of the gel) and examples of cDNA drop off (lower arrows).

FIGS. 30A-30C depict cell-free expression of retron RT candidates and generation of retron ncRNAs by in vitro transcription. FIG. 30A depicts confirmation of retron RT protein production in a cell-free expression system. Lanes correspond to the following: 1: ladder, 2: no template control, 3: MG156-1 (39 kDa), 4: MG156-2 (40 kDa), 5: MG157-1 (38 kDa). FIG. 30B depicts confirmation of retron RT protein production in a cell-free expression system. Lanes correspond to the following—1: ladder, 2: no template control, 3: MG157-2 (37 kDa), 4: MG157-5 (43 kDa), 5: MG159-1 (53 kDa), 6: Ec86 (38 kDa, positive control retron RT). FIG. 30C depicts generation of retron ncRNA templates by in vitro transcription. Lanes correspond to the following ncRNAs corresponding to the following retrons—1: MG154-1, 2: MG154-2, 3: MG155-1, 4: MG155-2, 5: MG155-3, 6: MG156-1, 7: MG156-2, 8: MG157-1, 9: MG157-2, 10: MG157-5, 11: MG158-1, 12: MG159-1, 13: Ec86, 14: MG155-4, 15: MG173-1, 16: MG155-5.

FIG. 31 depicts domain architecture demonstrating that the MG140-1 R2 retrotransposon integrates into 28S rRNA gene. The R2 retrotransposase (light grey arrow) contains multiple Zn-fingers, as well as RT and endonuclease domains. MG140-1 is flanked by 5′ and 3′ UTRs, which define the transposon boundaries. MG140-1 integrates precisely between the G and T nucleotides in the target site motif GGTAGC.

FIG. 32 depicts the testing of RT activity by primer extension with DNA oligo containing phosphorothioate bond modifications. Lane numbers correspond to the following, 1: PURExpress no template control with PS-modified Primer 1, 2: PURExpress no template control with PS-modified Primer 2, 3: PURExpress no template control with PS-modified Primer 3, 4: MMLV RT with unmodified primer, 5: MMLV RT with PS-modified primer 1, 6: MMLV RT with PS-modified primer 2, 7: MMLV RT with PS-modified primer 3, 8: TGIRT-III with unmodified primer, 9: TGIRT-III with PS-modified primer 1, 10: TGIRT-III with PS-modified primer 2, 11: TGIRT-III with PS-modified primer 3, 12: MG153-9 with unmodified primer, 13: MG153-9 with PS-modified primer 1, 14: MG153-9 with PS-modified primer 2, 15 MG153-9 with PS-modified primer 3. MMLV RT and TGIRT-III are control RTs.

FIG. 33 depicts the screening of activity of retron RTs on an RNA template by primer extension assay. Lane numbers correspond to the following, 1: PURExpress no template control, 2: MMLV control RT, 3: MG154-1, 4: MG155-1, 5: MG155-2, 6: MG155-3, 7: MG156-2, 8: MG157-1, 9: MG157-2, 10: MG157-5, 11: MG158-1, 12: MG159-1, 13: Ec86 control retron RT, 14: Sa163 control retron RT, 15: St85 control retron RT. Lanes in bold correspond to novel retron RTs that exhibit primer extension activity on the tested substrate.

FIG. 34 depicts the screening of the ability of MG153 GII derived RTs to synthesize cDNA in mammalian cells. Detection of 542 bp cDNA synthesis PCR products were assayed by Taqman qPCR. cDNA activity was normalized to the activity TGIRT control where TGIRT represents a value of 1. Y axis is shown in log 10 scale.

FIGS. 35A-35C depict protein expression of MG153 GII derived RTs by immunoblots. FIGS. 35A and 35B: Cells were transfected with plasmids containing the candidate RTs and protein expression was evaluated by immunoblot, detecting the HA peptide fused to the N termini of the RTs. All lanes were normalized to total protein concentration. White arrows point to bands at 2× the expected molecular size of the protein, which indicate protein dimers. Lanes not relevant for the described experiment in FIGS. 35A and 35B are covered by black boxes. FIG. 35C: Multiple sequence alignment of GII derived RT. The region shown corresponds to positions 196 through 201 of the alignment. The dimerization motif CAQQ is highlighted.

FIG. 36 depicts relative activity of GII derived RTs normalized to protein expression. cDNA synthesis was detected by Tagman qPCR, protein expression was detected by immunoblots. Activity relative to TGIRT was normalized per total protein concentration. Y axis is shown in a linear scale.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG140

SEQ ID NOs: 1-29 and 393-401 show the full-length peptide sequences of MG140 transposition proteins.

SEQ ID NOs: 374-386 show the nucleotide sequences of genes encoding HA-His-tagged MG140 reverse transcriptase proteins.

SEQ ID NOs: 761-798 show the nucleotide sequences of MG140 UTRs.

SEQ ID NOs: 799-894 show the full-length peptide sequences of MG140 reverse transcriptase proteins.

### MG146

SEQ ID NOs: 402 and 895 show the full-length peptide sequences of MG140 transposition proteins.

SEQ ID NO: 387 shows the nucleotide sequence of a gene encoding an HA-His-tagged MG146 reverse transcriptase protein.

### MG147

SEQ ID NO: 388 shows the nucleotide sequence of a gene encoding an HA-His-tagged MG147 reverse transcriptase protein.

### MG148

SEQ ID NOs: 403-426 show the full-length peptide sequences of MG148 reverse transcriptase proteins.

SEQ ID NOs: 389-392 show the nucleotide sequences of genes encoding HA-His-tagged MG148 reverse transcriptase proteins.

### MG149

SEQ ID NOs: 427-439 show the full-length peptide sequences of MG149 reverse transcriptase proteins.

### MG151

SEQ ID NOs: 440-554 show the full-length peptide sequences of MG151 reverse transcriptase proteins.

SEQ ID NOs: 356-362 show the nucleotide sequences of genes encoding TwinStrep-tagged MG151 reverse transcriptase proteins.

SEQ ID NOs: 363-373 show the nucleotide sequences of genes encoding strep-tagged MG151 reverse transcriptase proteins.

### MG153

SEQ ID NOs: 555-608 show the full-length peptide sequences of MG153 reverse transcriptase proteins.

SEQ ID NOs: 30-32 and 40-50 show the nucleotide sequences of fusion proteins comprising MG153 reverse transcriptase proteins and MS2 coat proteins (MCP).

SEQ ID NOs: 66-119 show the nucleotide sequences of genes encoding strep-tagged MG153 reverse transcriptase proteins.

SEQ ID NOs: 120-173 show the nucleotide sequences of E. coli codon optimized genes encoding MG153 reverse transcriptase proteins.

SEQ ID NOs: 740-756 show the nucleotide sequences of genes encoding MCP-tagged MG153 reverse transcriptase proteins.

### MG154

SEQ ID NOs: 609-610 show the full-length peptide sequences of MG154 reverse transcriptase proteins.

SEQ ID NOs: 308-309 show the nucleotide sequences of genes encoding strep-tagged MG154 reverse transcriptase proteins.

SEQ ID NOs: 324-325 show the nucleotide sequences of E. coli codon optimized genes encoding MG154 reverse transcriptase proteins.

SEQ ID NOs: 340-341 show the nucleotide sequences of ncRNAs compatible with MG154 nucleases.

### MG155

SEQ ID NOs: 611-615 show the full-length peptide sequences of MG155 reverse transcriptase proteins.

SEQ ID NOs: 310-312 show the nucleotide sequences of genes encoding strep-tagged MG155 reverse transcriptase proteins.

SEQ ID NOs: 326-328 show the nucleotide sequences of E. coli codon optimized genes encoding MG155 reverse transcriptase proteins.

SEQ ID NOs: 342-344 show the nucleotide sequences of ncRNAs compatible with MG155 nucleases.

### MG156

SEQ ID NOs: 616-617 show the full-length peptide sequences of MG156 reverse transcriptase proteins.

SEQ ID NOs: 313-314 show the nucleotide sequences of genes encoding strep-tagged MG156 reverse transcriptase proteins.

SEQ ID NOs: 329-330 show the nucleotide sequences of E. coli codon optimized genes encoding MG156 reverse transcriptase proteins.

SEQ ID NOs: 345-346 show the nucleotide sequences of ncRNAs compatible with MG156 nucleases.

### MG157

SEQ ID NOs: 618-622 show the full-length peptide sequences of MG157 reverse transcriptase proteins.

SEQ ID NOs: 315-319 show the nucleotide sequences of genes encoding strep-tagged MG157 reverse transcriptase proteins.

SEQ ID NOs: 331-335 show the nucleotide sequences of E. coli codon optimized genes encoding MG157 reverse transcriptase proteins.

SEQ ID NOs: 347-351 show the nucleotide sequences of ncRNAs compatible with MG157 nucleases.

### MG158

SEQ ID NO: 623 shows the full-length peptide sequence of an MG158 reverse transcriptase protein.

SEQ ID NO: 320 shows the nucleotide sequence of a gene encoding a strep-tagged MG158 reverse transcriptase protein.

SEQ ID NO: 336 shows the nucleotide sequence of an E. coli codon optimized gene encoding an MG158 reverse transcriptase protein.

SEQ ID NO: 352 shows the nucleotide sequence of an ncRNA compatible with MG158 nucleases.

### MG159

SEQ ID NOs: 624-626 show the full-length peptide sequences of MG159 reverse transcriptase proteins.

SEQ ID NOs: 321-323 show the nucleotide sequences of genes encoding strep-tagged MG159 reverse transcriptase proteins.

SEQ ID NOs: 337-339 show the nucleotide sequences of E. coli codon optimized genes encoding MG159 reverse transcriptase proteins.

SEQ ID NOs: 353-355 show the nucleotide sequences of ncRNAs compatible with MG159 nucleases.

### MG160

SEQ ID NOs: 627-673 show the full-length peptide sequences of MG160 reverse transcriptase proteins.

SEQ ID NOs: 174-180 show the nucleotide sequences of genes encoding strep-tagged MG160 reverse transcriptase proteins.

SEQ ID NOs: 181-187 show the nucleotide sequences of E. coli codon genes encoding optimized MG160 reverse transcriptase proteins.

### MG163

SEQ ID NOs: 674-678 show the full-length peptide sequences of MG163 reverse transcriptase proteins.

SEQ ID NOs: 188-192 show the nucleotide sequences of genes encoding strep-tagged MG163 reverse transcriptase proteins.

SEQ ID NOs: 193-197 show the nucleotide sequences of E. coli codon genes encoding optimized MG163 reverse transcriptase proteins.

### MG164

SEQ ID NOs: 679-683 show the full-length peptide sequences of MG164 reverse transcriptase proteins.

SEQ ID NOs: 198-202 show the nucleotide sequences of genes encoding strep-tagged MG164 reverse transcriptase proteins.

SEQ ID NOs: 203-207 show the nucleotide sequences of E. coli codon genes encoding optimized MG164 reverse transcriptase proteins.

### MG165

SEQ ID NOs: 684-692 show the full-length peptide sequences of MG165 reverse transcriptase proteins.

SEQ ID NOs: 208-216 show the nucleotide sequences of genes encoding strep-tagged MG165 reverse transcriptase proteins.

SEQ ID NOs: 217-225 show the nucleotide sequences of E. coli codon genes encoding optimized MG165 reverse transcriptase proteins.

SEQ ID NOs: 757-759 show the nucleotide sequences of genes encoding MCP-tagged MG165 reverse transcriptase proteins.

### MG166

SEQ ID NOs: 693-697 show the full-length peptide sequences of MG166 reverse transcriptase proteins.

SEQ ID NOs: 226-230 show the nucleotide sequences of genes encoding strep-tagged MG166 reverse transcriptase proteins.

SEQ ID NOs: 231-235 show the nucleotide sequences of E. coli codon genes encoding optimized MG166 reverse transcriptase proteins.

### MG167

SEQ ID NOs: 698-702 show the full-length peptide sequences of MG167 reverse transcriptase proteins.

SEQ ID NOs: 236-240 show the nucleotide sequences of genes encoding strep-tagged MG167 reverse transcriptase proteins.

SEQ ID NOs: 241-245 show the nucleotide sequences of E. coli codon genes encoding optimized MG167 reverse transcriptase proteins.

SEQ ID NOs: 759-760 show the nucleotide sequences of genes encoding MCP-tagged MG167 reverse transcriptase proteins.

### MG168

SEQ ID NOs: 703-707 show the full-length peptide sequences of MG168 reverse transcriptase proteins.

SEQ ID NOs: 246-250 show the nucleotide sequences of genes encoding strep-tagged MG168 reverse transcriptase proteins.

SEQ ID NOs: 251-255 show the nucleotide sequences of E. coli codon genes encoding optimized MG168 reverse transcriptase proteins.

### MG169

SEQ ID NOs: 708-718 show the full-length peptide sequences of MG169 reverse transcriptase proteins.

SEQ ID NOs: 256-266 show the nucleotide sequences of genes encoding strep-tagged MG169 reverse transcriptase proteins.

SEQ ID NOs: 267-277 show the nucleotide sequences of E. coli codon genes encoding optimized MG169 reverse transcriptase proteins.

### MG170

SEQ ID NOs: 719-728 show the full-length peptide sequences of MG170 reverse transcriptase proteins.

SEQ ID NOs: 278-287 show the nucleotide sequences of genes encoding strep-tagged MG170 reverse transcriptase proteins.

SEQ ID NOs: 288-297 show the nucleotide sequences of E. coli codon genes encoding optimized MG170 reverse transcriptase proteins.

### MG172

SEQ ID NOs: 729-733 show the full-length peptide sequences of MG172 reverse transcriptase proteins.

SEQ ID NOs: 298-302 show the nucleotide sequences of genes encoding strep-tagged MG172 reverse transcriptase proteins.

SEQ ID NOs: 303-307 show the nucleotide sequences of E. coli codon genes encoding optimized MG172 reverse transcriptase proteins.

### MG173

SEQ ID NOs: 734-735 show the full-length peptide sequences of MG173 reverse transcriptase proteins.

### Other Sequences

SEQ ID NOs: 736-738 show the nucleotide sequences of phosphorothioate-modified primers.

SEQ ID NO: 739 shows the nucleotide sequence of a Tagman probe for qPCR.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, e.g., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional, or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Il.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some embodiments, the polymer may be interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions, or deletions. A non-native sequence may exhibit or encode for an activity (e.g., enzymatic activity, methyltransferase activity, acetyltransferase activity, kinase activity, ubiquitinating activity, etc.) that may also be exhibited by the nucleic acid or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, generally refers to the regulatory DNA region which controls transcription or expression of a gene, and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. Eukaryotic basal promoters can contain a TATA-box or a CAAT box.

The term “expression”, as used herein, generally refers to the process by which a nucleic acid sequence or a polynucleotide is transcribed from a DNA template (such as into mRNA or other RNA transcript) or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some embodiments, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” can generally be used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “transposable element” refers to a DNA sequence that can move from one location in the genome to another (e.g., they can be “transposed”). Transposable elements can be generally divided into two classes. Class I transposable elements, or “retrotransposons”, are transposed via transcription and translation of an RNA intermediate which is subsequently reincorporated into its new location into the genome via reverse transcription (a process mediated by a reverse transcriptase). Class II transposable elements, or “DNA transposons”, are transposed via a complex of single- or double-stranded DNA flanked on either side by a transposase. Further features of this family of enzymes can be found, e.g. in Nature Education 2008, 1 (1), 204; and Genome Biology 2018, 19 (199), 1-12; each of which is incorporated herein by reference.

As used herein, the term “retrotransposons” refers to Class I transposable elements that function according to a two-part “copy and paste” mechanism involving an RNA intermediate. “Retrotransposase” refers to an enzyme responsible for transposition of a retrotransposon. In some embodiments, a retrotransposase comprises a reverse transcriptase domain. In some embodiments, a retrotransposase further comprises one or more zinc finger domains. In some embodiments, a retrotransposase further comprises an endonuclease domain.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

The term “open reading frame” or “ORF” generally refers to a nucleotide sequence that can encode a protein, or a portion of a protein. An open reading frame can begin with a start codon (represented as, e.g. AUG for an RNA molecule and ATG in a DNA molecule in the standard code) and can be read in codon-triplets until the frame ends with a STOP codon (represented as, e.g. UAA, UGA, or UAG for an RNA molecule and TAA, TGA, or TAG in a DNA molecule in the standard code).

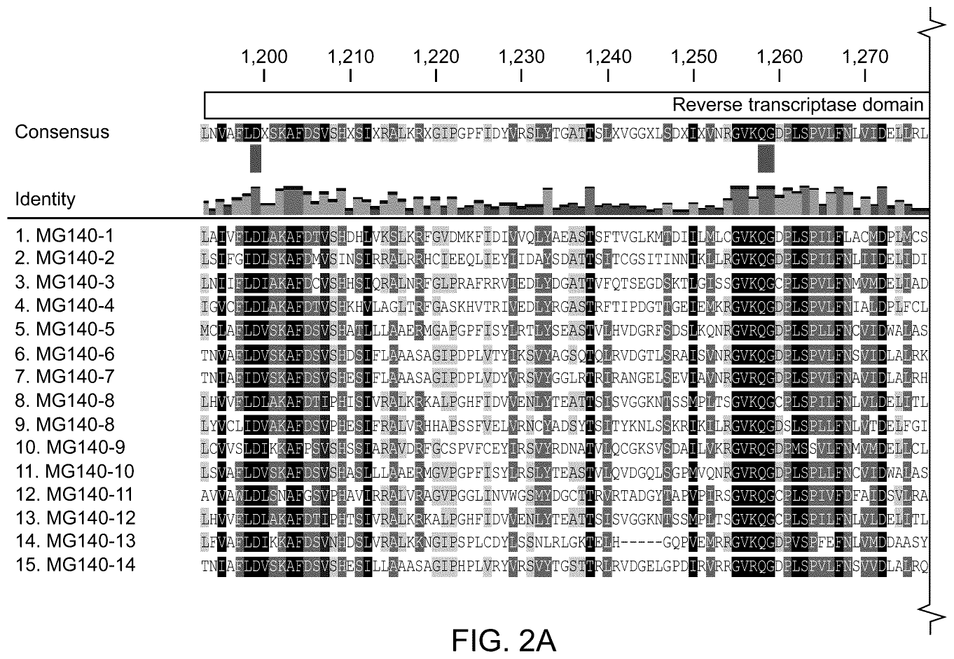

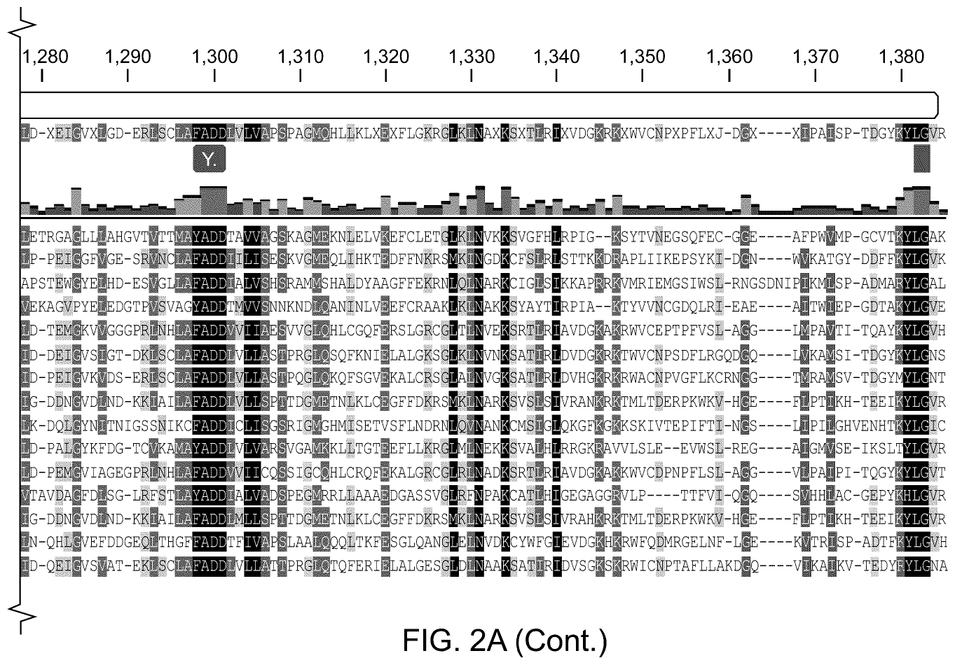

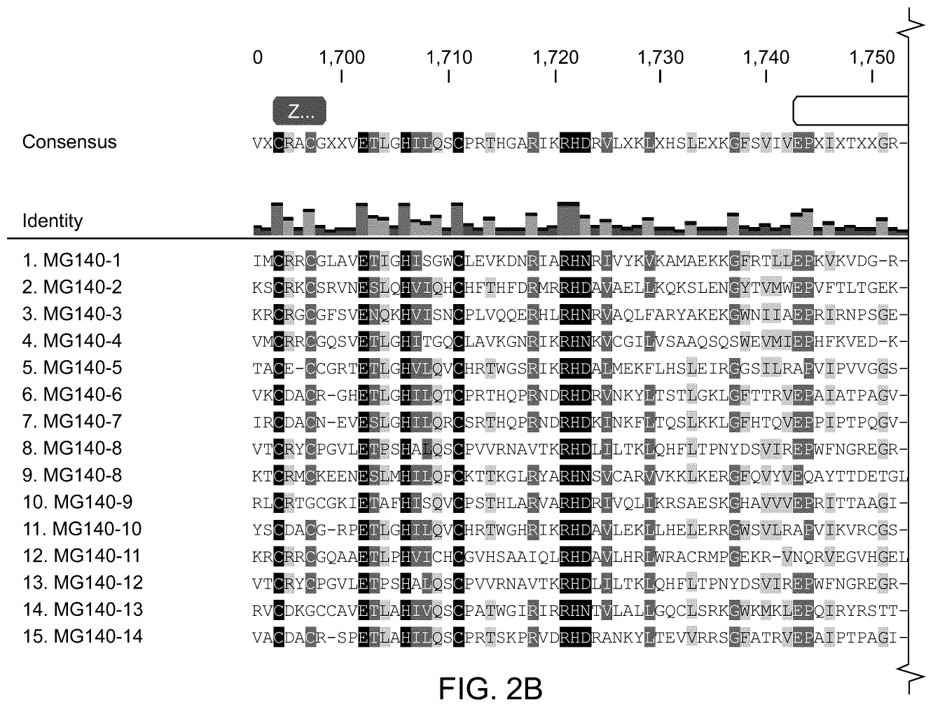

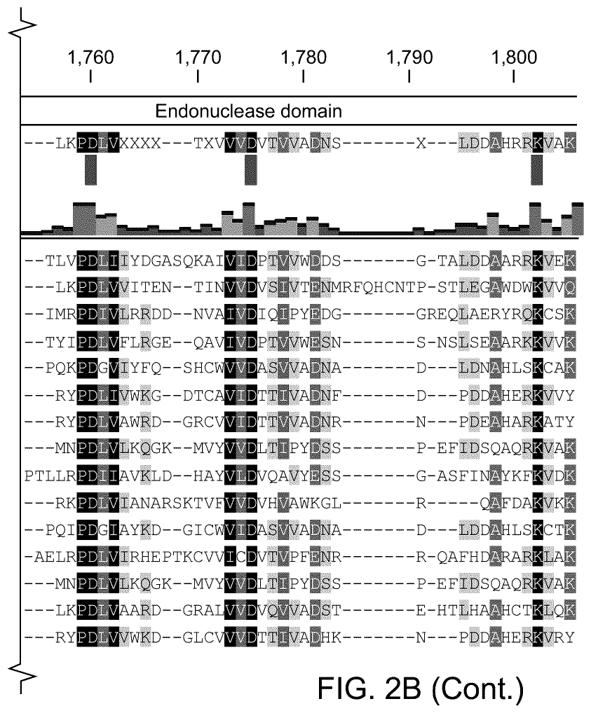

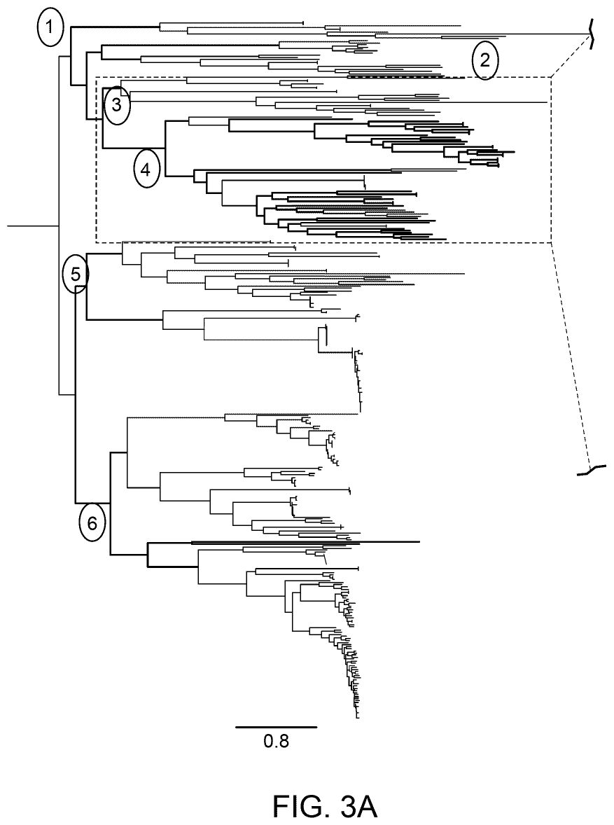

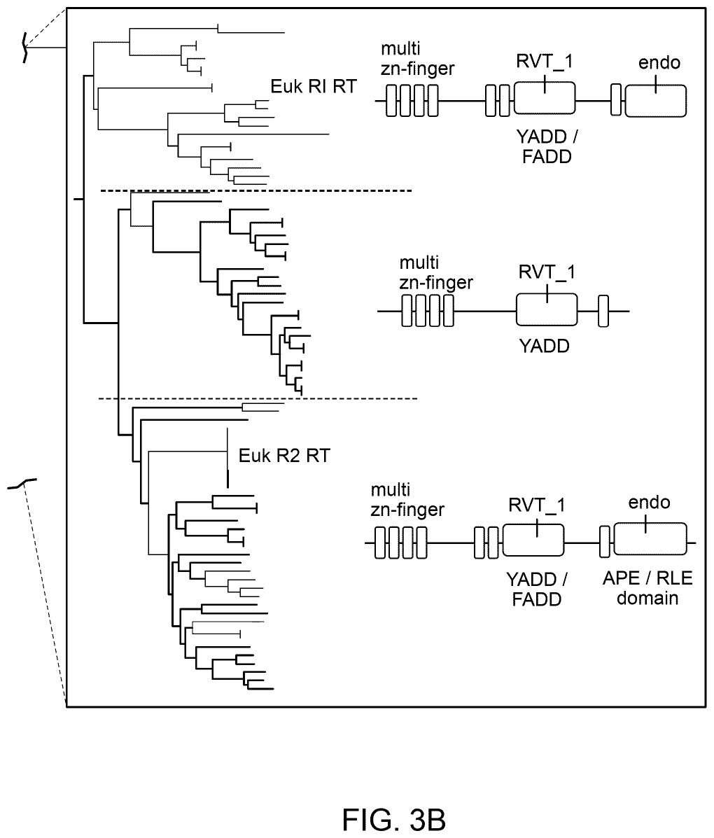

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of the retrotransposase protein sequences described herein (e.g. MG140 family retrotransposases described herein, or any other family retrotransposase described herein). In some embodiments, such conservatively substituted variants are functional variants. Such functional variants can encompass sequences with substitutions such that the activity of one or more critical active site residues of the retrotransposase are not disrupted. In some embodiments, a functional variant of any of the proteins described herein lacks substitution of at least one of the conserved or functional residues called out in FIG. 2A and FIG. 2B. In some embodiments, a functional variant of any of the proteins described herein lacks substitution of all of the conserved or functional residues called out in FIG. 2A and FIG. 2B.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues called out in FIG. 2A and FIG. 2B.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another: 1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

Also included in the current disclosure are variants of any of the nucleic acid sequences described herein with one or more substitutions, deletions, or insertions. In some embodiments, such a variant has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of the nucleic acid sequences described herein.

Some of the protein sequences described herein involve the determination of a particular domain (e.g. a reverse transcriptase or RT domain) from the sequence of a selected larger protein (e.g. a retrotransposase). In such cases, multiple sequence alignments (MSA) with a reference larger protein (e.g. a retrotransposase) where the domains have been validated (e.g. with 3D structures) is used to identify domain boundaries by aligning the selected protein to the larger protein with validated domains. When MSAs are inconclusive because the sequences are so divergent, 3D structures of the larger proteins are determined and the structural domains are compared with known domains to define the boundaries. These boundaries can be further verified by ensuring the presence of important catalytic residues for the domain within the domain boundaries.

As used herein, the term “LINE retrotransposase” generally refers to a class of autonomous non-LTR retrotransposons (Long INterspersed Element). As used herein, the term “R2 retrotransposase” or “R4 retrotransposase” generally refer to subclasses of LINE retrotransposases that share similar domain architecture but differ in that R2 retrotransposases can be site specific (e.g. integrating at specific sites of an rRNA gene) while R4 retrotransposons can integrate both at an rRNA gene as well as other non-specific sites containing repeats.

### Overview

The discovery of new transposable elements with unique functionality and structure may offer the potential to further disrupt deoxyribonucleic acid (DNA) editing technologies, improving speed, specificity, functionality, and ease of use. Relative to the predicted prevalence of transposable elements in microbes and the sheer diversity of microbial species, relatively few functionally characterized transposable elements exist in the literature. This is partly because a huge number of microbial species may not be readily cultivated in laboratory conditions.

Metagenomic sequencing from natural environmental niches containing large numbers of microbial species can offer the potential to drastically increase the number of new transposable elements documented and speed the discovery of new oligonucleotide editing functionalities.

Transposable elements are deoxyribonucleic acid sequences that can change position within a genome, often resulting in the generation or amelioration of mutations. In eukaryotes, a great proportion of the genome, and a large share of the mass of cellular DNA, is attributable to transposable elements. Although transposable elements are “selfish genes” which propagate themselves at the expense of other genes, they have been found to serve various important functions and to be crucial to genome evolution. Based on their mechanism, transposable elements are classified as either Class I “retrotransposons” or Class II “DNA transposons”.

Class I transposable elements, also referred to as retrotransposons, function according to a two-part “copy and paste” mechanism involving an RNA intermediate. First, the retrotransposon is transcribed. The resulting RNA is subsequently converted back to DNA by reverse transcriptase (generally encoded by the retrotransposon itself), and the reverse transcribed retrotransposon is integrated into its new position in the genome by integrase. Retrotransposons are further classified into three orders. Retrotransposons with long terminal repeats (“LTRs”) encode reverse transcriptase and are flanked by long strands of repeating DNA. Retrotransposons with long interspersed nuclear elements (“LINEs”) encode reverse transcriptase, lack LTRs, and are transcribed by RNA polymerase II. Retrotransposons with short interspersed nuclear elements (“SINEs”) are transcribed by RNA polymerase III but lack reverse transcriptase, instead relying on the reverse transcription machinery of other transposable elements (e.g. LINEs).

Class II transposable elements, also referred to as DNA transposons, function according to mechanisms that do not involve an RNA intermediate. Many DNA transposons display a “cut and paste” mechanism in which transposase binds terminal inverted repeats (“TIRs”) flanking the transposon, cleaves the transposon from the donor region, and inserts it into the target region of the genome. Others, referred to as “helitrons”, display a “rolling circle” mechanism involving a single-stranded DNA intermediate and mediated by an undocumented protein understood to possess HUH endonuclease function and 5′ to 3′ helicase activity. First, a circular strand of DNA is nicked to create two single DNA strands. The protein remains attached to the 5′ phosphate of the nicked strand, leaving the 3′ hydroxyl end of the complementary strand exposed and thus allowing a polymerase to replicate the non-nicked strand. Once replication is complete, the new strand disassociates and is itself replicated along with the original template strand. Still other DNA transposons, “Polintons”, are theorized to undergo a “self-synthesis” mechanism. The transposition is initiated by an integrase's excision of a single-stranded extra-chromosomal Polinton element, which forms a racket-like structure. The Polinton undergoes replication with DNA polymerase B, and the double stranded Polinton is inserted into the genome by the integrase. Additionally, some DNA transposons, such as those in the IS200/IS605 family, proceed via a “peel and paste” mechanism in which TnpA excises a piece of single-stranded DNA (as a circular “transposon joint”) from the lagging strand template of the donor gene and reinserts it into the replication fork of the target gene.

While transposable elements have found some use as biological tools, documented transposable elements do not encompass the full range of possible biodiversity and targetability, and may not represent all possible activities. Here, thousands of genomic fragments were mined from numerous metagenomes for transposable elements. The documented diversity of transposable elements may have been expanded and novel systems may have been developed into highly targetable, compact, and precise gene editing agents.

### MG Enzymes

In some aspects, the present disclosure provides for novel retrotransposases. These candidates may represent one or more novel subtypes and some sub-families may have been identified. These retrotransposases are less than about 1,400 amino acids in length. These retrotransposases may simplify delivery and may extend therapeutic applications.

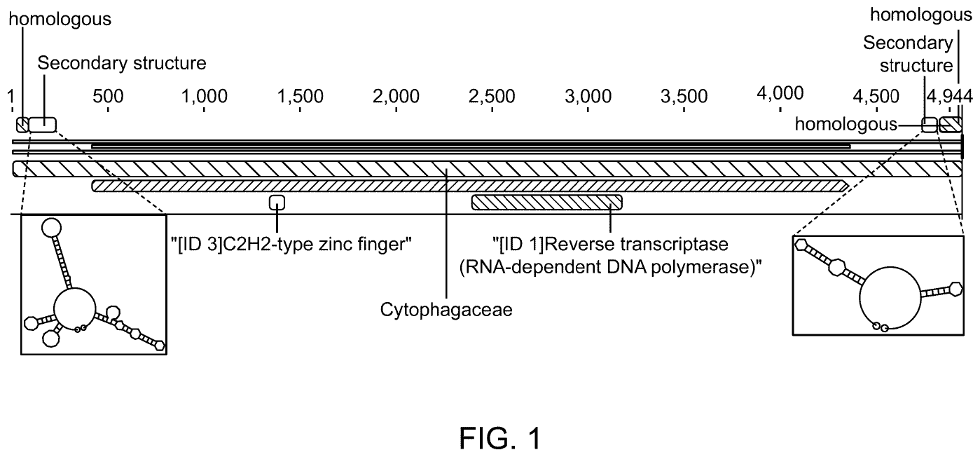

In some aspects, the present disclosure provides for a novel retrotransposase. Such a retrotransposase may be MG140 as described herein (see FIGS. 1 and 2).

In one aspect, the present disclosure provides for an engineered retrotransposase system discovered through metagenomic sequencing. In some embodiments, the metagenomic sequencing is conducted on samples. In some embodiments, the samples may be collected from a variety of environments. Such environments may be a human microbiome, an animal microbiome, environments with high temperatures, environments with low temperatures. Such environments may include sediment.

In one aspect, the present disclosure provides for an engineered retrotransposase system comprising a retrotransposase. In some embodiments, the retrotransposase is derived from an uncultivated microorganism. The retrotransposase may be configured to bind a 3′ untranslated region (UTR). The retrotransposase may bind a 5′ untranslated region (UTR).

In one aspect, the present disclosure provides for an engineered retrotransposase system comprising a retrotransposase. In some embodiments, the retrotransposase comprises a sequence having at least about 70% sequence identity to any one of SEQ ID NOs: 1-29, 393-735, or 799-895. In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895. In some embodiments, the retrotransposase may be substantially identical to any one of SEQ ID NOs: 1-29, 393-735, or 799-895.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a documented retrotransposase.

In some embodiments, the cargo nucleotide sequence is flanked by a 3′ untranslated region (UTR) and a 5′ untranslated region (UTR).

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase comprises a sequence complementary to a eukaryotic, fungal, plant, mammalian, or human genomic polynucleotide sequence. In some embodiments, the retrotransposase comprises a sequence complementary to a eukaryotic genomic polynucleotide sequence. In some embodiments, the retrotransposase comprises a sequence complementary to a fungal genomic polynucleotide sequence. In some embodiments, the retrotransposase comprises a sequence complementary to a plant genomic polynucleotide sequence. In some embodiments, the retrotransposase comprises a sequence complementary to a mammalian genomic polynucleotide sequence. In some embodiments, the retrotransposase comprises a sequence complementary to a human genomic polynucleotide sequence.

In some embodiments, the retrotransposase may comprise a variant having one or more nuclear localization sequences (NLSs). The NLS may be proximal to the N- or C-terminus of the retrotransposase. The NLS may be appended N-terminal or C-terminal to any one of SEQ ID NOs: 896-911, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 896-911. In some embodiments, the NLS may comprise a sequence substantially identical to any one of SEQ ID NOs: 896-911. In some embodiments, the NLS may comprise a sequence substantially identical to SEQ ID NO: 896. In some embodiments, the NLS may comprise a sequence substantially identical to SEQ ID NO: 897.

TABLE 1
Example NLS Sequences that may be used with retrotransposases according to the
disclosure

| Source | NLS amino acid sequence | SEQ ID NO: |
|---|---|---|
| SV40 | PKKKRKV | 896 |
| nucleoplasmin | KRPAATKKAGQAKKKK | 897 |
| bipartite NLS | | |
| c-myc NLS | PAAKRVKLD | 898 |
| c-myc NLS | RQRRNELKRSP | 899 |
| hRNPA1 M9 NLS | NQSSNFGPMKGGNFGGRSSGPYGGGGQYFAKPRNQGGY | 900 |
| Importin-alpha IBB | RMRIZFKNKGKDTAELRRRRVEVSVELRKAKKDEQILKRRNV | 901 |
| domain | | |
| Myoma T protein | VSRKRPRP | 902 |
| Myoma T protein | PPKKARED | 903 |
| p53 | PQPKKKPL | 904 |
| mouse c-abl IV | SALIKKKKKMAP | 905 |
| influenza virus NS1 | DRLRR | 906 |
| influenza virus NS1 | PKQKKRK | 907 |
| Hepatitis virus delta | RKLKKKIKKL | 908 |
| antigen | | |
| mouse Mx1 protein | REKKKELKRR | 909 |
| human poly (ADP- | KRKGDEVDGVDEVAKKKSKK | 910 |
| ribose) polymerase | | |
| steroid hormone | RKCLQAGMNLEARKTKK | 911 |
| receptors (human) | | |
| glucocorticoid | | |

In some embodiments, sequence may be determined by a BLASTP, CLUSTALW, MUSCLE, or MAFFT algorithm, or a CLUSTALW algorithm with the Smith-Waterman homology search algorithm parameters. The sequence identity may be determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In one aspect, the present disclosure provides a deoxyribonucleic acid polynucleotide encoding the engineered retrotransposase system described herein.

In one aspect, the present disclosure provides a nucleic acid comprising an engineered nucleic acid sequence. In some embodiments, the engineered nucleic acid sequence is optimized for expression in an organism. In some embodiments, the retrotransposase is derived from an uncultivated microorganism. In some embodiments, the organism is not the uncultivated organism.

In some embodiments, the retrotransposase comprises a sequence having at least about 70% sequence identity to any one of SEQ ID NOs: 1-29, 393-735, or 799-895. In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895. In some embodiments, the retrotransposase may be substantially identical to any one of SEQ ID NOs: 1-29, 393-735, or 799-895.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a documented retrotransposase.

In some embodiments, the cargo nucleotide sequence is flanked by a 3′ untranslated region (UTR) and a 5′ untranslated region (UTR).

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase comprises a sequence complementary to a eukaryotic, fungal, plant, mammalian, or human genomic polynucleotide sequence. In some embodiments, the retrotransposase comprises a sequence complementary to a eukaryotic genomic polynucleotide sequence. In some embodiments, the retrotransposase comprises a sequence complementary to a fungal genomic polynucleotide sequence. In some embodiments, the retrotransposase comprises a sequence complementary to a plant genomic polynucleotide sequence. In some embodiments, the retrotransposase comprises a sequence complementary to a mammalian genomic polynucleotide sequence. In some embodiments, the retrotransposase comprises a sequence complementary to a human genomic polynucleotide sequence.

In some embodiments, the retrotransposase may comprise a variant having one or more nuclear localization sequences (NLSs). The NLS may be proximal to the N- or C-terminus of the retrotransposase. The NLS may be appended N-terminal or C-terminal to any one of SEQ ID NOs: 896-911, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 896-911. In some embodiments, the NLS may comprise a sequence substantially identical to any one of SEQ ID NOs: 896-911. In some embodiments, the NLS may comprise a sequence substantially identical to SEQ ID NO: 896. In some embodiments, the NLS may comprise a sequence substantially identical to SEQ ID NO: 897.

In some embodiments, the organism is prokaryotic. In some embodiments, the organism is bacterial. In some embodiments, the organism is eukaryotic. In some embodiments, the organism is fungal. In some embodiments, the organism is a plant. In some embodiments, the organism is mammalian. In some embodiments, the organism is a rodent. In some embodiments, the organism is human.

In one aspect, the present disclosure provides an engineered vector. In some embodiments, the engineered vector comprises a nucleic acid sequence encoding a retrotransposase. In some embodiments, the retrotransposase is derived from an uncultivated microorganism.

In some embodiments, the engineered vector comprises a nucleic acid described herein. In some embodiments, the nucleic acid described herein is a deoxyribonucleic acid polynucleotide described herein. In some embodiments, the vector is a plasmid, a minicircle, a CELiD, an adeno-associated virus (AAV) derived virion, or a lentivirus.

In one aspect, the present disclosure provides a cell comprising a vector described herein.

In one aspect, the present disclosure provides a method of manufacturing a retrotransposase. In some embodiments, the method comprises cultivating the cell.

In one aspect, the present disclosure provides a method for binding, nicking, cleaving, marking, modifying, or transposing a double-stranded deoxyribonucleic acid polynucleotide. The method may comprise contacting the double-stranded deoxyribonucleic acid polynucleotide with a retrotransposase. In some embodiments, the cargo nucleotide sequence is flanked by a 3′ untranslated region (UTR) and a 5′ untranslated region (UTR).

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a documented retrotransposase.

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide.

In one aspect, the present disclosure provides a method of modifying a target nucleic acid locus. The method may comprise delivering to the target nucleic acid locus the engineered retrotransposase system described herein. In some embodiments, the complex is configured such that upon binding of the complex to the target nucleic acid locus, the complex modifies the target nucleic acid locus.

In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is a primary cell. In some embodiments, the primary cell is a T cell. In some embodiments, the primary cell is a hematopoietic stem cell (HSC).

In some embodiments, delivery of the engineered retrotransposase system to the target nucleic acid locus comprises delivering the nucleic acid described herein or the vector described herein. In some embodiments, delivery of engineered retrotransposase system to the target nucleic acid locus comprises delivering a nucleic acid comprising an open reading frame encoding the retrotransposase. In some embodiments, the nucleic acid comprises a promoter. In some embodiments, the open reading frame encoding the retrotransposase is operably linked to the promoter.

In some embodiments, delivery of the engineered retrotransposase system to the target nucleic acid locus comprises delivering a capped mRNA containing the open reading frame encoding the retrotransposase. In some embodiments, delivery of the engineered retrotransposase system to the target nucleic acid locus comprises delivering a translated polypeptide. In some embodiments, delivery of the engineered retrotransposase system to the target nucleic acid locus comprises delivering a deoxyribonucleic acid (DNA) encoding the engineered guide RNA operably linked to a ribonucleic acid (RNA) pol III promoter.

In some embodiments, the retrotransposase does not induce a break at or proximal to said target nucleic acid locus.

In one aspect, the present disclosure provides a host cell comprising an open reading frame encoding a heterologous retrotransposase. In some embodiments, the retrotransposase comprises a sequence having at least about 70% sequence identity to any one of SEQ ID NOs: 1-29, 393-735, or 799-895. In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895. In some embodiments, the retrotransposase may be substantially identical to any one of SEQ ID NOs: 1-29, 393-735, or 799-895.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a documented retrotransposase.

In some embodiments, the cargo nucleotide sequence is flanked by a 3′ untranslated region (UTR) and a 5′ untranslated region (UTR).

In some embodiments, the retrotransposase is configured to transpose the cargo nucleotide sequence as double-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the host cell is an E. coli cell. In some embodiments, the E. coli cell is a λDE3 lysogen or the E. coli cell is a BL21(DE3) strain. In some embodiments, the E. coli cell has an ompT lon genotype.

In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof.

In some embodiments, the open reading frame comprises a sequence encoding an affinity tag linked in-frame to a sequence encoding the retrotransposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the retrotransposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof.

In some embodiments, the open reading frame is codon-optimized for expression in the host cell. In some embodiments, the open reading frame is provided on a vector. In some embodiments, the open reading frame is integrated into a genome of the host cell.

In one aspect, the present disclosure provides a culture comprising a host cell described herein in compatible liquid medium.

In one aspect, the present disclosure provides a method of producing a retrotransposase, comprising cultivating a host cell described herein in compatible growth medium. In some embodiments, the method further comprises inducing expression of the retrotransposase by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the retrotransposase. In some embodiments, the IMAC affinity tag is linked in-frame to the sequence encoding the retrotransposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the retrotransposase. In some embodiments, the method further comprises performing subtractive IMAC affinity chromatography to remove the affinity tag from a composition comprising the retrotransposase.

In one aspect, the present disclosure provides a method of disrupting a locus in a cell. In some embodiments, the method comprises contacting to the cell a composition comprising a retrotransposase. In some embodiments, the retrotransposase has at least equivalent transposition activity to a documented retrotransposase in a cell. In some embodiments, the retrotransposase comprises a sequence having at least about 70% sequence identity to any one of SEQ ID NOs: 1-29, 393-735, or 799-895. In some embodiments, the retrotransposase comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-29, 393-735, or 799-895. In some embodiments, the retrotransposase may be substantially identical to any one of SEQ ID NOs: 1-29, 393-735, or 799-895.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a documented retrotransposase.

In some embodiments, the cargo nucleotide sequence is flanked by a 3′ untranslated region (UTR) and a 5′ untranslated region (UTR).

In some embodiments, the retrotransposase is configured to transpose the cargo nucleotide sequence as double-stranded deoxyribonucleic acid polynucleotide. In some embodiments, the retrotransposase is configured to transpose the cargo nucleotide sequence as single-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase comprises a sequence complementary to a eukaryotic, fungal, plant, mammalian, or human genomic polynucleotide sequence. In some embodiments, the retrotransposase comprises a sequence complementary to a eukaryotic genomic polynucleotide sequence. In some embodiments, the retrotransposase comprises a sequence complementary to a fungal genomic polynucleotide sequence. In some embodiments, the retrotransposase comprises a sequence complementary to a plant genomic polynucleotide sequence. In some embodiments, the retrotransposase comprises a sequence complementary to a mammalian genomic polynucleotide sequence. In some embodiments, the retrotransposase comprises a sequence complementary to a human genomic polynucleotide sequence.

In some embodiments, the retrotransposase may comprise a variant having one or more nuclear localization sequences (NLSs). The NLS may be proximal to the N- or C-terminus of the retrotransposase. The NLS may be appended N-terminal or C-terminal to any one of SEQ ID NOs: 896-911, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 896-911. In some embodiments, the NLS may comprise a sequence substantially identical to any one of SEQ ID NOs: 896-911. In some embodiments, the NLS may comprise a sequence substantially identical to SEQ ID NO: 896. In some embodiments, the NLS may comprise a sequence substantially identical to SEQ ID NO: 897.

In some embodiments, the transposition activity is measured in vitro by introducing the retrotransposase to cells comprising the target nucleic acid locus and detecting transposition of the target nucleic acid locus in the cells. In some embodiments, the composition comprises 20 pmoles or less of the retrotransposase. In some embodiments, the composition comprises 1 pmol or less of the retrotransposase.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### EXAMPLES

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### Example 1—a Method of Metagenomic Analysis for New Proteins

Metagenomic samples were collected from sediment, soil, and animals. Deoxyribonucleic acid (DNA) was extracted with a Zymobiomics DNA mini-prep kit and sequenced on an Illumina HiSeq® 2500. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on documented retrotransposase protein sequences to identify new retrotransposases. Novel retrotransposase proteins identified by the search were aligned to documented proteins to identify potential active sites. This metagenomic workflow resulted in the delineation of the MG140 family described herein.

### Example 2—Discovery of MG140 Family of Retrotransposases

Analysis of the data from the metagenomic analysis of Example 1 revealed a new cluster of undescribed putative retrotransposase systems comprising 1 family (MG140). The corresponding protein sequences for these new enzymes and their example subdomains are presented as SEQ ID NOs: 1-29, 393-401, and 799-894.

### Example 3—Integration of Reverse Transcribed DNA In Vitro Activity (Prophetic)

Integrase activity can be conducted via expression in an E. coli lysate-based expression system (for example, myTXTL, Arbor Biosciences). The components used for in vitro testing are three plasmids: an expression plasmid with the retrotransposon gene(s) under a T7 promoter, a target plasmid, and a donor plasmid which contains 5′ and 3′ UTR sequences recognized by the retrotransposase around a selection marker gene (e.g. Tet resistance gene). The lysate-based expression products, target DNA, and donor plasmid are incubated to allow for transposition to occur. Transposition is detected via PCR. In addition, the transposition product will be tagmented with T5 and sequenced via NGS to determine the insertion sites on a population of transposition events. Alternatively, the in vitro transposition products can be transformed into E. coli under antibiotic (e.g. Tet) selection, where growth occurs when the selection marker is stably inserted into a plasmid. Either single colonies or a population of E. coli can be sequenced to determine the insertion sites.

Integration efficiency can be measured via ddPCR or qPCR of the experimental output of target DNA with integrated cargo, normalized to the amount of unmodified target DNA also measured via ddPCR.

This assay may also be conducted with purified protein components rather than from lysate-based expression. In this case, the proteins are expressed in E. coli protease-deficient B strain under T7 inducible promoter, the cells are lysed using sonication, and the His-tagged protein of interest is purified using HisTrap FF (GE Lifescience) Ni-NTA affinity chromatography on the AKTA Avant FPLC (GE Lifescience). Purity is determined using densitometry in ImageLab software (Bio-Rad) of the protein bands resolved on SDS-PAGE and InstantBlue Ultrafast (Sigma-Aldrich)-57-oomassie stained acrylamide gels (Bio-Rad). The protein is desalted in storage buffer composed of 50 mM Tris-HCl, 300 mM NaCl, 1 mM TCEP, 5% glycerol; pH 7.5 (or other buffers as determined for maximum stability) and stored at −80° C. After purification the transposon gene(s) are added to the target DNA and donor plasmid as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 30-200 mM NaCl, 21 mM KCl, 1.35% glycerol, (measured pH 7.5) supplemented with 15 mM MgOAc₂.

### Example 4—Retrotransposon End Verification Via Gel Shift (Prophetic)

The retrotransposon ends are tested for retrotransposase binding via an electrophoretic mobility shift assay (EMSA). In this case, a target DNA fragment (100-500 bp) is end-labeled with FAM via PCR with FAM-labeled primers. The 3′ UTR RNA and 5′ UTR RNA are generated in vitro using T7 RNA polymerase and purified. The retrotransposase proteins are synthesized in an in vitro transcription/translation system (e.g. PURExpress). After synthesis, 1 pL of protein is added to 50 nM of the labeled DNA and 100 ng of the 3′ or 5′ UTR RNA in a 10 μL reaction in binding buffer (e.g. 20 mM HEPES pH 7.5, 2.5 mM Tris pH 7.5, 10 mM NaCl, 0.0625 mM EDTA, 5 mM TCEP, 0.005% BSA, 1 μg/mL poly(dI-dC), and 5% glycerol). The binding is incubated at 30° for 40 minutes, then 2 μL of 6× loading buffer (60 mM KCl, 10 mM Tris pH 7.6, 50% glycerol) is added. The binding reaction is separated on a 5% TBE gel and visualized. Shifts of the 3′ or 5′ UTR in the presence of retrotransposase protein and target DNA can be attributed to successful binding and are indicative of retrotransposase activity. This assay can also be performed with retrotransposase truncations or mutations, as well as using E. coli extract or purified protein.

### Example 5—Cleavage of Target DNA Verification (Prophetic)

To confirm that the retrotransposase is involved in cleavage of target DNA, short (˜140 bp) DNA fragments are labelled at both ends with FAM via PCR with FAM-labeled primers. In vitro transcription/translation retrotransposase products are pre-incubated with 1 pg of Rnase A (negative control), or 3′ UTR, 5′ UTR or non-specific RNA fragments (control), followed by incubating with labeled target DNA at 37° C. The DNA is then analyzed on a denaturing gel. Cleavage of one or both strands of DNA can result in labelled fragments of various sizes, which migrate at different rates on the gel.

### Example 6—Integrase Activity in E. coli (Prophetic)

Engineered E. coli strains are transformed with a plasmid expressing the retrotransposon genes and a plasmid containing a temperature-sensitive origin of replication with a selectable marker flanked by 5′ and 3′ UTR of the retrotransposon involved in integration. Transformants induced for expression of these genes are then screened for transfer of the marker to a genomic target by selection at restrictive temperature for plasmid replication and the marker integration in the genome is confirmed by PCR.

Integrations are screened using an unbiased approach. In brief, purified gDNA is tagmented with Tn5, and DNA of interest is then PCR amplified using primers specific to the Tn5 tagmentation and the selectable marker. The amplicons are then prepared for NGS sequencing. Analysis of the resulting sequences is trimmed of the transposon sequences and flanking sequences are mapped to the genome to determine insertion position, and insertion rates are determined.

### Example 7—Integration of Reverse Transcribed DNA into Mammalian Genomes (Prophetic)

To show targeting and cleavage activity in mammalian cells, the integrase proteins are purified in E. coli or sf9 cells with 2 NLS peptides either in the N, C or both terminus of the protein sequence. In this procedure, a plasmid containing a selectable neomycin resistance marker (NeoR), or a fluorescent marker flanked by the 5′ and 3′ UTR regions involved in transposition and under control of a CMV promoter is synthesized. Cells are be transfected with the plasmid, recovered for 4-6 hours for RNA transcription, and subsequently electroporated with purified integrase proteins. Antibiotic resistance integration into the genome is quantified by G418-resistant colony counts (selection to start 7 days post-transfection), and positive transposition by the fluorescent marker is assayed by fluorescence activated cell cytometry. 7-10 days after the second transfection, genomic DNA is extracted and used for the preparation of an NGS library. Off target frequency is assayed by fragmenting the genome and preparing amplicons of the transposon marker and flanking DNA for NGS library preparation. At least 40 different target sites are chosen for testing each targeting system's activity.

Integration in mammalian cells can also be assessed via RNA delivery. An RNA encoding the retrotransposase with 2 NLS is designed, and cap and polyA tail are added. A second RNA is designed containing a selectable neomycin resistance marker (NeoR) or a fluorescent marker flanked by the 5′ and 3′ UTR regions. The RNA constructs are introduced into mammalian cells via Lipofectamine™ RNAiMAX or TransIT®-mRNA transfection reagent. 10 days post-transfection, genomic DNA is extracted to measure transposition efficiency using ddPCR and NGS.

### Example 8—Bioinformatic Discovery of RTs

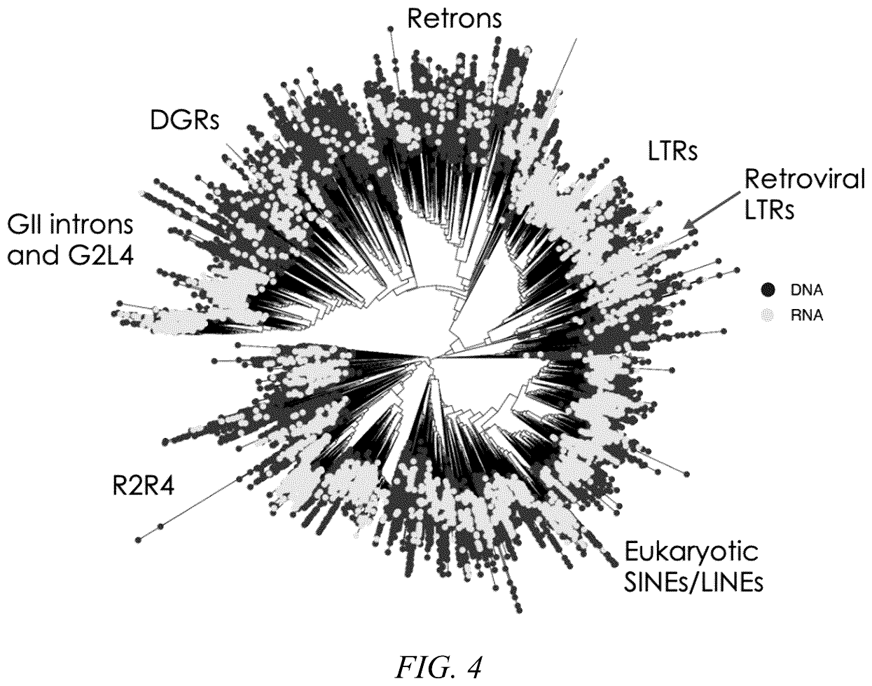

An extensive assembly-driven metagenomic database of microbial, viral, and eukaryotic genomes was mined to retrieve predicted proteins with reverse transcriptase function. Over 4.5 million RT proteins were predicted on the basis of having a hit to the Pfam domains PF00078 and PF07727, of which 3.4 million had a significant e-value (<1×10⁻⁵). After filtering for complete ORFs with an RT (reverse transcriptase) domain coverage of ≥70%, and with predicted catalytic residues ([F/Y]XDD), nearly half a million proteins were retained for further analysis. The RT domains were extracted from this set of proteins, as well as from reference sequences retrieved from public databases. The domain sequences were clustered at 50% identity over 80% coverage with Mmseqs2 easy-cluster (see Bioinformatics 2016 May 1; 32(9):1323-30, which is incorporated by reference in its entirety herein), representative sequences (26,824 in total) were aligned with MAFFT with parameters—globalpair—large (see Bioinformatics 2016; 32: 3246-3251, which is incorporated by reference in its entirety herein), and the domain alignment was used to infer a phylogenetic tree with FastTree2 (see Plos One 2010; 5: e9490, which is incorporated by reference in its entirety herein). Phylogenetic analysis of RT domains suggest that many different classes of RTs with high sequence diversity were recovered (FIG. 4).

### Example 9—Example Non-LTR Retrotransposons (MG140, MG146, MG147, MG148, and MG149 Families)

### Retrotransposon Bioinformatic Analysis

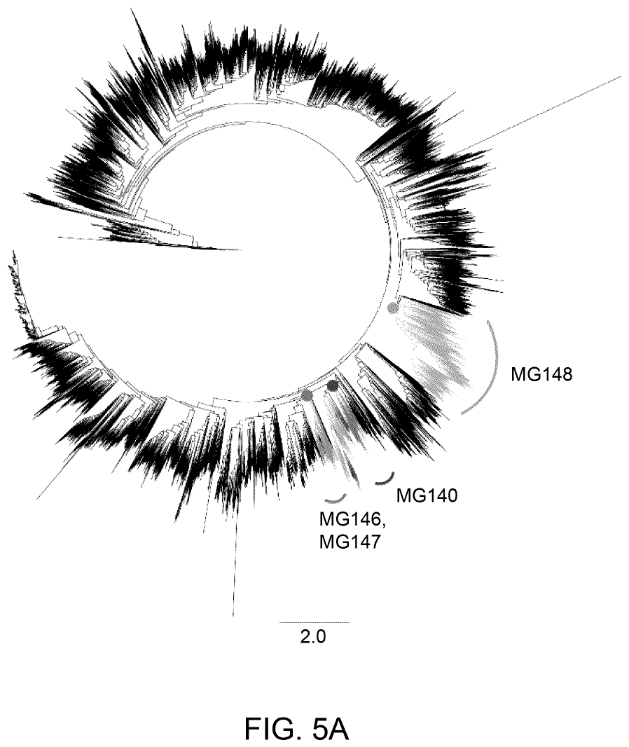

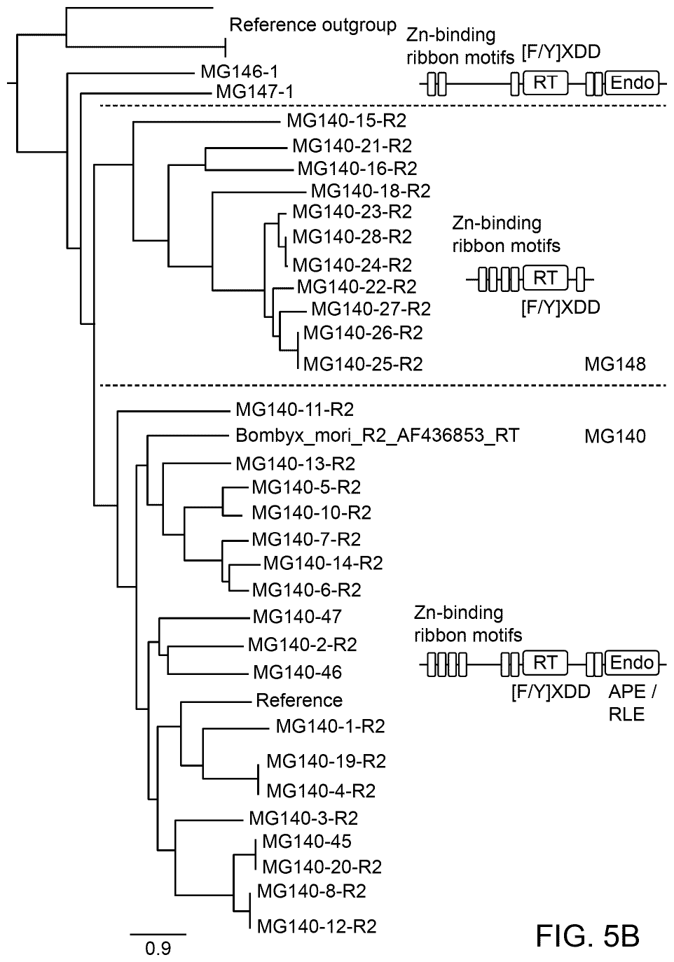

Non long terminal repeat (non-LTR) retrotransposases are capable of integrating large cargo into a target site via reverse transcription of an RNA template. Non-LTR retrotransposases were identified within the R2/R4 and LINE clades from the phylogenetic tree in FIG. 4. Full-length proteins containing RT domains classified as R2, R4, and LINEs were clustered at 99% sequence identity, and representative sequences were aligned with MAFFT with parameters—globalpair—large. A phylogenetic tree was inferred from this alignment and R2/R4 retrotransposase families, as well as other RT-related families, were delineated (FIG. 5A).

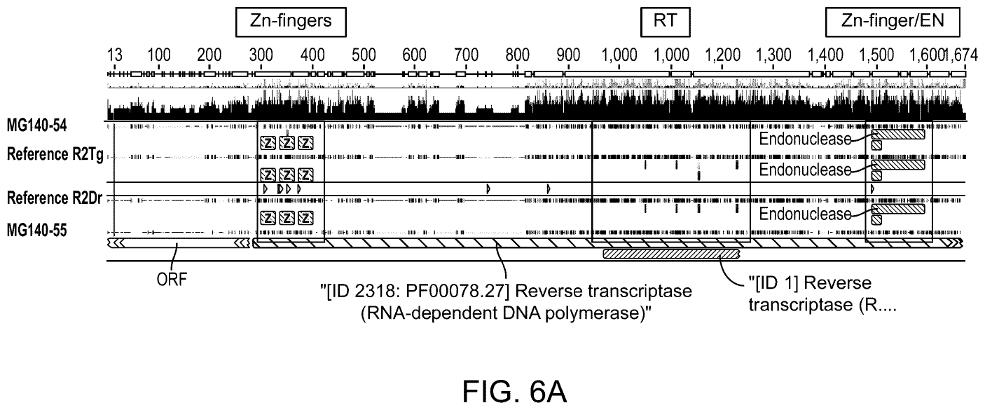

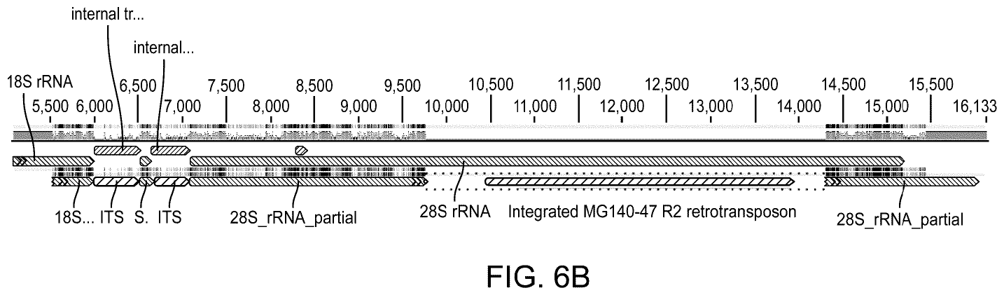

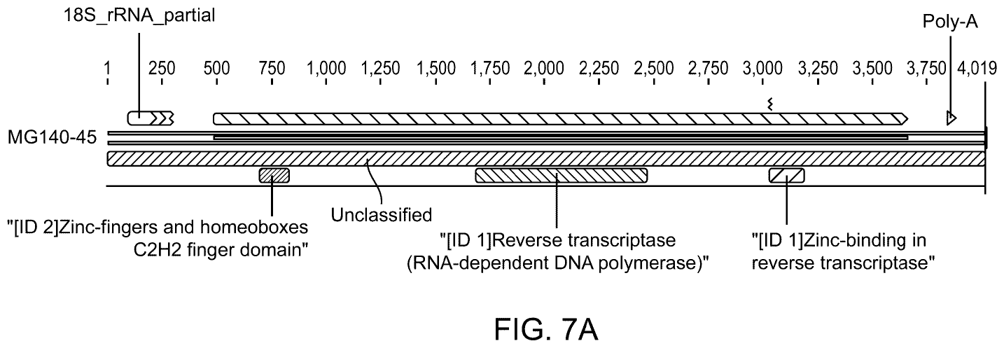

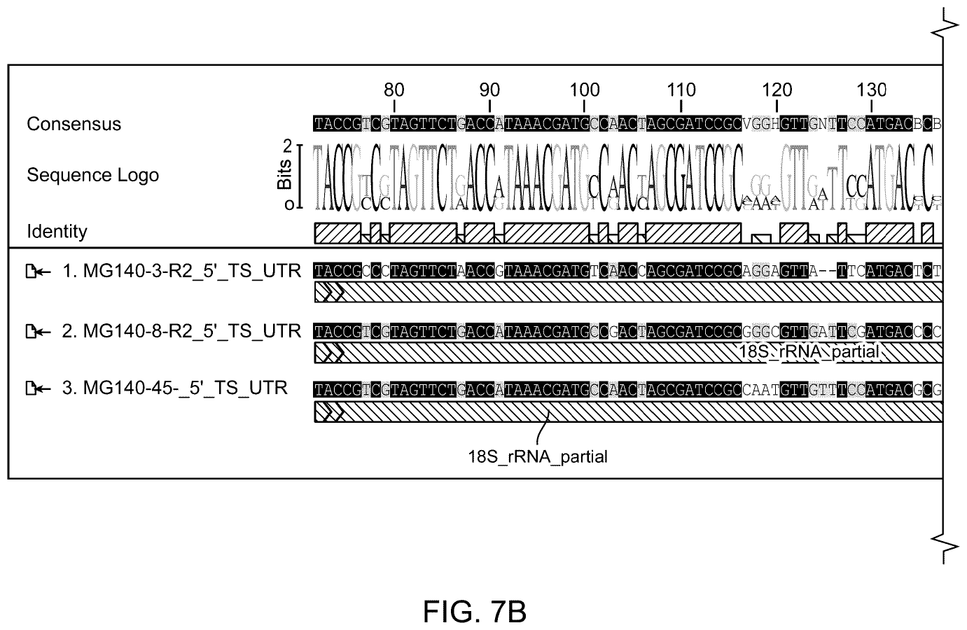

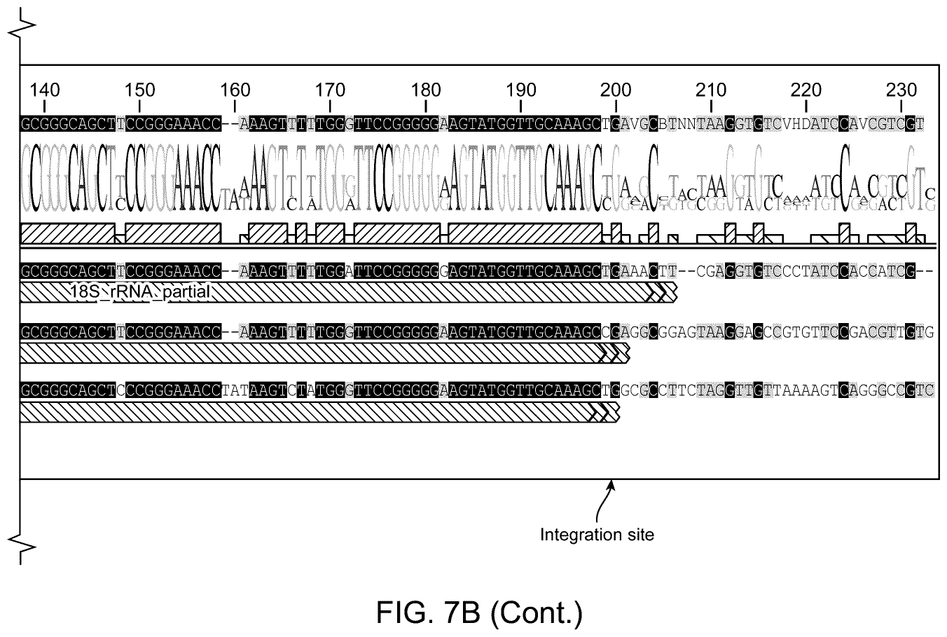

R2s are non-LTR retrotransposons that integrate cargo via target-primed reverse transcription (TPRT). Many R2 enzymes of the MG140 family contain an RT domain, as well as endonuclease domain and multiple Zn-binding ribbon motifs that delineate Zn-Fingers (FIGS. 5B and 6A). Some R2 retrotransposons integrate into the 28S rDNA, as shown by the boundaries of the MG140-47 (SEQ ID NO: 395) R2 retrotransposon flanked by fragments of a 28S rDNA gene (FIG. 6B). Other retrotransposons integrate into the 18S rRNA gene and contain a polyA or polyT tail that defines the 3′ end of the transposon (FIG. 7). It is possible that the exact target binding site, as well as 5′-UTR, 3′-UTR, and poly-T are involved in accurate and specific integration.

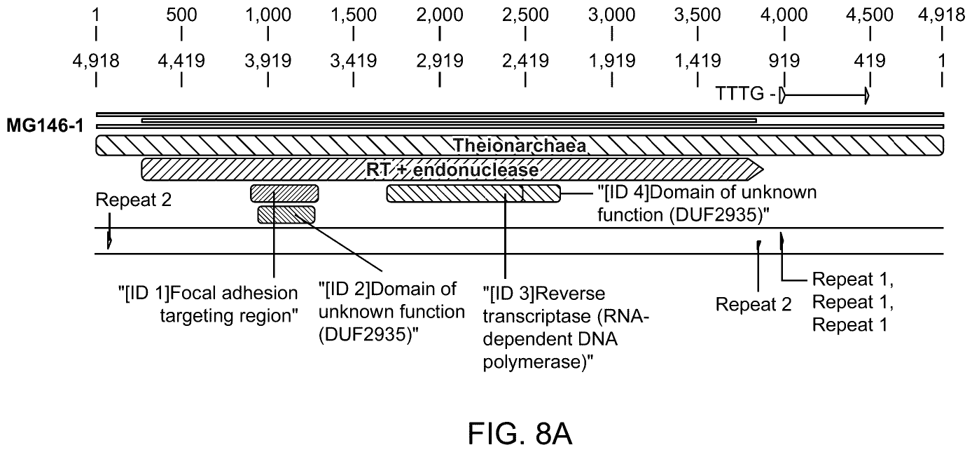

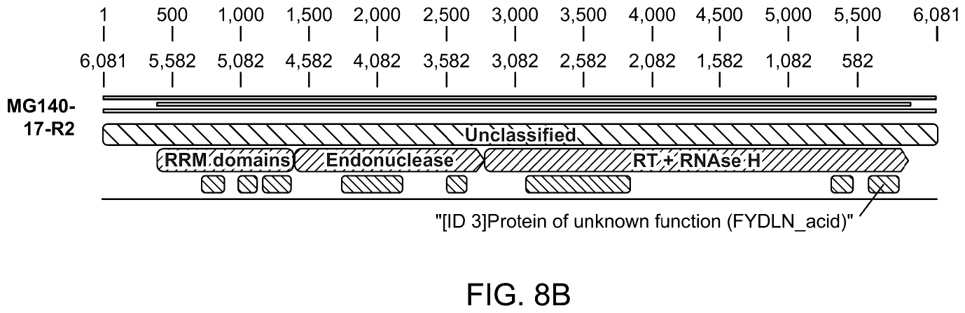

The retrotransposon MG146-1 (SEQ ID NO: 402), which was derived from an Archaeal genome, contains an RT domain, Zn-binding ribbon motifs, and an endonuclease domain, and the domain architecture within the enzyme differs from that of other single ORF non-LTR retrotransposons (FIG. 8A).

MG147 family member MG140-17-R2 (SEQ ID NO: 18) retrotransposon is organized into three ORFs flanked by 5′ and 3′ UTRs (FIG. 8B). The RNA recognition motif (RRM) gene is likely involved in recognition of the RNA template, while the endonuclease gene is likely involved in recognition and nicking of the target site. ORF three is the enzyme responsible for reverse transcription of the template and contains an RT domain, Zn-binding ribbon motifs, and an RNAse-H domain.

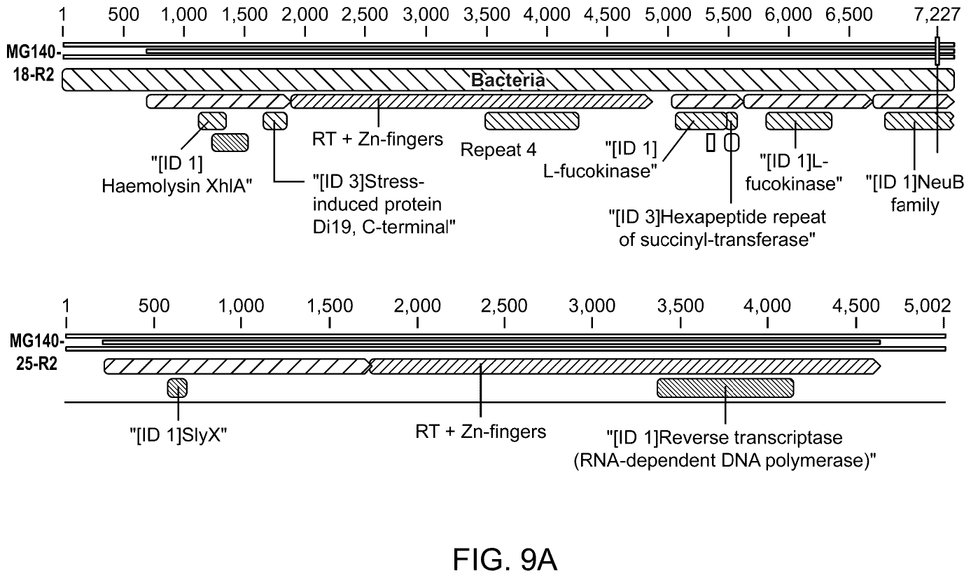

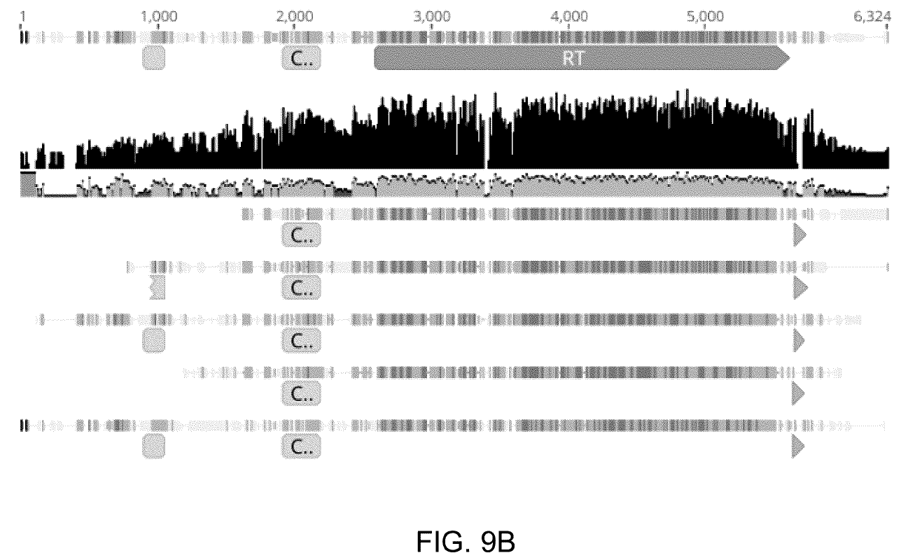

Family MG148 includes extremely divergent RT homologs, predicted to be active by the presence of all expected catalytic residues. Alignment at the nucleotide level for several family members uncovered conserved regions within the 5′ UTR, which are possibly involved in RT function, activity or mobilization (FIG. 9B).

Testing the In Vitro Activity of Retrotransposon RTs (Reverse Transcriptases) by qPCR

The in vitro activity of retrotransposon RTs was assessed by a primer extension reaction containing RT enzyme derived from a cell-free expression system (PURExpress, NEB) and 100 nM of RNA template (200 nt) annealed to a DNA primer in reaction buffer containing 40 mM Tris-HCl (pH 7.5), 0.2 M NaCl, 10 mM MgCl₂, 1 mM TCEP, and 0.5 mM dNTPs. The resulting full-length cDNA product was quantified by qPCR by extrapolating values from a standard curve generated with the DNA template of specific concentrations.

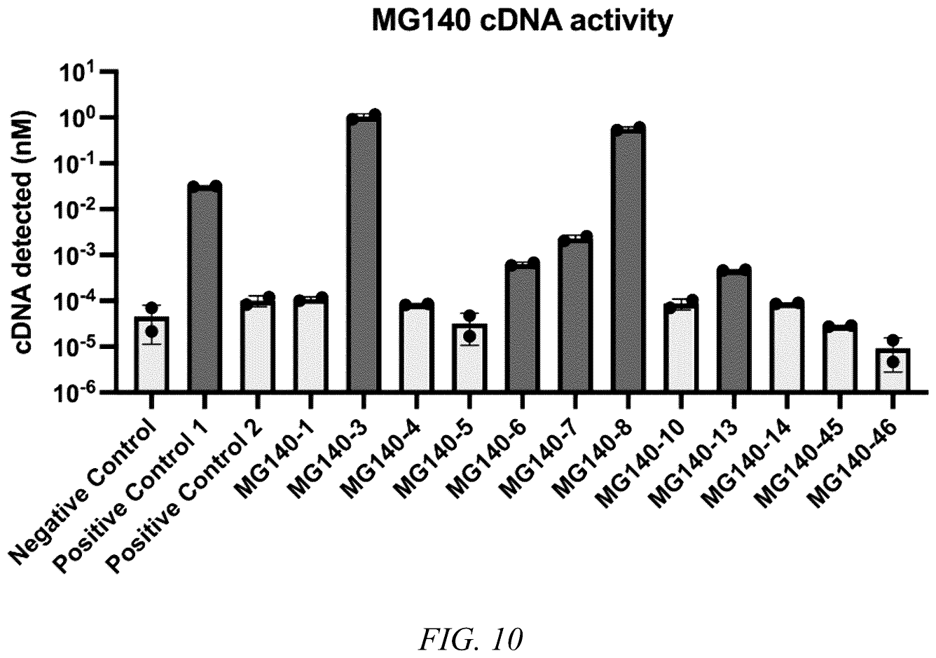

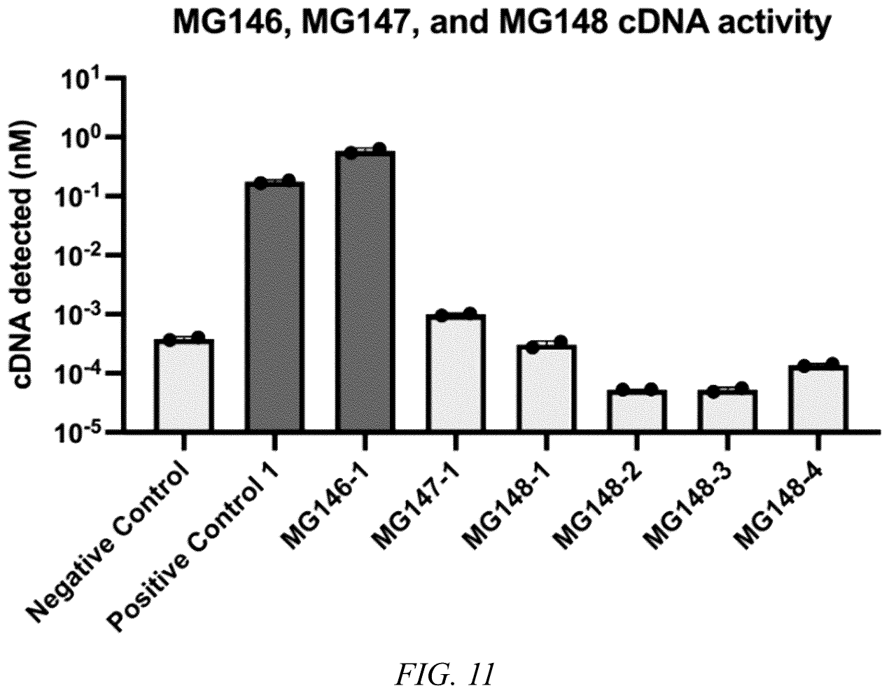

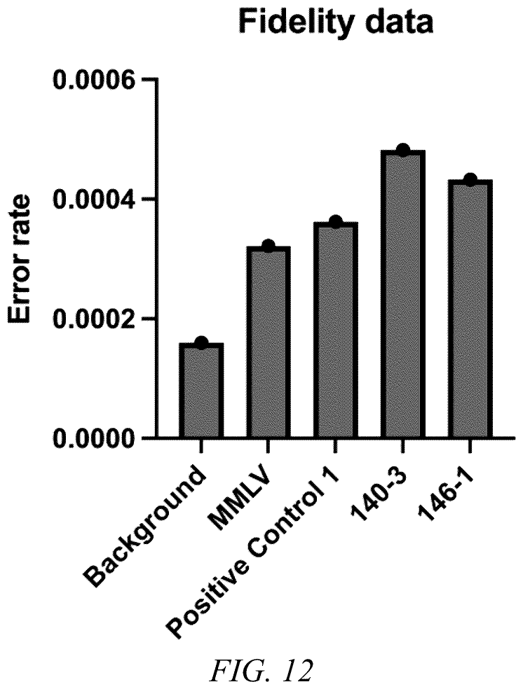

MG140-3 (SEQ ID NO: 3), MG140-6 (SEQ ID NO: 6), MG140-7 (SEQ ID NO: 7), MG140-8 (SEQ ID NO: 8), MG140-13 (SEQ ID NO: 14), and MG146-1 (SEQ ID NO: 402) are active via primer extension (FIGS. 10 and 11). Preliminary assessment of fidelity was performed for MG140-3 and MG146-1, resulting in a relative error rate 1.5 and 1.35-times higher than MMLV, respectively (FIG. 12). For fidelity measurements, the resulting full-length cDNA product generated in the primer extension assay described above was PCR-amplified, library-prepped, and subjected to next generation sequencing. Trimmed reads were aligned to the reference sequence and the frequency of misincorporation was calculated.

### Integration Site

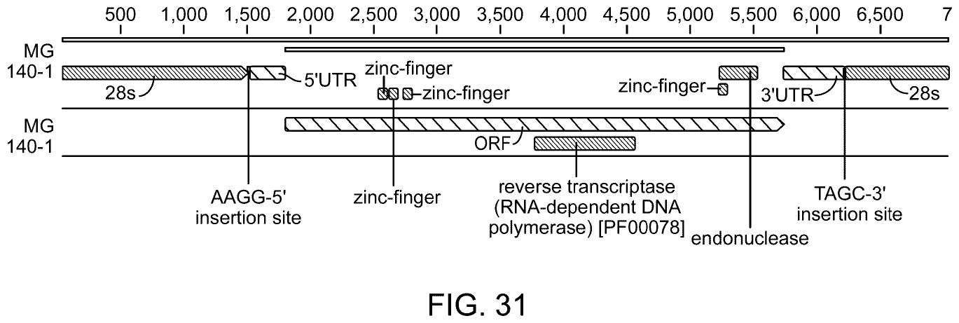

Some non-LTR retrotransposons (e.g. MG140 family such as MG140-1) are predicted to integrate into the 28S rDNA gene by targeting specific GGTGAC motifs, with the insertion site between the second (G) and third (T) positions. The N-terminus of such retrotransposon proteins contains three zinc (Zn) fingers (two of the CCHH type and one of type CCHC), which are followed by the reverse transcriptase (RT) domain with a YADD active site. The C-terminus of such retrotransposon proteins includes an endonuclease domain with an additional CCHC Zn-finger. The protein is flanked by 5′ and 3′ UTRs that are 289 and 478 bp long, respectively (FIG. 31).

### Example 10—Group II Intron RTs (MG153, MG163, MG164, MG165, MG166, MG167, MG168, MG169, and MG170 Families)

### Group II Bioinformatic Analysis

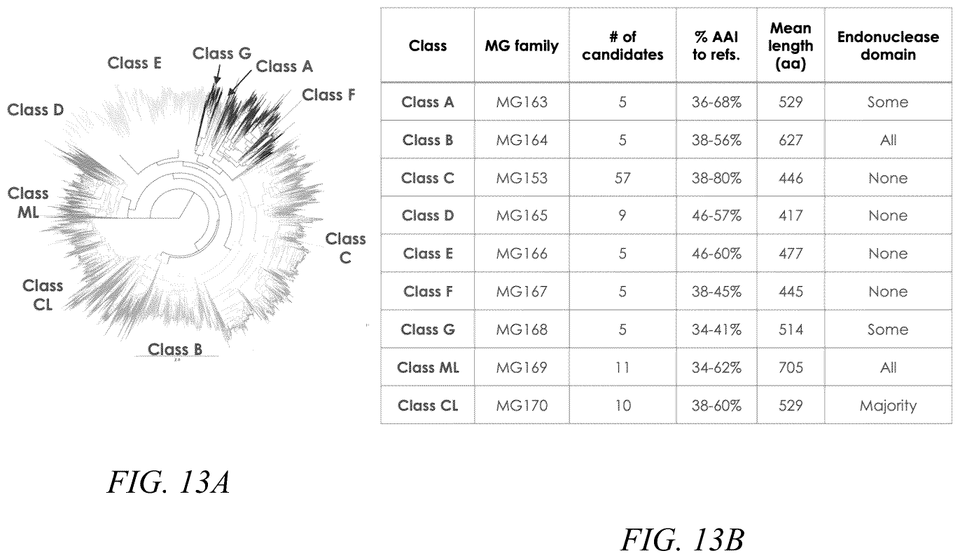

Group II introns are capable of integrating large cargo into a target site via reverse transcription of an RNA template. RT domains from Group II introns were identified and delineated in the phylogenetic tree in FIG. 4. Over 10,000 unique full-length Group II intron proteins containing RT domains from contigs with >2 kb of sequence flanking the RT enzyme were aligned with MAFFT with parameters—globalpair—large. A phylogenetic tree was inferred from this alignment and Group II intron families were further identified (FIG. 13). Group II intron enzymes can be classified into classes A-G, ML, and CL, and their domain architecture includes an RT domain predicted to be active, as well as a maturase domain involved in intron mobilization. Some Group II intron proteins contain an additional endonuclease domain likely involved in target recognition and cleavage. Many candidates from all families identified were nominated for laboratory characterization.

### Testing the In Vitro Activity of Group H Intron RTs Class C, D, and F

The in vitro activity of GII intron Class C (MG153), Class D (MG165), and Class F (MG167) RTs was assessed by a primer extension reaction containing RT enzyme derived from a cell-free expression system (PURExpress, NEB). Expression constructs were codon-optimized for E. coli and contained an N-terminal single Strep tag. Expression of the RT was confirmed by SDS-PAGE analysis. The substrate for the reaction was 100 nM of RNA template (200 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH (NEB), followed by the addition of 2×RNA loading dye (NEB). The resulting cDNA product(s) were separated on a 10% denaturing polyacrylamide gel and were visualized using a ChemiDoc on the Gel Green setting. RT activity was also assessed by qPCR with primers that amplify the full-length cDNA product. Products from the primer extension assay were diluted to ensure cDNA concentrations were within the linear range of detection. The amount of cDNA was quantified by extrapolating values from a standard curve generated with the DNA template of specific concentrations.

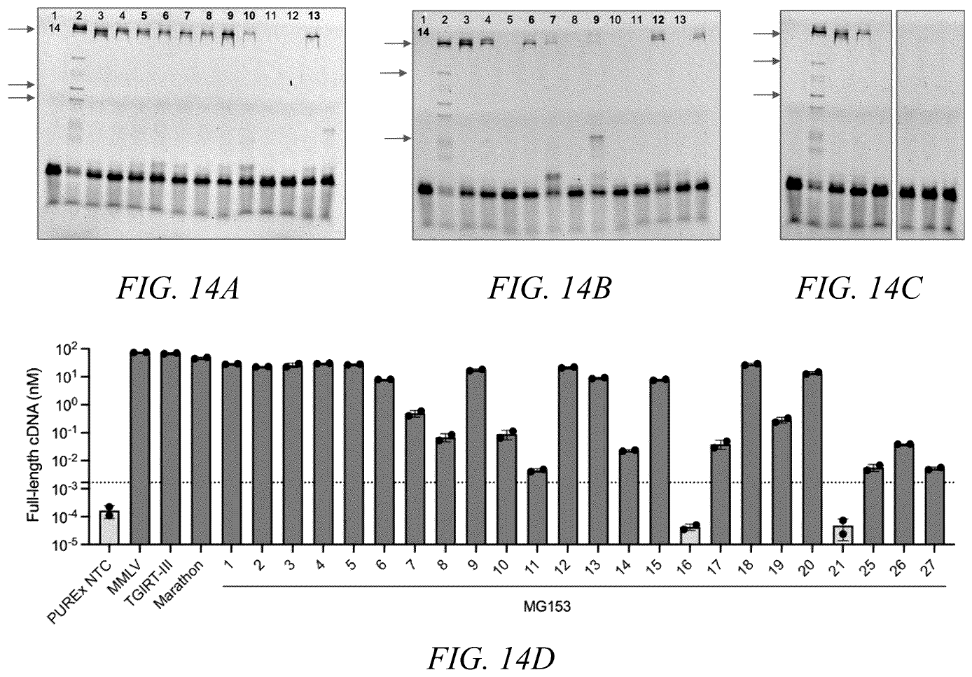

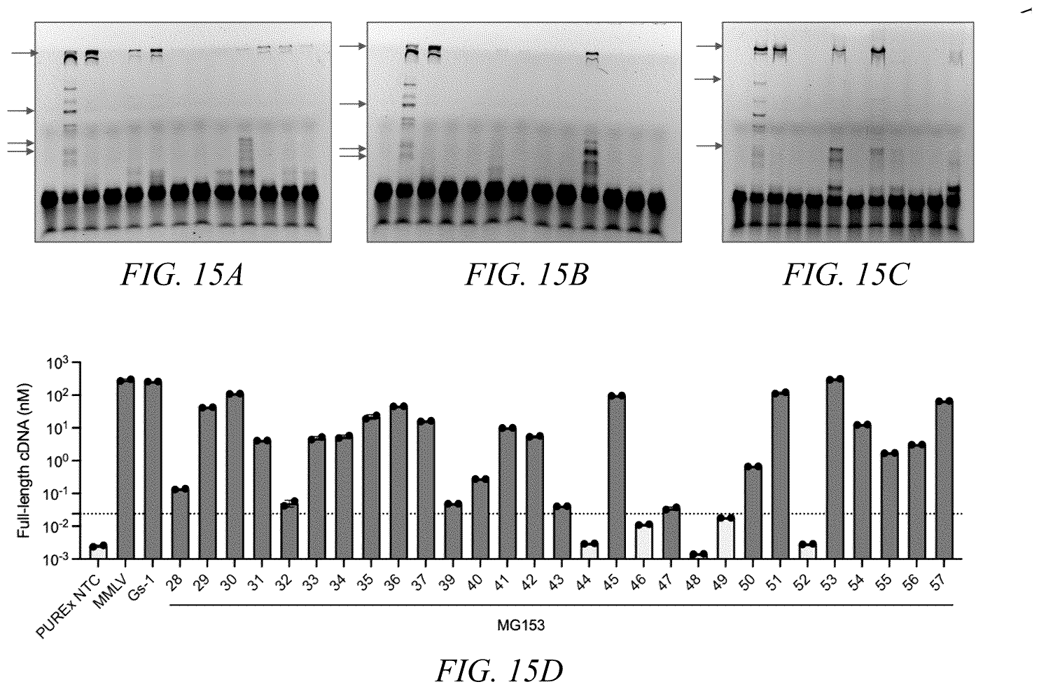

By detection of cDNA products on a denaturing gel, the following GII intron class C candidates were active under these experimental conditions: MG153-1 through MG153-6 (SEQ ID NOs: 555-560), MG153-9 (SEQ ID NO: 563), MG153-10 (SEQ ID NO: 564), MG153-12 (SEQ ID NO: 566), MG153-13 (SEQ ID NO: 567), MG153-15 (SEQ ID NO: 569), MG153-18 (SEQ ID NO: 572), MG153-20 (SEQ ID NO: 574), MG153-29 through MG153-31 (SEQ ID NOs: 580-582), MG153-33 through MG153-37 (SEQ ID NOs: 584-588), MG153-41 (SEQ ID NO: 592), MG153-42 (SEQ ID NO: 593), MG153-45 (SEQ ID NO: 596), MG153-51 (SEQ ID NO: 602), MG153-53 (SEQ ID NO: 604), MG153-54 (SEQ ID NO: 605), and MG153-57 (SEQ ID NO: 608). (FIGS. 14 and 15). Active novel candidates exhibit a varying degree of apparent processivity compared to the highly processive control GII Class C RTs GsI-IIC and MarathonRT, indicated by the presence of smaller cDNA drop-off products. By qPCR, the following additional candidates are also active under these experimental conditions (cDNA detected>10-fold above background): MG153-7 (SEQ ID NO: 561), MG153-8 (SEQ ID NO: 562), MG153-10 (SEQ ID NO: 564), MG153-11 (SEQ ID NO: 565), MG153-14 (SEQ ID NO: 568), MG153-17 (SEQ ID NO: 571), MG153-19 (SEQ ID NO: 573), MG153-25 through MG153-28 (SEQ ID NOs: 576-579), MG153-32 (SEQ ID NO: 583), MG153-39 (SEQ ID NO: 590), MG153-40 (SEQ ID NO: 591), MG153-43 (SEQ ID NO: 594), MG153-47 (SEQ ID NO: 598), MG153-50 (SEQ ID NO: 601), MG153-55 (SEQ ID NO: 606) and MG153-56 (SEQ ID NO: 607) (FIGS. 14D and 15D).

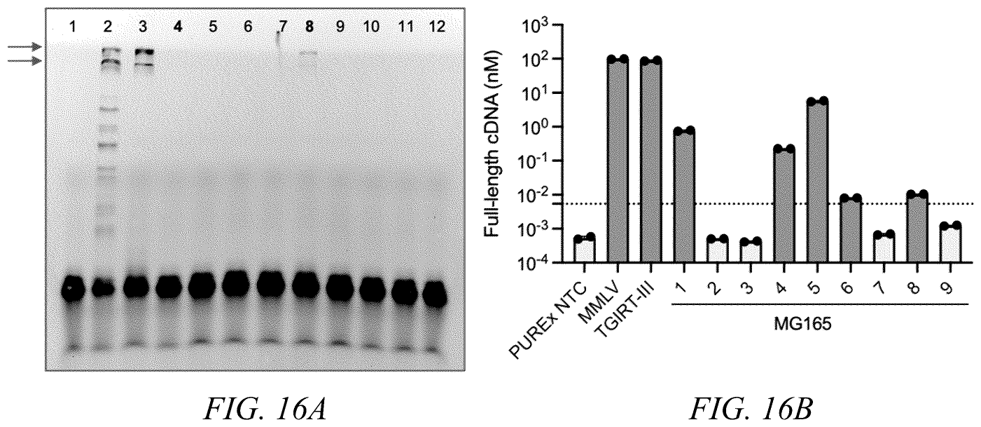

By detection of cDNA products on a denaturing gel, GII intron class D candidates MG165-1 (SEQ ID NO: 684) and MG165-5 (SEQ ID NO: 688) are active under these experimental conditions (FIG. 16A). By qPCR, additional candidates MG165-4 (SEQ ID NO: 687), MG165-6 (SEQ ID NO: 689), and MG165-8 (SEQ ID NO: 691) are also active under these experimental conditions (cDNA detected>10-fold above background) (FIG. 16B).

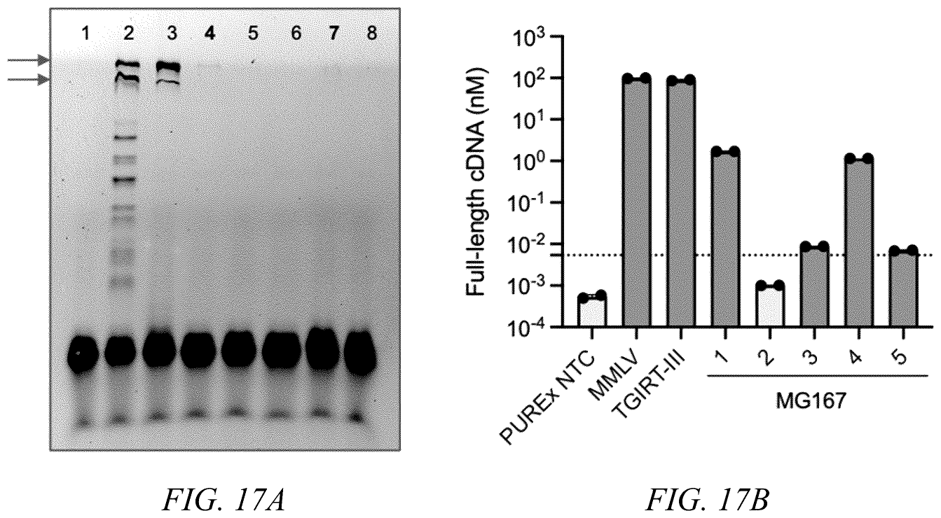

By detection of cDNA products on a denaturing gel, GII intron Class F candidates MG167-1 (SEQ ID NO: 698) and MG167-4 (SEQ ID NO: 701) are active under these experimental conditions (FIG. 17A). By qPCR, additional candidates MG167-3 (SEQ ID NO: 700) and MG167-5 (SEQ ID NO: 702) are also active under these experimental conditions (cDNA detected>10-fold above background) (FIG. 17B).

### Assessment of Relative Fidelity of GII Intron RTs

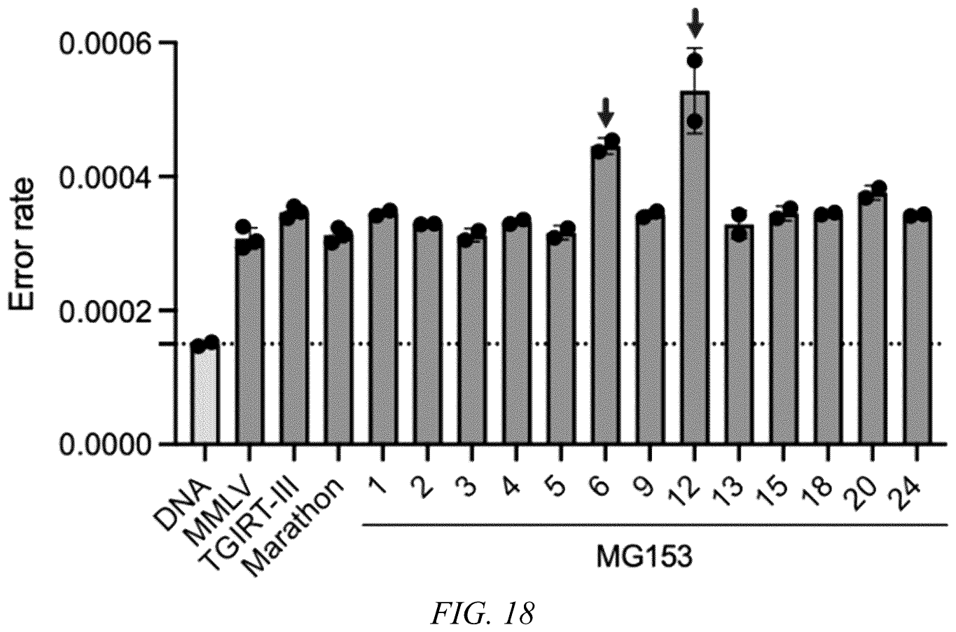

To assess the relative fidelity of GII Class C MG153 candidates, the resulting full-length cDNA product generated in the primer extension assay described above was PCR-amplified, library-prepped, and subjected to next generation sequencing. Paired reads were merged using bbmerge.sh requiring a perfect overlap and trimming all non-overlapping portions (Plos One 2017; 12: e0185056). Merged reads were then aligned to the reference template using BWA-MEM (Li H. 2013), and pysamstats (https://github.com/alimanfoo/pysamstats) was used to calculate the number of mismatches at each position relative to the reference. Of the GII Class C candidates tested, MG153-6 (SEQ ID NO: 560) and MG153-12 (SEQ ID NO: 566) have reproducibly higher error rates compared to MMLV control RT and other GII intron Class C RTs (FIG. 18).

Human Cells cDNA Synthesis Results

The ability of these enzymes to produce cDNA in a mammalian environment was tested by expressing them in mammalian cells and detecting cDNA synthesis by PCR, followed by agarose electrophoresis and D1000 TapeStation. Reverse transcriptases were cloned in a plasmid for mammalian expression under the CMV promoter as fusion proteins having MS2 coat protein (MCP) at the N terminus, in addition to a flag-HA tag (FH). MCP is a protein derived from the MS2 bacteriophage that recognizes a 20 nucleotide RNA stem loop with high affinity (subnanomolar Kd). By fusing the RTs with MCP and having the MS2 loops in the RNA template, it is ensured that once the RT is translated, it finds the RNA template and starts cDNA synthesis from the DNA primer hybridized to the RNA template.

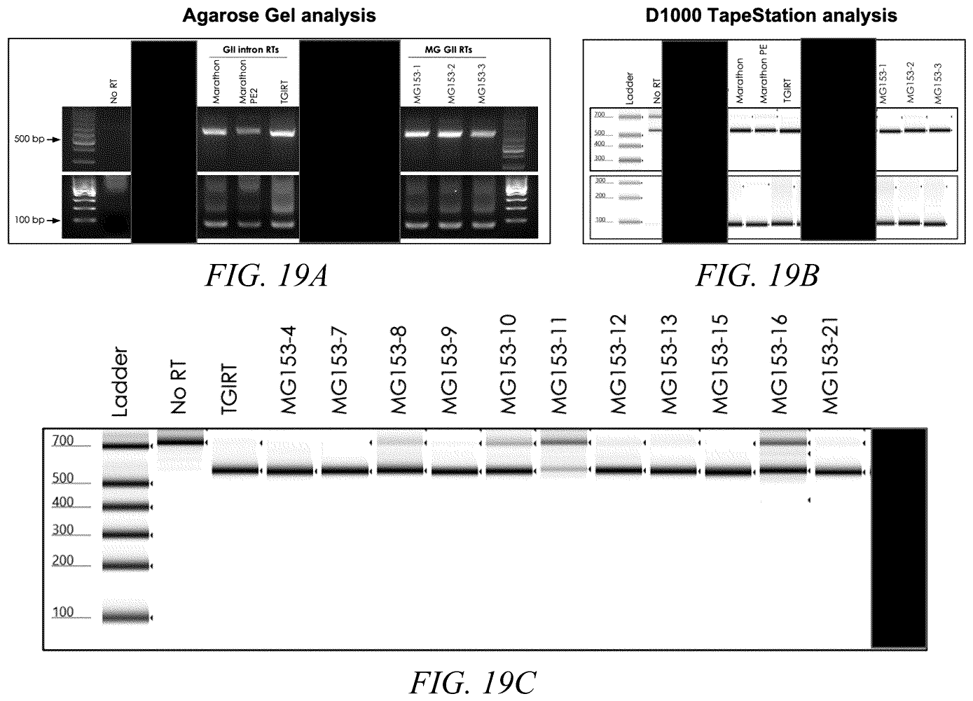

A plasmid containing MCP fused to the RT candidate under CMV promoter was cloned and isolated for transfection in HEK293T cells. Transfection was performed using lipofectamine 2000. mRNA codifying nanoluciferase (SEQ ID NO: 33) was made using mMESSAGE mMACHINE (Thermo Fisher) according to the manufacturer instructions. In order to degrade any DNA template left in the mRNA preparation, the reaction was treated with Turbo Dnase (Thermo Fisher) for 1 hour, and the mRNA was cleaned using MEGAclear Transcription Clean-Up kit (Thermo Fisher). The mRNA was hybridized to a complementary DNA primer (SEQ ID NO: 34) in 10 mM Tris pH 7.5, 50 mM NaCl at 95° C. for 2 min and cooled to 4° C. at the rate of 0.1° C./s. The mRNA/DNA hybrid was transfected into HEK293T cells using Lipofectamine Messenger Max 6 hours after the plasmid containing the MCP-RT fusion was transfected. 18 hours post mRNA/DNA transfection, cells were lysed using QuickExtra DNA Extraction Solution (Lucigen), 100 μL of quick extract was added per 24 well in a 24 well plate. The nanoluciferase is ˜500 bp long, primers to amplify products of 100 bp and 542 bp from the newly synthesized cDNA were designed (SEQ ID NOs: 38 and 39). cDNA was amplified using the set of primers mentioned above, and PCR products were detected by agarose gel electrophoresis (FIG. 19A) or DNA Tape Station (FIG. 19B).

Activity for the control GII intron RTs Marathon, Marathon PE2, and TGIRT was detected (FIGS. 19A and 19B), as shown by the presence of a 100 bp and 500 bp DNA product. Moreover, activity for novel GII intron derived RTs MG153-1 through MG153-4 (SEQ ID NOs: 555-558), MG153-7 through MG153-13 (SEQ ID NOs: 561-567), MG153-15 (SEQ ID NO: 569), MG153-16 (SEQ ID NO: 570) and MG153-21 (SEQ ID NO: 575) was also shown (FIGS. 19A, 19B, and 19C). The signal of the PCR product for the novel RTs was similar to that of Marathon and TGIRT. Altogether, this shows that these newly discovered RTs are expressed, fold properly, and are active inside living mammalian cells, opening options for their biotechnological applications.

Group II Intron RTs are Capable of Synthesizing cDNA Using Modified Primers

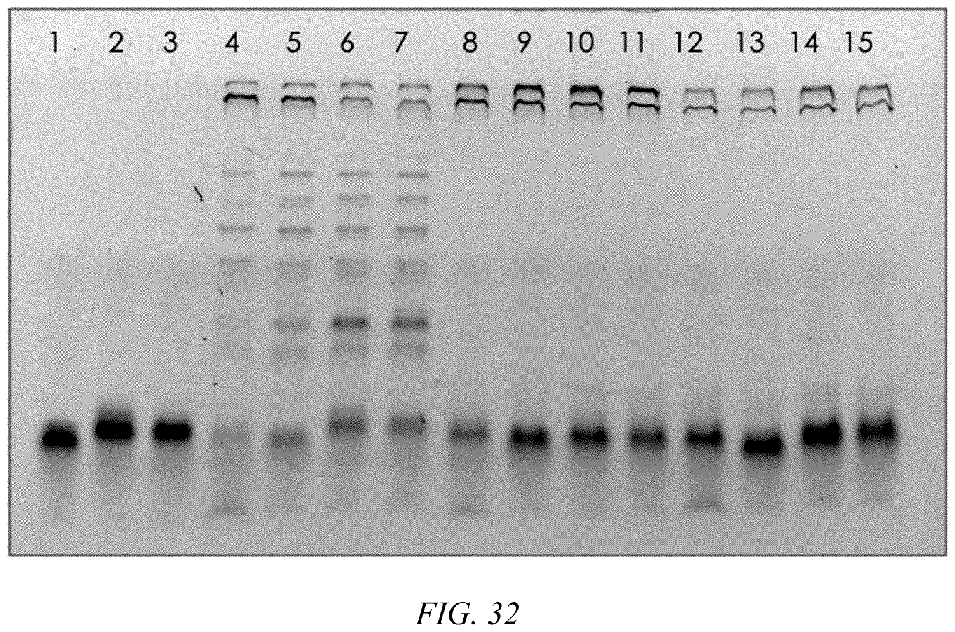

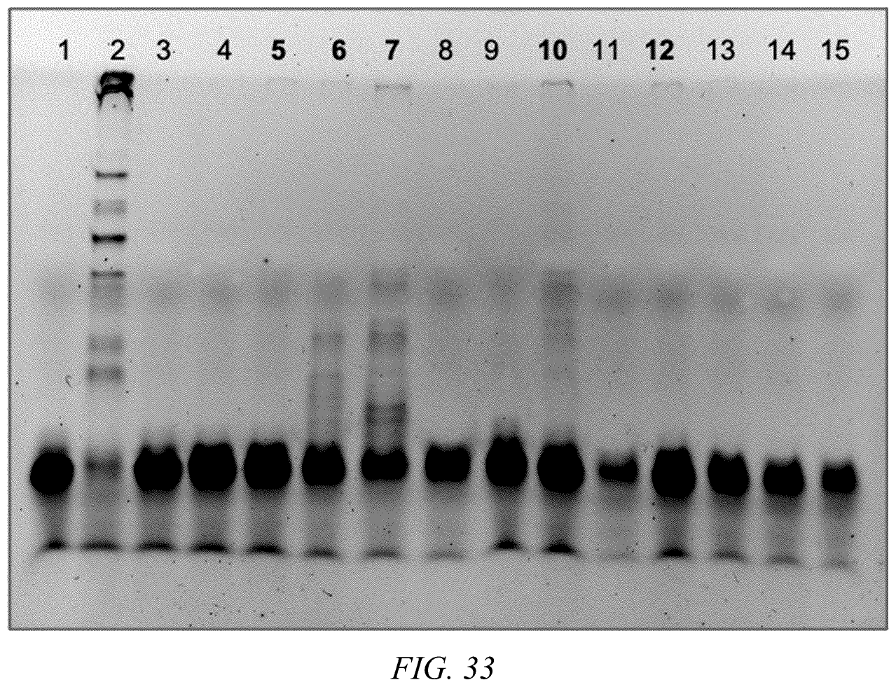

The in vitro activity of RTs was assessed by a primer extension reaction containing RT enzyme derived from a cell-free expression system (PURExpress, NEB). Expression constructs were codon-optimized for E. coli and contained an N-terminal single Strep tag. The substrate for the reaction was 100 nM of RNA template (202 nt) annealed to a 5′-FAM labeled DNA primer containing phosphorothioate (PS) bond modifications at various locations within the primer. Primer 1 (SEQ ID NO: 736, comprising a sequence/56-FAM/A*G*A*C*G*GTCACAGCTTGTCTG) contains 5 PS bonds at the 5′ end of the oligo. Primer 2 (SEQ ID NO: 737, comprising a sequence/56-FAM/A*G*A*C*G*GTCACAGCTT*G*T*C*T*G wherein * denotes a phosphorothioate bond) contains 5 PS bonds at both 5′ and 3 ends of the oligo. Primer 3 (SEQ ID NO: 738, comprising a sequence of/56-FAM/A*G*A*C*G*GTCACAGCTT*G*T*C*TG, wherein * denotes a phosphorothioate bond) differs from Primer 2 in that a standard bond is replaced between the two most 3′ terminal nucleotides. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH (NEB), followed by the addition of 2×RNA loading dye (NEB). The resulting cDNA product(s) were separated on a 10% denaturing polyacrylamide gel and were visualized using a ChemiDoc on the Gel Green setting. Based on these results, the control RTs MMLV (viral) and TGIRT-III (GII intron) are both capable of performing primer extension with all modified primers (FIG. 32). The GII intron RT MG153-9 is also capable of extending from all tested PS-modified DNA primers (FIG. 33).

Human Cells RT Expression and cDNA Synthesis Results

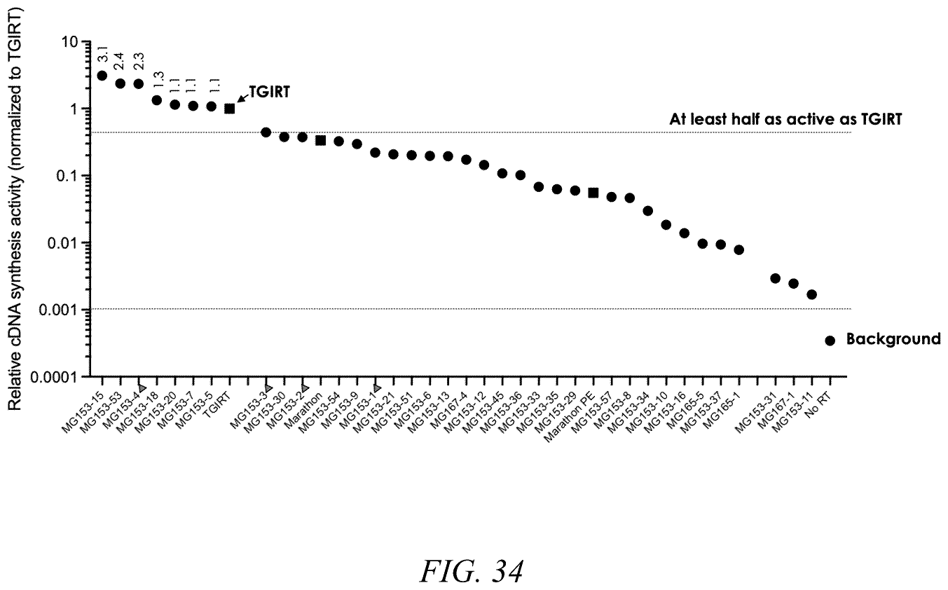

The ability of novel GII RTs to synthesize cDNA in a mammalian cell environment was tested as previously described with insubstantial modifications. cDNA synthesis was detected using PCR and analyzed by agarose gel electrophoresis or TapeStation. In order to have a quantitative readout, a Tagman qPCR assay was developed using Tagman qPCR primers already documented with a Tagman probe listed as SEQ ID NO: 739. All tested candidates of the MG153 family were active to various degrees, with activity as broad as four orders of magnitude (FIG. 34). RTs of families tested include MG153-1 through MG153-13, MG153-15, MG153-16, MG153-18, MG153-20, MG153-21, MG153-29 through MG153-31, MG153-33 through MG153-37, MG153-45, MG153-51, MG153-53, MG153-54, MG153-57, MG165-1, MG165-5, MG167-1 and MG167-4. Several RTs (MG153-15, MG153-53, MG153-4, MG153-18, MG153-20, MG153-7 and MG153-5) outperformed the TGIRT control (FIG. 34).

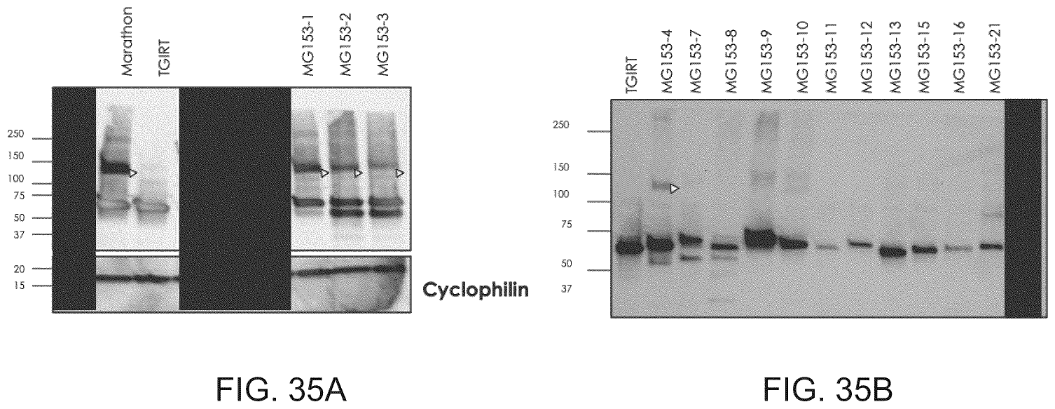

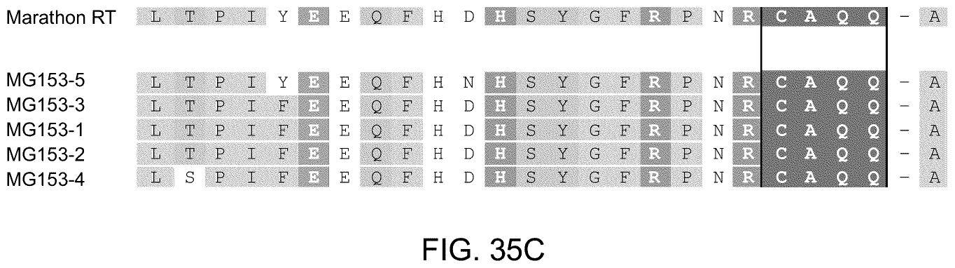

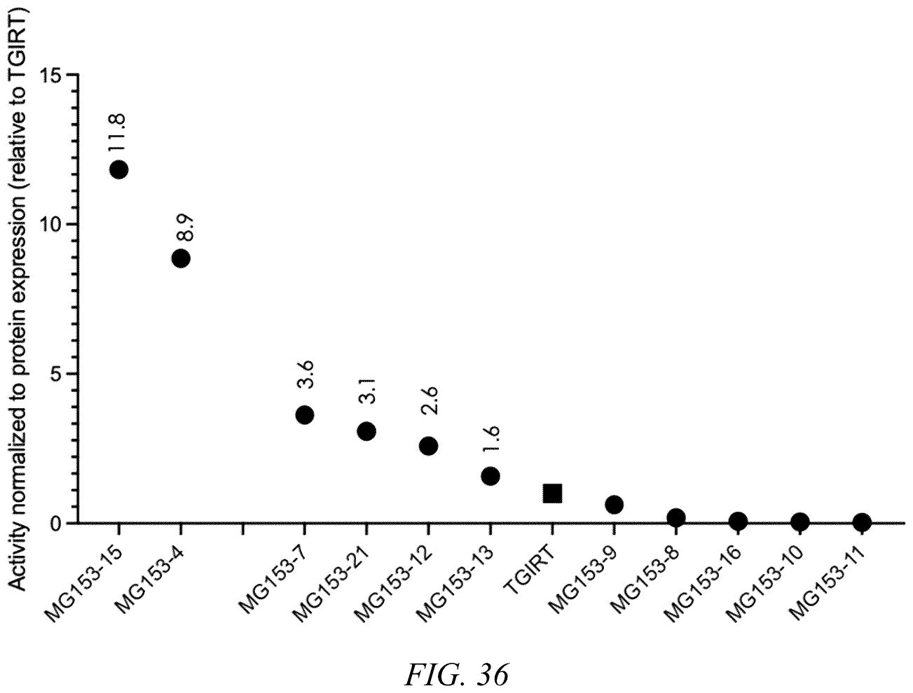

In order to understand protein expression and stability of the GII RTs in mammalian cells, immunoblots were performed. Briefly, transfected cells were lysed with RIPA lysis buffer (Thermo Fisher) supplemented with protease inhibitors (80 μL per well in a 24 well format). The lysate was centrifuged at 14,000 g for 10 min at 4° C. in order to remove insoluble aggregates. Proteins were quantified using BCA. 3 or 10 pg of total protein was loaded per lane in a 4-12% polyacrylamide SDS gel (Thermo Fisher). All lanes were normalized to the same amount of protein. Proteins were transferred to a PVDF membrane using the iBlot gel transfer system (Invitrogen). Proteins were detected by using a rabbit HA antibody (Cell Signaling), using an HRP-based detection method. Results suggest varying levels of protein expression or stability, as given by the intensity of the band (FIGS. 35A-35C). We quantified the expression of each protein and normalized cDNA synthesis activity to total protein expression: seven MG153 RTs outperformed the TGIRT control (FIG. 36). Remarkably MG153-15 shows 10-fold higher cDNA synthesis activity than TGIRT under these conditions.

Some GII derived RTs form very stable dimers, including one of the positive controls, MarathonRT, as well as MG153-1 through MG153-4 and MG153-9 (FIGS. 35A-35C). The “CAQQ” motif was documented as responsible for stable dimerization in Marathon RT (Nat Struct Mol Biol. 2016 June; 23(6): 558-565). RTs that showed stable dimer formation on immunoblots (MG153-1 through MG153-4) also contain the CAQQ dimerization amino acid motif (FIG. 35C). Dimerization may be an unfavorable feature due to added complexity, therefore RTs that do not form dimers may be optimal for specific biotechnological applications.

TABLE 2
Expected molecular sizes for tested RT candidates

| RT | Expected Protein Size (kDa)* |
|---|---|
| Marathon | 67.8 |
| TGIRT | 67 |
| MG153-1 | 74 |
| MG153-2 | 74 |
| MG153-3 | 74 |
| MG153-4 | 67.6 |
| MG153-7 | 71.7 |
| MG153-8 | 67.6 |
| MG153-9 | 72 |
| MG153-10 | 72.2 |
| MG153-11 | 70.9 |
| MG153-12 | 72.5 |
| MG153-13 | 67.9 |
| MG153-15 | 68.6 |
| MG153-16 | 71.7 |
| MG153-21 | 70.6 |

*Size includes a Flag-HA-MCP tag

### Example 11—G2L4 (MG172 Family)

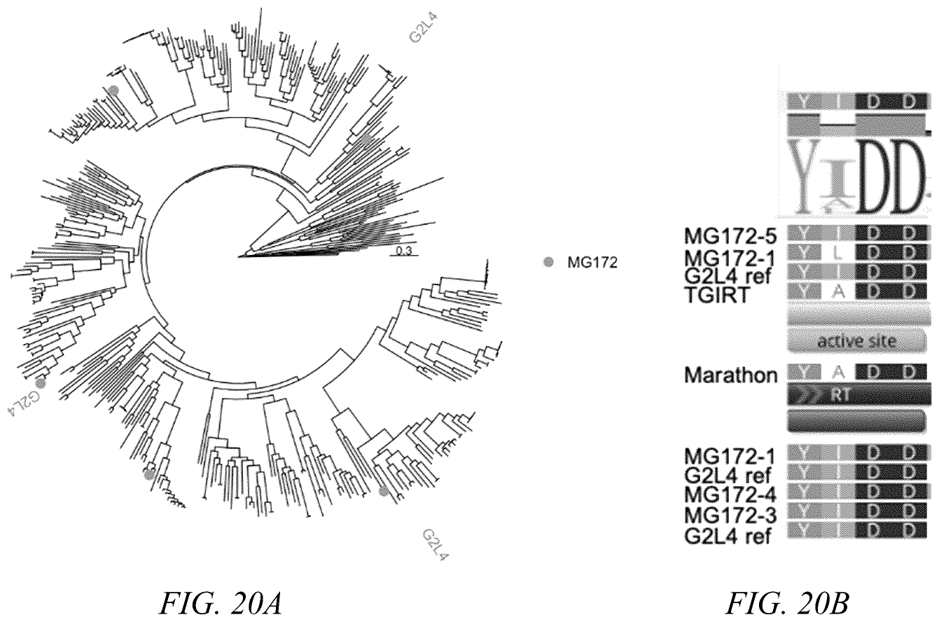

G2L4 are RT-containing sequences distantly related to Group II introns (Group II intron-like RTs), which were identified in FIG. 4. Over 600 novel full-length G2L4 enzymes were aligned with MAFFT with parameters—globalpair—large and a phylogenetic tree was inferred from this alignment (FIG. 20). MG172 family members contain RT and maturase domains, and were predicted to have a conserved Y[I/L]DD active site motif. The motif YIDD was recently reported to display increased efficiency with shorter DNA primers in one G2L4 reference (BioRxiv 10.1101/2022.03.14.484287). MG172 enzymes have an average length of 425 aa and share 32% AAI, which highlights the novelty of these systems.

### Example 12—LTR Retrotransposons (MG151 Family)

### LTR Retrotransposon Bioinformatic Analysis

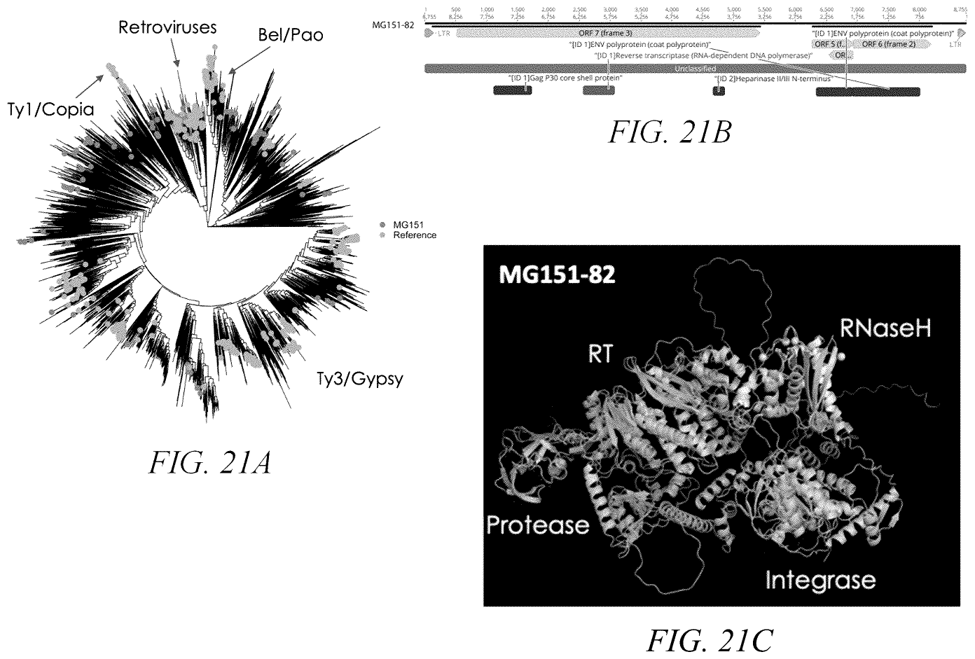

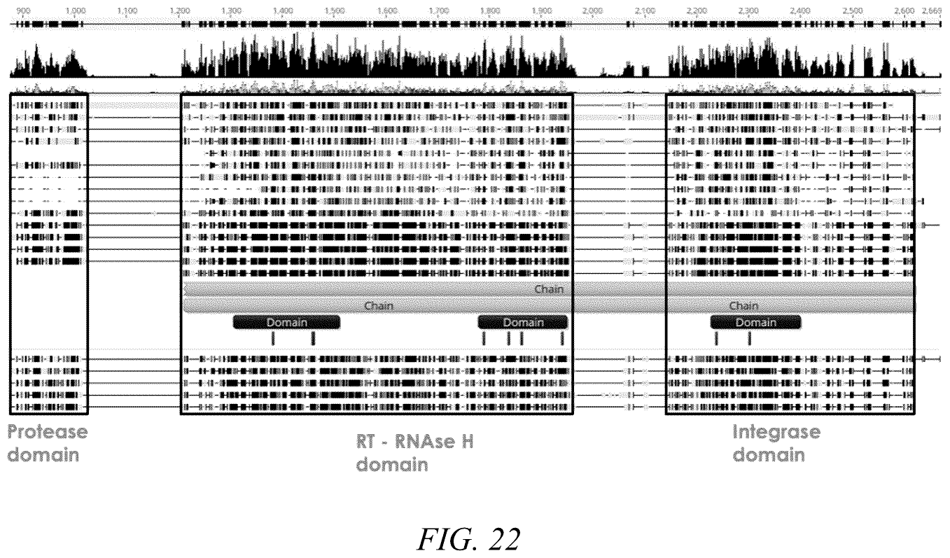

Long terminal repeat (LTR) retrotransposons integrate into their target sites via reverse transcription of an RNA template. The MG151 family of LTR retrotransposons, which include retroviral and non-viral transposons, was identified in the phylogenetic tree in FIG. 4. Full-length proteins containing LTR RT domains were aligned with MAFFT with parameters—globalpair—large. A phylogenetic tree was inferred from this alignment (FIG. 21A). More than 100 non-viral and retroviral RT enzymes of the MG151 family contain RT and RnaseH domains, and are predicted to be active based on the presence of catalytic residues. The LTR RT polyprotein also encodes protease and integrase domains in a similar architecture seen for HIV and MMLV LTR RTs (FIGS. 21A, 21B, 21C, and 22). The RT and other genes, such as gag or envelope, are flanked by long imperfect long terminal repeats (FIG. 21B). MG151 family members are diverse and novel, sharing 30% amino acid identity (FIG. 22).

The polyprotein of LTR retrotransposons is naturally processed into protease, RT and Rnase H, and integrase functional units. Therefore, the MG151 RT-RNAse H functional unit boundaries were determined by a combination of sequence and structural alignments. The 3D structure for MG151 polyproteins was predicted using Alphafold2 (Nature 2021; 596: 583-589; and Nucleic Acids Res 2022; 50: D439-D444) and visualized with PyMOL (https://github.com/schrodinger/pymol-open-source). For example, for MG151-82 (SEQ ID NO: 457), the predicted 3D structure identified discrete protease, RT, RNAseH, and integrase domains separated by unstructured linker regions (FIG. 21C). Therefore, the RT-RNAse H functional unit was determined as the two relevant structural domains flanked by unstructured loops. Trimmed variants containing RT and RNAse H domains were nominated for synthesis and laboratory characterization.

### Testing the In Vitro Activity of LTR Retrotransposon RTs

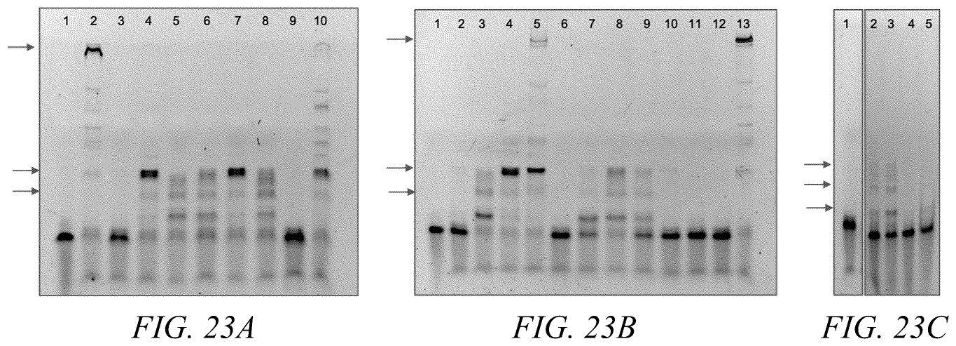

The in vitro activity of LTR retrotransposon RTs (MG151) was assessed by a primer extension reaction containing RT enzyme derived from a cell-free expression system and RNA template annealed to a 5′-FAM labeled primer as described above, in reaction buffer containing 50 mM Tris-HCl pH 8, 75 mM KCl, 3 mM MgCl₂, 1 mM TCEP, and 0.5 mM dNTPs. The resulting cDNA product(s) were separated on a denaturing polyacrylamide gel and visualized using a ChemiDoc on the Gel Green setting. Based on these results, MG151-80 through MG151-84 (FIG. 23A), as well as MG151-87 through MG151-90 (SEQ ID NOs: 524-527), and MG151-92 through MG151-95 (SEQ ID NOs: 529-532) (FIG. 23B) can synthesize cDNA in vitro.

To determine assay conditions under which in vitro activity is observed for Ty3, a control LTR retrotransposon RT, the following four reaction buffers were tested: Buffer A (40 mM Tris-HCl pH 7.5, 0.2 M NaCl, 10 mM MgCl₂, 1 mM TCEP); Buffer B (20 mM Tris pH 7.5, 150 mM KCl, 5 mM MgCl₂, 1 mM TCEP, 2% PEG-8000); Buffer C (10 mm Tris-HCl pH 7.5, 80 mm NaCl, 9 mm MgCl₂, 1 mM TCEP, 0.01% (v/v) Triton X-100); and Buffer D (10 mM Tris pH 7.5, 130 mM NaCl, 9 mM MgCl₂, 1 mM TCEP, 10% glycerol). In vitro activity was observed for Buffers A and B (FIG. 23C).

### Testing Priming Parameters and Processivity on a Structure RNA Template

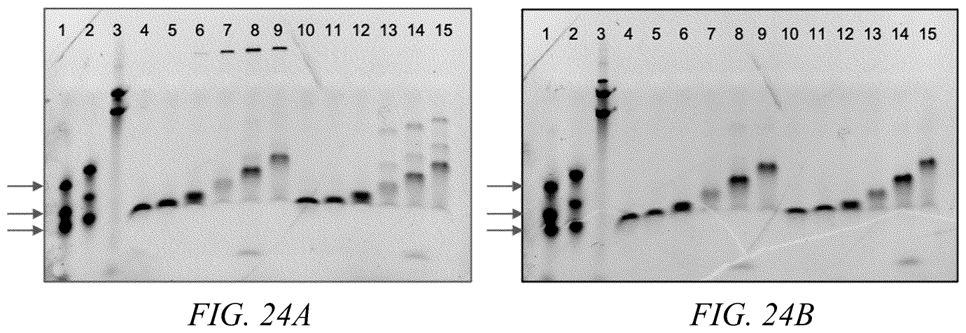

To determine the reverse transcriptase activity of these LTR RTs on a structured RNA template, different primers of length 6, 8, 10, 13, 16, and 20 nt were annealed onto a structured RNA scaffold. These annealed RNA/DNA hybrids were used in a cDNA generation assay equivalent to those used for overall activity. As shown in FIG. 24A and FIG. 24B, MMLV is active on a structured RNA with a primer binding site from 10-20 nt and extends the template completely to the 5′ end, opening up all structure in the template. MG151-89 (SEQ ID NO: 526) is active with primer lengths of 13-20 and can extend approximately 18 nt, the length of pegRNA until the sgRNA scaffold hairpin is reached. MG151-92 (SEQ ID NO: 529) and MG151-97 (SEQ ID NO: 534) were not active on this template at our level of detection.

### Example 13—Retron RTs (MG154, MG155, MG156, MG157, MG158, MG159, and MG160 Families)

### Retron Bioinformatic Analysis

Bacterial retrons are DNA elements of approximately 2000 bp in length that encode an RT-coding gene (ret) and a contiguous non-coding RNA containing inverted sequences, the msr and msd. Retrons employ a unique mechanism for RT-DNA synthesis, in which the ncRNA template folds into a conserved secondary structure, insulated between two inverted repeats (a1/a2). The retron RT recognizes the folded ncRNA, and reverse transcription is initiated from a conserved guanosine 2′OH adjacent to the inverted repeats, forming a 2′-5′ linkage between the template RNA and the nascent cDNA strand. In some retrons this 2′-5′ linkage persists into the mature form of processed RT-DNA, while in others an exonuclease cleaves the DNA product resulting in a free 5′ end. Moreover, the RT targets the msr-msd derived from the same retron as its RNA template, providing specificity that may avoid off-target reverse transcription.

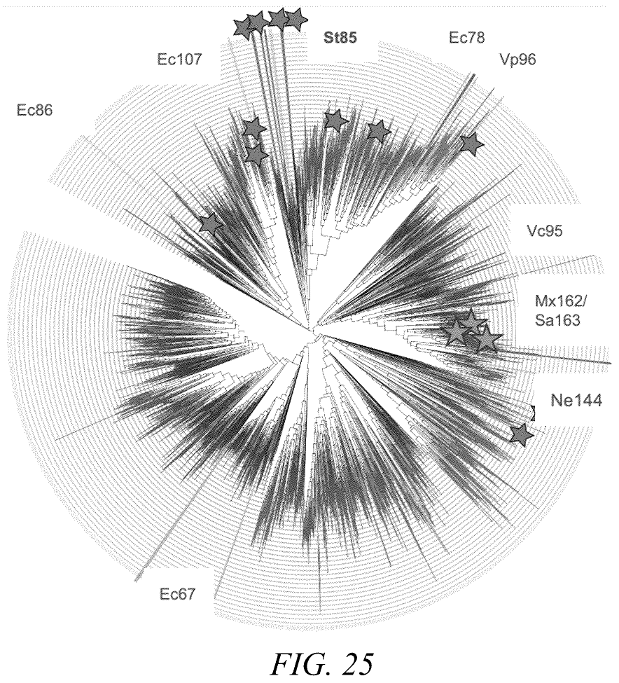

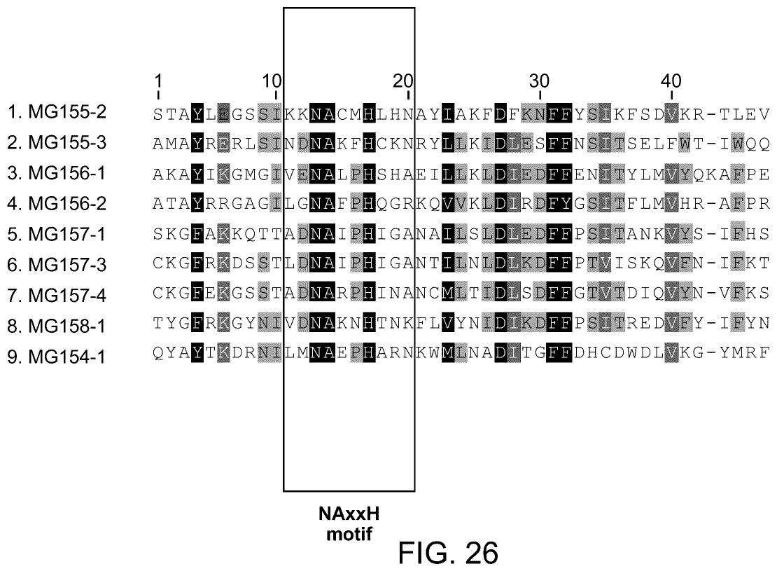

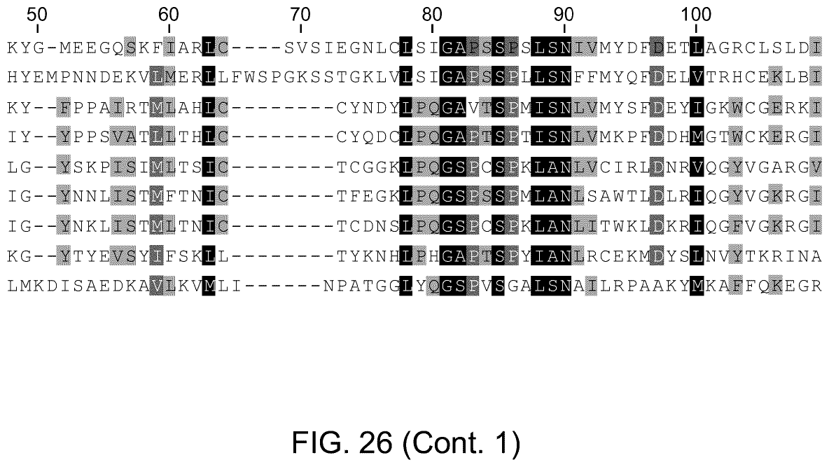

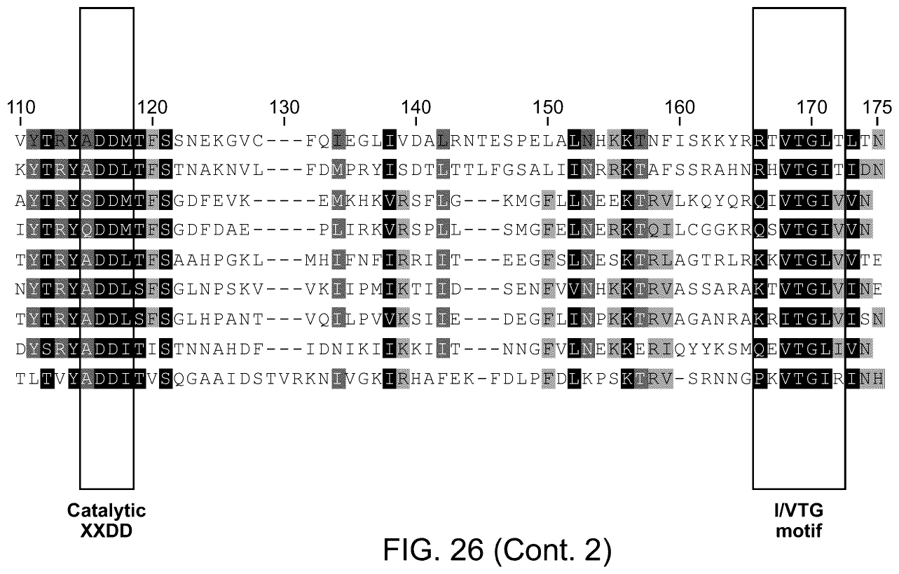

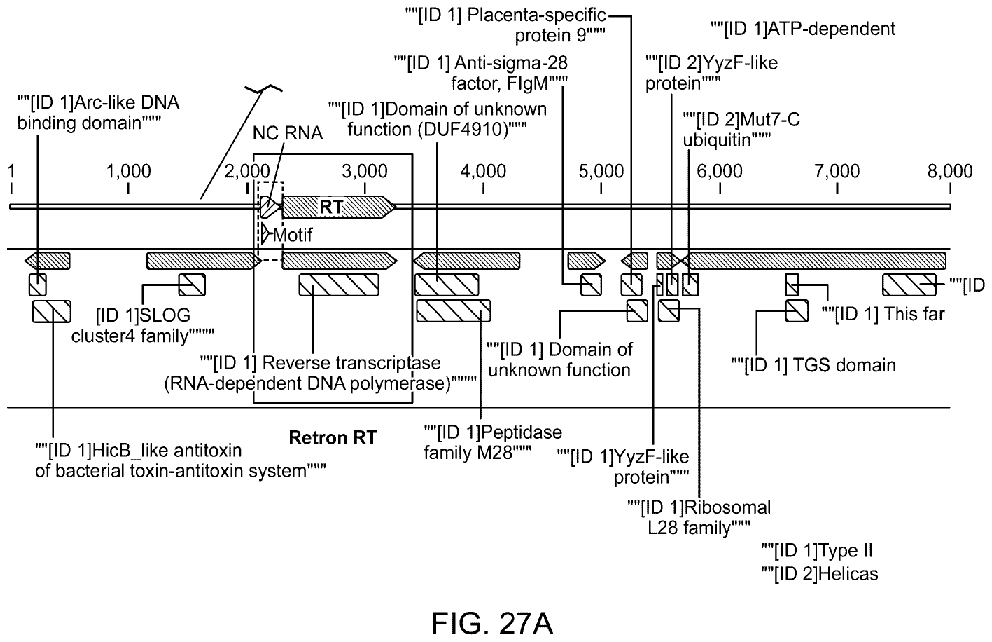

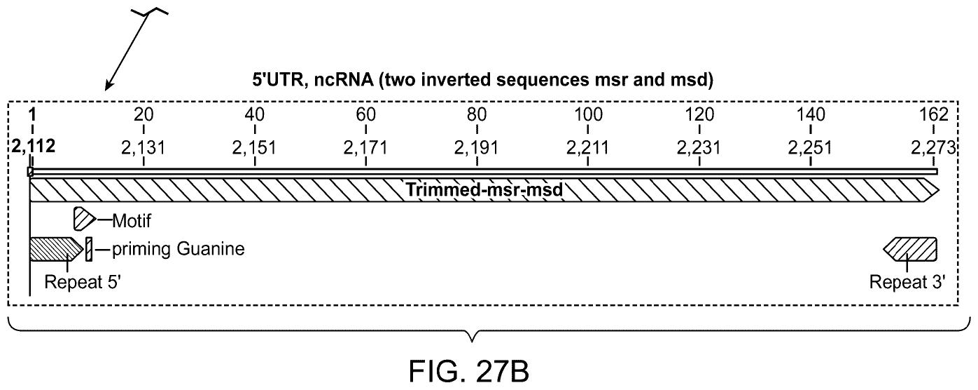

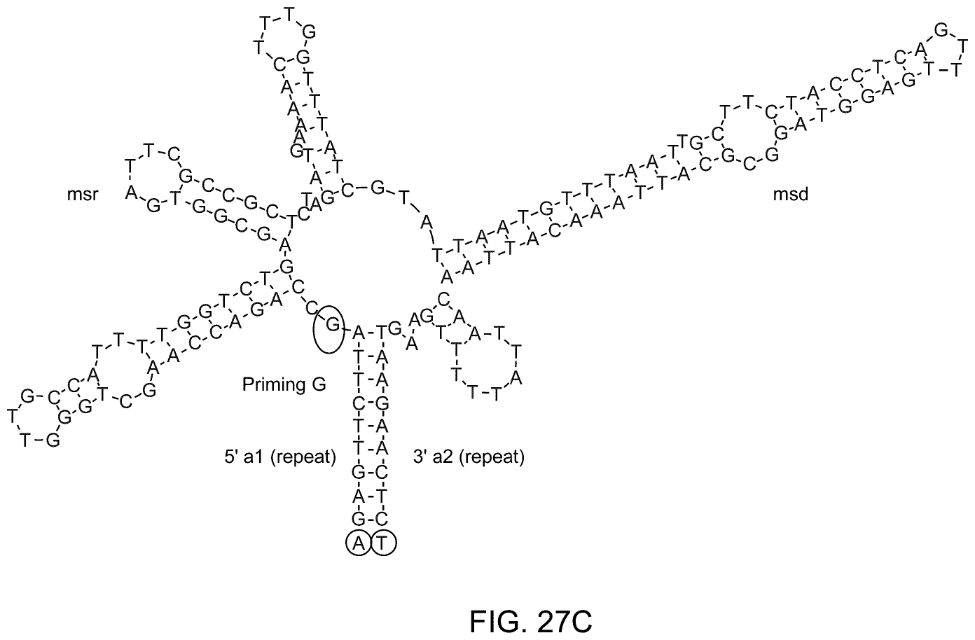

Over 4031 RT domain sequences were identified as retron RTs in the phylogenetic tree in FIG. 4. A subset of 2407 full-length retron protein sequences were selected for further analysis based on the presence of catalytic residues (xxDD) and conserved motifs documented in retron RTs (NaxxH and VTG) (FIGS. 25 and 26). Retrons of families MG154-MG159 and MG173 include members that range between 300 and 650 aa in length, and their 5′ UTR contains predicted ncRNA (msr-msd) trimmed flanked by inverted repeats (FIG. 27).

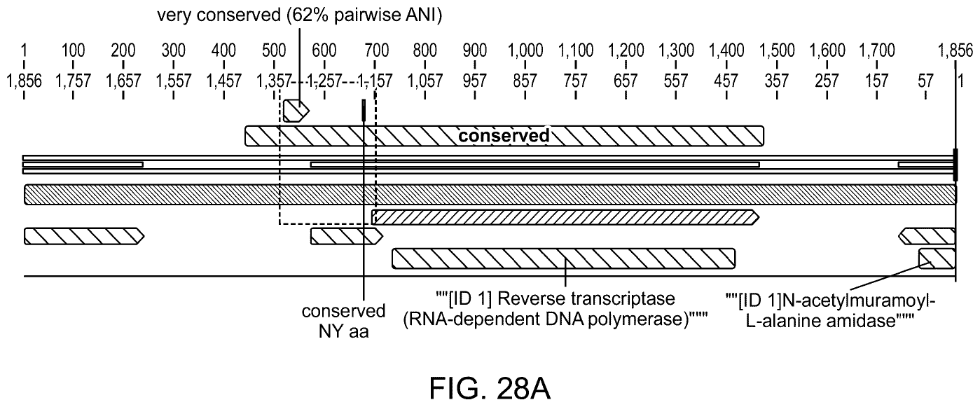

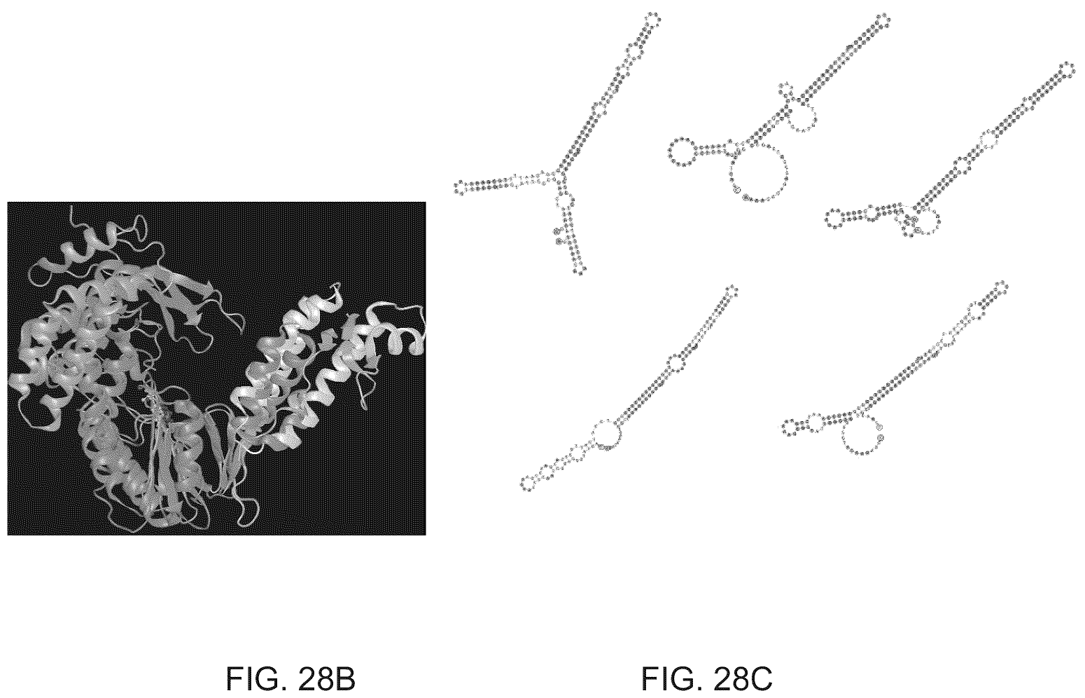

In addition, a divergent group of “retron-like” single-domain RT sequences were identified within the retron clade in FIG. 4. The single-domain RTs of the MG160 family range between 250 and 300 aa and are predicted to be active based on the presence of expected RT catalytic residues [F/Y]XDD. Although there is a lack of retron RT crystal and cryo-EM structures in public databases, 3D structure prediction of MG160-3 (SEQ ID NO: 629) indicates a conserved RT domain that aligns with a Group II intron RT domain (FIGS. 28A and 28B). The 5′ UTR of the MG160 family are conserved among family members and fold into conserved secondary structures (FIG. 28C) that are likely important for element activity or mobilization.

### In Vitro Activity of MG154, MG155, MG156, MG157, MG158, and MG159 Family of Retron-Like RTs

The in vitro activity of retron RTs on a general RNA template was assessed by a primer extension reaction containing RT enzyme derived from a cell-free expression system (PURExpress, NEB). Expression constructs were codon-optimized for E. coli and contained an N-terminal single Strep tag. The substrate for the reaction was 100 nM of RNA template (202 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH (NEB), followed by the addition of 2×RNA loading dye (NEB). The resulting cDNA product(s) were separated on a 10% denaturing polyacrylamide gel and were visualized using a ChemiDoc on the Gel Green setting. Based on these results, the following retron RTs are capable of performing primer extension on a general RNA template that is not their own ncRNA: MG155-2 (SEQ ID NO: 612), MG155-3 (SEQ ID NO: 613), MG156-2 (SEQ ID NO: 617), MG157-5 (SEQ ID NO: 622), and MG159-1 (SEQ ID NO: 624).

In Vitro Activity of MG160 family of Retron-Like RTs

The in vitro activity of retron-like RTs (MG160 family) was assessed by a primer extension reaction containing RT enzyme derived from a cell-free expression system (PURExpress, NEB). Expression constructs were codon-optimized for E. coli and contained an N-terminal single Strep tag. The substrate for the reaction was 100 nM of RNA template (200 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH (NEB), followed by the addition of 2×RNA loading dye (NEB). The resulting cDNA product(s) were separated on a 10% denaturing polyacrylamide gel and were visualized using a ChemiDoc on the Gel Green setting. RT activity was also assessed by qPCR with primers that amplify the full-length cDNA product. Products from the primer extension assay were diluted to ensure cDNA concentrations were within the linear range of detection. The amount of cDNA was quantified by extrapolating values from a standard curve generated with the DNA template of documented concentrations.

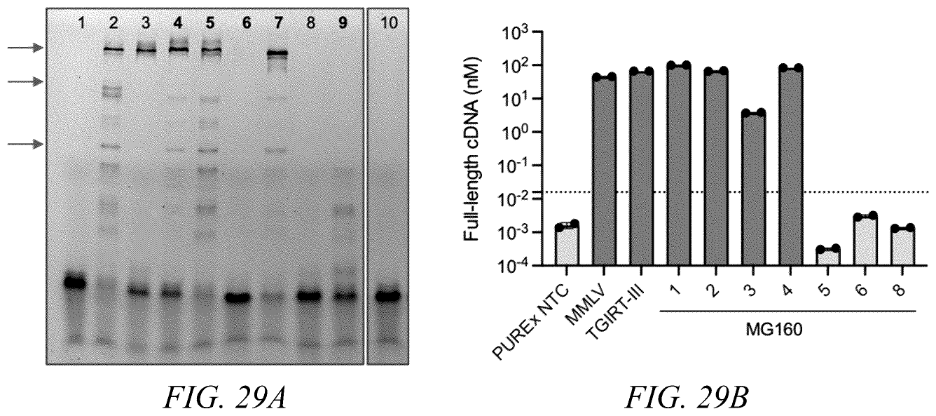

By gel analysis, MG160-1 through MG160-4 (SEQ ID NOs: 627-630) and MG160-6 (SEQ ID NO: 633) are active and had diminished processivity compared to GsI-IIC, a control GII intron Class C RT (FIG. 29A and FIG. 29B). Processivity appears more similar to that of MMLV, a retroviral control RT that produces a similar drop-off pattern of cDNA products (FIG. 29A). By qPCR, MG160-1 through MG160-4 (SEQ ID NOs: 627-630) can produce full-length cDNA, while MG160-6 (SEQ ID NO: 633) produced a less than full-length product (FIG. 29B).

Cell-Free Expression of Retron RTs (MG154, MG155, MG156, MG157, MG158, MG159, and MG173 families) and In Vitro Transcription of Retron ncRNAs

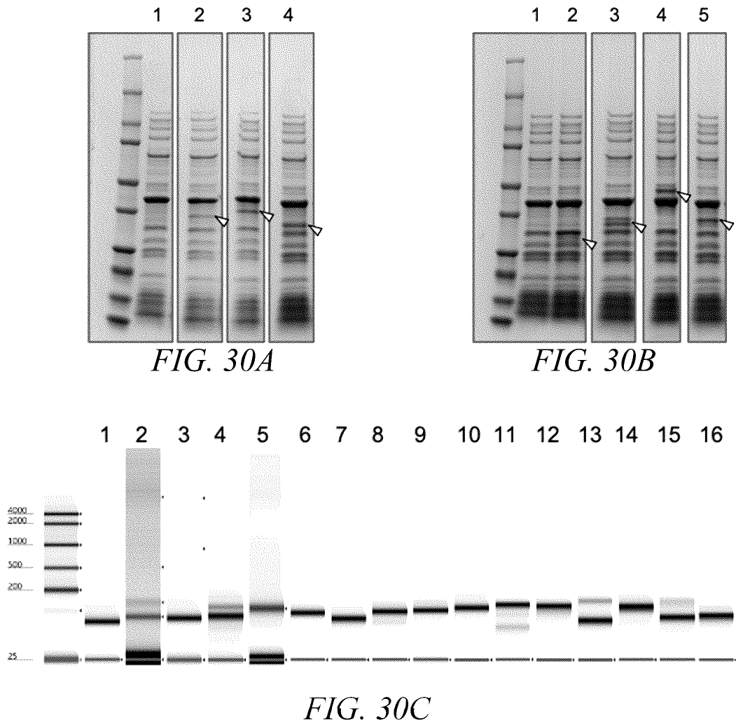

Retron RTs were produced in a cell-free expression system (PURExpress) by incubating 10 ng/μL of a DNA template encoding the E. coli-optimized gene with an N-terminal single Strep tag with the PURExpress components for 2 h at 37° C. All tested retron RTs (MG156-1 (SEQ ID NO: 616), MG156-2 (SEQ ID NO: 617), MG157-1 (SEQ ID NO: 618), MG157-2 (SEQ ID NO: 619), MG157-5 (SEQ ID NO: 622), MG159-1 (SEQ ID NO: 624)) were produced as indicated by SDS-PAGE analysis (FIGS. 30A and 30B).

The retron ncRNAs were generated using the HiScribe T7 in vitro transcription kit (NEB) and a DNA template encoding the respective ncRNA gene following a T7 promoter. The reaction is then incubated with Dnase-I to eliminate the DNA template and then purified by an RNA cleanup kit (Monarch). Quantity of the ncRNA was determined by nanodrop, and the purity was assessed by Tape Station RNA analysis (FIG. 30C).

### Example 14—Testing Retron RT In Vitro Activity (Prophetic)

The retron RT enzyme is produced in a cell-free expression system using a construct containing an E. coli codon-optimized gene with an N-terminal single Strep tag as described above. Expression of the enzyme is confirmed by SDS-PAGE analysis. Retron RT activity on a general template is determined by primer extension assay as described above, containing a 200 nt RNA annealed to a 5′-FAM labeled DNA primer. The resulting cDNA product(s) are detected on a denaturing polyacrylamide gel or by qPCR with primers specific for the full-length cDNA product.

Retron RT in vitro activity on its own ncRNA is assessed in a reaction containing buffer, dNTPs, the retron RT produced from a cell-free expression system, and the refolded ncRNA. RT activity before and after purification of the RT from the cell-free expression system via the N-terminal single Strep tag is compared. After incubation, half of the reaction is treated with Rnase A/T1. Products before and after Rnase A/T1 treatment are evaluated on a denaturing polyacrylamide gel and visualized by SYBR gold staining. In this procedure, Rnase A/T1 is understood to digest away the RNA template and result in a mass shift towards a smaller product containing the ssDNA. Since Rnase H is expected to improve homogeneity of the 5′ and 3′ ssDNA boundaries, the impact of Rnase H on the distribution of products is also evaluated by gel analysis. The covalent linkage between the ncRNA template and ssDNA is confirmed by incubating the RT product with a 5′ to 3′ ssDNA exonuclease (RecJ) before or after treatment with a debranching enzyme (DBR1). RecJ is expected to be able to degrade the ssDNA after DBR1 has removed the 2′-5′ phosphodiester linkage between the RNA and ssDNA.

### Example 15—Determining Retron Msr-Msd Boundaries by NGS (Prophetic)

The msr-msd boundaries are determined by unbiased ligation of adapter sequences to the 5′ and 3′ end of the msDNA product after removal of the 2′-5′ phosphodiester linkage by DBR1. The resulting ligated product is PCR-amplified, library prepped, and subjected to next generation sequencing. Sequencing reads are aligned to the reference sequence to determine the 5′ and 3′ boundaries of the msd. The impact of the presence of Rnase H in the RT reaction on the homogeneity of 5′ and 3′ msd boundaries is also evaluated.

### Example 16—Systematic Evaluation of Insertion Sequences into the Msd on RT Activity (Prophetic)

Sequences of distinct length, predicted secondary structure, and GC-content are inserted into the msd at select insertion sites informed by the msd boundaries determined by NGS and secondary structure predictions of the ncRNA. The impact of these insertion sequences on RT activity are assessed by gel analysis or NGS as described above.

### Example 17—Testing the In Vitro Activity of Novel RTs (Prophetic)

RT activity is assessed using a primer extension assay containing the RT derived from a cell-free expression system and an RNA template annealed to a DNA primer as described above. The resulting cDNA product(s) are detected by a denaturing polyacrylamide gel and qPCR as described above. Detection of cDNA drop-off products on the denaturing gel provides a relative assessment of processivity for novel candidates.

### Example 18—Evaluating the Priming Parameters of Novel RTs (Prophetic)

Optimal primer length is determined by testing the RT's activity on an RNA template annealed to 5′-FAM labeled DNA primers of either 6, 8, 10, 13, 16, or 20 nucleotides in length. The RT is derived from a cell-free expression system as described above. After incubating the reaction, the reaction is quenched via the addition of Rnase H. The size distribution of cDNA products is analyzed on a denaturing polyacrylamide gel as described above. Optimal primer length is determined as the length that enables the RT to convert the most primer into cDNA product. The experimentally determined optimal primer length is then used in subsequent experiments, such as fidelity and processivity assays, to further characterize the RT in vitro.

### Example 19—Evaluating RT Fidelity (Prophetic)

To account for errors introduced during PCR and sequencing, RT fidelity is assessed by a primer extension assay as described above with the exception that a 14-nt unique molecular identifier (UMI) barcode is included in the primer for the reverse transcription reaction. The resulting full-length cDNA product is PCR-amplified, library-prepped, and subjected to next-generation sequencing. Barcodes with >5 reads are analyzed. After aligning to the reference sequence, mutations, insertions, and deletions are counted if the error is present in all sequence reads with the same barcode. Errors present in one but not all sequencing reads are considered to be introduced during PCR or sequencing. Further analysis of substitution, insertion, and deletion profile is performed, in addition to identification of mutation hotspots within the RNA template. The fidelity measurements are also performed with modified bases, e.g. pseudouridine, in the template.

### Example 20—Determining the Processivity Coefficient of RTs (Prophetic)

RT processivity is evaluated using a primer extension assay containing the RT enzyme derived from a cell-free expression system as described above and RNA templates between 1.6 kb-6.6 kb in length annealed to either a 5′-FAM labeled primer (for gel analysis) or unlabeled primer (for sequencing analysis).

Reverse transcription reactions are performed under single cycle conditions to disfavor rebinding of RT enzymes that have dropped off the RNA template during cDNA synthesis. The optimal trap molecule and concentration to achieve single cycle conditions are experimentally determined. The selected conditions are designed to provide sufficient inhibition of cDNA synthesis if incubated before reaction initiation but otherwise are designed to not impact the velocity of the reaction. Optimal trap molecules to test include unrelated RNA templates and unrelated RNA templates annealed to DNA primers of various lengths.

Once single cycle reaction conditions have been optimized, processivity is evaluated by initiating the reaction with the addition of dNTPs and the selected trap molecule after pre-equilibrating the RT with the RNA template annealed to a DNA primer in the reaction buffer. After incubating the reaction, the reaction is quenched by the addition of RnaseH. The size distribution of cDNA products is analyzed on a denaturing polyacrylamide gel as described above or subjected to PCR and library prepped for long-read sequencing. From these experiments, a processivity coefficient is quantified as the template length which yields 50% of the full-length cDNA product. The median length of the cDNA product from the single cycle primer extension reaction is used to estimate the probability that the RT will dissociate on the tested template. From this, the probability that the RT will dissociate at each nucleotide position is calculated, assuming that each dissociation is an independent event and that the probability of dissociation is equal at all nucleotide positions. The processivity coefficient representing the length of template at 50% of RT dissociated is then determined as 1/(2*P_{d}), where P_{d} is the probability of dissociation at each nucleotide.

### Example 21—Systematic Analysis of Challenge Structures on Primer Extension (Prophetic)

To evaluate the impact of challenging templates on RT activity, a primer extension reaction is conducted as stated above, with modifications. The RNA template contains one of the following challenge motifs at fixed distance (100-300 nt) downstream of the primer binding site: homopolymeric stretches, thermodynamically stable GC-rich stem loop, pseudoknot, tRNA, GII intron, and RNA template containing base or backbone modifications (e.g. pseudouridine, phosphothiorate bonds). After quenching the reaction, the size distribution of cDNA products is analyzed by denaturing polyacrylamide gel. An adapter sequence is also unbiasedly ligated to the 3′ ends of the cDNA products using T4 ligase. The ligated product(s) are then PCR-amplified and library prepped for next generation sequencing to identify both sites of RT misincorporation/insertions/deletions and sites of RT drop-off with single nucleotide resolution. Extent of RT drop-off at a given position is quantified by comparing the number of sequencing reads corresponding to the drop-off product to the number of sequencing reads corresponding to the full-length product.

### Example 22—Evaluating Non-Templated Base Additions (Prophetic)

Non-templated addition of bases to the 5′ end of the cDNA product is evaluated by next generation sequencing. Primer extension reactions containing the RT derived from the cell-free expression system and RNA template are conducted as described above. Systematic analysis of different RNA template lengths and sequence motifs at the 5′ end are tested. An adapter sequence is unbiasedly ligated to the 3′ ends of the resulting cDNA products by T4 ligase, resulting in capture of all cDNA products despite the potential heterogeneous nature of their 3′ ends. The ligated product(s) are then PCR-amplified and library prepped for next generation sequencing. Comparison of the expected full-length cDNA reference sequence to experimentally produced cDNA sequences that are longer than full-length enable identification of both the type and number of base additions to the 5′-end that were not templated by the RNA.

### Example 23—Determining 5′ and 3′ UTR Parameters for Activity and Processivity for R2, Non-LTR, and Similar Systems (Prophetic)

Proteins of interest are purified via a Twin-strep tag after IPTG-induced overexpression in E. coli. Purified proteins are tested against 1 kb and 4 kb cargos flanked by the 3′ UTRs identified from their native contexts and the 5′ UTRs plus 400 bp past the start codon. The 5′ and 3′ flanking sequences' effect on activity is assayed via qPCR to sections near the end of the template to determine if cargos with these native features produce superior results.

### Example 24—RT cDNA Synthesis Activity can be Harnessed for Multiple Applications (Prophetic)

Processes dependent on RNA are important in biology, such as expression, processing, modifications, and half-life. Quality control procedures in biotechnology performed on RNA utilize conversion of RNA to cDNA. Therefore, multiple RTs have been used for the production of cDNA libraries over the years. Commercially available RTs used for these purposes include the MMLV RT, AMV RT, and GsI-IIC RT (TGIRT). The first two represent retroviral RTs, while the latter is a GII intron derived RT. GII intron derived RTs, as well as non-LTR derived RTs, show several advantages compared to their retroviral counterparts. For example, they are more processive, reading through structural and modified RNAs. Structural or modified RNAs may not be optimal substrates for retroviral RTs, as they create early termination products that can be misinterpreted as RNA fragments. In addition, the ability to template switch of some RTs can be harnessed for early adaptor addition, making the adaptor ligation procedures less important during library preparation. Therefore, highly processive RTs are suitable for the generation of libraries with complex RNA. Further, some highly processive RTs are generally smaller than currently used retroviral RTs, making their production and associated downstream processes easier. Several novel RTs described herein outperform the commercially available TGIRT enzyme, some with over 10-fold its cDNA synthesis activity. As such, many of these novel RTs show great promise for their commercial application for cDNA synthesis kits.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

TABLE 3
Protein and nucleic acid sequences referred to herein

| | Sequence | SEQ ID | | |
|---|---|---|---|---|
| Cat. | Number | NO: | Description | Type |
| MG140 transposition proteins | | 1 | MG140-1-R2 transposition protein | protein |
| MG140 transposition proteins | | 2 | MG140-2-R2 transposition protein | protein |
| MG140 transposition proteins | | 3 | MG140-3-R2 transposition protein | protein |
| MG140 transposition proteins | | 4 | MG140-4-R2 transposition protein | protein |
| MG140 transposition proteins | | 5 | MG140-5-R2 transposition protein | protein |
| MG140 transposition proteins | | 6 | MG140-6-R2 transposition protein | protein |
| MG140 transposition proteins | | 7 | MG140-7-R2 transposition protein | protein |
| MG140 transposition proteins | | 8 | MG140-8-R2 transposition protein | protein |
| MG140 transposition proteins | | 9 | MG140-147 transposition protein | protein |
| MG140 transposition proteins | | 10 | MG140-9-R2 transposition protein | protein |
| MG140 transposition proteins | | 11 | MG140-10-R2 transposition protein | protein |
| MG140 transposition proteins | | 12 | MG140-11-R2 transposition protein | protein |
| MG140 transposition proteins | | 13 | MG140-12-R2 transposition protein | protein |
| MG140 transposition proteins | | 14 | MG140-13-R2 transposition protein | protein |
| MG140 transposition proteins | | 15 | MG140-14-R2 transposition protein | protein |
| MG140 transposition proteins | | 16 | MG140-15-R2 transposition protein | protein |
| MG140 transposition proteins | | 17 | MG140-16-R2 transposition protein | protein |
| MG140 transposition proteins | | 18 | MG140-17-R2 transposition protein | protein |
| MG140 transposition proteins | | 19 | MG140-18-R2 transposition protein | protein |
| MG140 transposition proteins | | 20 | MG140-19-R2 transposition protein | protein |
| MG140 transposition proteins | | 21 | MG140-20-R2 transposition protein | protein |
| MG140 transposition proteins | | 22 | MG140-21-R2 transposition protein | protein |
| MG140 transposition proteins | | 23 | MG140-22-R2 transposition protein | protein |
| MG140 transposition proteins | | 24 | MG140-23-R2 transposition protein | protein |
| MG140 transposition proteins | | 25 | MG140-24-R2 transposition protein | protein |
| MG140 transposition proteins | | 26 | MG140-25-R2 transposition protein | protein |
| MG140 transposition proteins | | 27 | MG140-26-R2 transposition protein | protein |
| MG140 transposition proteins | | 28 | MG140-27-R2 transposition protein | protein |
| MG140 transposition proteins | | 29 | MG140-28-R2 transposition protein | protein |
| MG153 RT MCP fusions | | 30 | FH-MCP-MG153-1 | nucleotide |
| MG153 RT MCP fusions | | 31 | FH-MCP-MG153-2 | nucleotide |
| MG153 RT MCP fusions | | 32 | FH-MCP-MG153-3 | nucleotide |
| Nanoluciferase templates | | 33 | Nanoluciferase Template | nucleotide |
| Complementary DNA primers | | 34 | Complementary DNA primer | nucleotide |
| PCR primers | | 35 | Forward primer for 100/542 bp amplicon | nucleotide |
| PCR primers | | 36 | Reverse primer for 100 bp amplicon | nucleotide |
| PCR primers | | 37 | Reverse primer for 542 bp amplicon | nucleotide |
| PCR amplicons derived from | | 38 | 100 bp amplicon | nucleotide |
| cDNA | | | | |
| PCR amplicons derived from | | 39 | 542 bp amplicon | nucleotide |
| cDNA | | | | |
| MG153 RT MCP fusions | | 40 | FH-MCP-MG153-4 | nucleotide |
| MG153 RT MCP fusions | | 41 | FH-MCP-MG153-7 | nucleotide |
| MG153 RT MCP fusions | | 42 | FH-MCP-MG153-8 | nucleotide |
| MG153 RT MCP fusions | | 43 | FH-MCP-MG153-9 | nucleotide |
| MG153 RT MCP fusions | | 44 | FH-MCP-MG153-10 | nucleotide |
| MG153 RT MCP fusions | | 45 | FH-MCP-MG153-11 | nucleotide |
| MG153 RT MCP fusions | | 46 | FH-MCP-MG153-12 | nucleotide |
| MG153 RT MCP fusions | | 47 | FH-MCP-MG153-13 | nucleotide |
| MG153 RT MCP fusions | | 48 | FH-MCP-MG153-15 | nucleotide |
| MG153 RT MCP fusions | | 49 | FH-MCP-MG153-16 | nucleotide |
| MG153 RT MCP fusions | | 50 | FH-MCP-MG153-21 | nucleotide |
| primer to amplify from | | 51 | LA_061_pmgx_txtl_rv | nucleotide |
| pET21(+) | | | | |
| primer to amplify from | | 52 | LA_062_pmgx_txtl_fw | nucleotide |
| pET21(+) | | | | |
| RT RNA template | | 53 | Structured RT template RNA to emx1 | nucleotide |
| RT RNA template | | 54 | Structured RT template RNA to emx1 | nucleotide |
| | | | (5′ truncated) | |
| RT RNA template | | 55 | Small RT template | nucleotide |
| FAM-labeled oligo for cDNA | | 56 | LA321_282_FAM | nucleotide |
| assay | | | | |
| NGS primer | | 57 | LA065_NGS_enrich_fw | nucleotide |
| NGS primer | | 58 | LA395_282_NGS | nucleotide |
| NGS primer | | 59 | LA396_RT_short_NGS | nucleotide |
| FAM-labeled oligo for cDNA | A60 | | LA423_emx1_6 pbs | nucleotide |
| assay | | | | |
| FAM-labeled oligo for cDNA | A61 | | LA424_emx1_8 pbs | nucleotide |
| assay | | | | |
| FAM-labeled oligo for cDNA | | 62 | LA425_emx1_10 pbs | nucleotide |
| assay | | | | |
| FAM-labeled oligo for cDNA | | 63 | LA426_emx1_13 pbs | nucleotide |
| assay | | | | |
| FAM-labeled oligo for cDNA | | 64 | LA427_emx1_20 pbs | nucleotide |
| assay | | | | |
| UMI primer for NGS fidelity | | 65 | LA430_umi_cdna | nucleotide |
| MG153 Strep tagged genes | | 66 | Nstrep-MG153-1 | nucleotide |
| MG153 Strep tagged genes | | 67 | Nstrep-MG153-2 | nucleotide |
| MG153 Strep tagged genes | | 68 | Nstrep-MG153-3 | nucleotide |
| MG153 Strep tagged genes | | 69 | Nstrep-MG153-4 | nucleotide |
| MG153 Strep tagged genes | | 70 | Nstrep-MG153-5 | nucleotide |
| MG153 Strep tagged genes | | 71 | Nstrep-MG153-6 | nucleotide |
| MG153 Strep tagged genes | | 72 | Nstrep-MG153-7 | nucleotide |
| MG153 Strep tagged genes | | 73 | Nstrep-MG153-8 | nucleotide |
| MG153 Strep tagged genes | | 74 | Nstrep-MG153-9 | nucleotide |
| MG153 Strep tagged genes | | 75 | Nstrep-MG153-10 | nucleotide |
| MG153 Strep tagged genes | | 76 | Nstrep-MG153-11 | nucleotide |
| MG153 Strep tagged genes | | 77 | Nstrep-MG153-12 | nucleotide |
| MG153 Strep tagged genes | | 78 | Nstrep-MG153-13 | nucleotide |
| MG153 Strep tagged genes | | 79 | Nstrep-MG153-14 | nucleotide |
| MG153 Strep tagged genes | | 80 | Nstrep-MG153-15 | nucleotide |
| MG153 Strep tagged genes | | 81 | Nstrep-MG153-16 | nucleotide |
| MG153 Strep tagged genes | | 82 | Nstrep-MG153-17 | nucleotide |
| MG153 Strep tagged genes | | 83 | Nstrep-MG153-18 | nucleotide |
| MG153 Strep tagged genes | | 84 | Nstrep-MG153-19 | nucleotide |
| MG153 Strep tagged genes | | 85 | Nstrep-MG153-20 | nucleotide |
| MG153 Strep tagged genes | | 86 | Nstrep-MG153-21 | nucleotide |
| MG153 Strep tagged genes | | 87 | Nstrep-MG153-25 | nucleotide |
| MG153 Strep tagged genes | | 88 | Nstrep-MG153-26 | nucleotide |
| MG153 Strep tagged genes | | 89 | Nstrep-MG153-27 | nucleotide |
| MG153 Strep tagged genes | | 90 | Nstrep-MG153-28 | nucleotide |
| MG153 Strep tagged genes | | 91 | Nstrep-MG153-29 | nucleotide |
| MG153 Strep tagged genes | | 92 | Nstrep-MG153-30 | nucleotide |
| MG153 Strep tagged genes | | 93 | Nstrep-MG153-31 | nucleotide |
| MG153 Strep tagged genes | | 94 | Nstrep-MG153-32 | nucleotide |
| MG153 Strep tagged genes | | 95 | Nstrep-MG153-33 | nucleotide |
| MG153 Strep tagged genes | | 96 | Nstrep-MG153-34 | nucleotide |
| MG153 Strep tagged genes | | 97 | Nstrep-MG153-35 | nucleotide |
| MG153 Strep tagged genes | | 98 | Nstrep-MG153-36 | nucleotide |
| MG153 Strep tagged genes | | 99 | Nstrep-MG153-37 | nucleotide |
| MG153 Strep tagged genes | | 100 | Nstrep-MG153-38 | nucleotide |
| MG153 Strep tagged genes | | 101 | Nstrep-MG153-39 | nucleotide |
| MG153 Strep tagged genes | | 102 | Nstrep-MG153-40 | nucleotide |
| MG153 Strep tagged genes | | 103 | Nstrep-MG153-41 | nucleotide |
| MG153 Strep tagged genes | | 104 | Nstrep-MG153-42 | nucleotide |
| MG153 Strep tagged genes | | 105 | Nstrep-MG153-43 | nucleotide |
| MG153 Strep tagged genes | | 106 | Nstrep-MG153-44 | nucleotide |
| MG153 Strep tagged genes | | 107 | Nstrep-MG153-45 | nucleotide |
| MG153 Strep tagged genes | | 108 | Nstrep-MG153-46 | nucleotide |
| MG153 Strep tagged genes | | 109 | Nstrep-MG153-47 | nucleotide |
| MG153 Strep tagged genes | | 110 | Nstrep-MG153-48 | nucleotide |
| MG153 Strep tagged genes | | 111 | Nstrep-MG153-49 | nucleotide |
| MG153 Strep tagged genes | | 112 | Nstrep-MG153-50 | nucleotide |
| MG153 Strep tagged genes | | 113 | Nstrep-MG153-51 | nucleotide |
| MG153 Strep tagged genes | | 114 | Nstrep-MG153-52 | nucleotide |
| MG153 Strep tagged genes | | 115 | Nstrep-MG153-53 | nucleotide |
| MG153 Strep tagged genes | | 116 | Nstrep-MG153-54 | nucleotide |
| MG153 Strep tagged genes | | 117 | Nstrep-MG153-55 | nucleotide |
| MG153 Strep tagged genes | | 118 | Nstrep-MG153-56 | nucleotide |
| MG153 Strep tagged genes | | 119 | Nstrep-MG153-57 | nucleotide |
| MG153 E. coli codon | | 120 | MG153-1 | nucleotide |
| optimized genes | | | | |
| MG153 E. coli codon | | 121 | MG153-2 | nucleotide |
| optimized genes | | | | |
| MG153 E. coli codon | | 122 | MG153-3 | nucleotide |
| optimized genes | | | | |
| MG153 E. coli codon | | 123 | MG153-4 | nucleotide |
| optimized genes | | | | |
| MG153 E. coli codon | | 124 | MG153-5 | nucleotide |
| optimized genes | | | | |
| MG153 E. coli codon | | 125 | MG153-6 | nucleotide |
| optimized genes | | | | |
| MG153 E. coli codon | | 126 | MG153-7 | nucleotide |
| optimized genes | | | | |
| MG153 E. coli codon | | 127 | MG153-8 | nucleotide |
| optimized genes | | | | |
| MG153 E. coli codon | | 128 | MG153-9 | nucleotide |
| optimized genes | | | | |
| MG153 E. coli codon | | 129 | MG153-10 | nucleotide |
| optimized genes | | | | |
| MG153 E. coli codon | | 130 | MG153-11 | nucleotide |
| optimized genes | | | | |
| MG153 E. coli codon | | 131 | MG153-12 | nucleotide |
| optimized genes | | | | |
| MG153 E. coli codon | | 132 | MG153-13 | nucleotide |
| optimized genes | | | | |
| MG153 E. coli codon | | 133 | MG153-14 | nucleotide |
| optimized genes | | | | |
| MG153 E. coli codon | | 134 | MG153-15 | nucleotide |
| optimized genes | | | | |
| MG153 E. coli codon | | 135 | MG153-16 | nucleotide |
| optimized genes | | | | |
| MG153 E. coli codon | | 136 | MG153-17 | nucleotide |
| optimized genes | | | | |
| MG153 E. coli codon | | 137 | MG153-18 | nucleotide |
| optimized genes | | | | |
| MG153 E. coli codon | | 138 | MG153-19 | nucleotide |
| optimized genes | | | | |
| MG153 E. coli codon | | 139 | MG153-20 | nucleotide |
| optimized genes | | | | |
| MG153 E. coli codon | | 140 | MG153-21 | nucleotide |
| optimized genes | | | | |
| MG153 E. coli codon | | 141 | MG153-25 | nucleotide |
| optimized genes | | | | |
| MG153 E. coli codon | | 142 | MG153-26 | nucleotide |
| optimized genes | | | | |
| MG153 E. coli codon | | 143 | MG153-27 | nucleotide |
| optimized genes | | | | |
| MG153 E. coli codon | | 144 | MG153-28 | nucleotide |
| optimized genes | | | | |
| MG153 E. coli codon | | 145 | MG153-29 | nucleotide |
| optimized genes | | | | |
| MG153 E. coli codon | | 146 | MG153-30 | nucleotide |
| optimized genes | | | | |
| MG153 E. coli codon | | 147 | MG153-31 | nucleotide |
| optimized genes | | | | |
| MG153 E. coli codon | | 148 | MG153-32 | nucleotide |
| optimized genes | | | | |
| MG153 E. coli codon | | 149 | MG153-33 | nucleotide |
| optimized genes | | | | |
| MG153 E. coli codon | | 150 | MG153-34 | nucleotide |
| optimized genes | | | | |
| MG153 E. coli codon | | 151 | MG153-35 | nucleotide |
| optimized genes | | | | |
| MG153 E. coli codon | | 152 | MG153-36 | nucleotide |
| optimized genes | | | | |
| MG153 E. coli codon | | 153 | MG153-37 | nucleotide |
| optimized genes | | | | |
| MG153 E. coli codon | | 154 | MG153-38 | nucleotide |
| optimized genes | | | | |
| MG153 E. coli codon | | 155 | MG153-39 | nucleotide |
| optimized genes | | | | |
| MG153 E. coli codon | | 156 | MG153-40 | nucleotide |
| optimized genes | | | | |
| MG153 E. coli codon | | 157 | MG153-41 | nucleotide |
| optimized genes | | | | |
| MG153 E. coli codon | | 158 | MG153-42 | nucleotide |
| optimized genes | | | | |
| MG153 E. coli codon | | 159 | MG153-43 | nucleotide |
| optimized genes | | | | |
| MG153 E. coli codon | | 160 | MG153-44 | nucleotide |
| optimized genes | | | | |
| MG153 E. coli codon | | 161 | MG153-45 | nucleotide |
| optimized genes | | | | |
| MG153 E. coli codon | | 162 | MG153-46 | nucleotide |
| optimized genes | | | | |
| MG153 E. coli codon | | 163 | MG153-47 | nucleotide |
| optimized genes | | | | |
| MG153 E. coli codon | | 164 | MG153-48 | nucleotide |
| optimized genes | | | | |
| MG153 E. coli codon | | 165 | MG153-49 | nucleotide |
| optimized genes | | | | |
| MG153 E. coli codon | | 166 | MG153-50 | nucleotide |
| optimized genes | | | | |
| MG153 E. coli codon | | 167 | MG153-51 | nucleotide |
| optimized genes | | | | |
| MG153 E. coli codon | | 168 | MG153-52 | nucleotide |
| optimized genes | | | | |
| MG153 E. coli codon | | 169 | MG153-53 | nucleotide |
| optimized genes | | | | |
| MG153 E. coli codon | | 170 | MG153-54 | nucleotide |
| optimized genes | | | | |
| MG153 E. coli codon | | 171 | MG153-55 | nucleotide |
| optimized genes | | | | |
| MG153 E. coli codon | | 172 | MG153-56 | nucleotide |
| optimized genes | | | | |
| MG153 E. coli codon | | 173 | MG153-57 | nucleotide |
| optimized genes | | | | |
| MG160 Strep tagged genes | | 174 | Nstrep-MG160-1 | nucleotide |
| MG160 Strep tagged genes | | 175 | Nstrep-MG160-2 | nucleotide |
| MG160 Strep tagged genes | | 176 | Nstrep-MG160-3 | nucleotide |
| MG160 Strep tagged genes | | 177 | Nstrep-MG160-4 | nucleotide |
| MG160 Strep tagged genes | | 178 | Nstrep-MG160-5 | nucleotide |
| MG160 Strep tagged genes | | 179 | Nstrep-MG160-6 | nucleotide |
| MG160 Strep tagged genes | | 180 | Nstrep-MG160-8 | nucleotide |
| MG160 E. coli codon | | 181 | MG160-1 | nucleotide |
| optimized genes | | | | |
| MG160 E. coli codon | | 182 | MG160-2 | nucleotide |
| optimized genes | | | | |
| MG160 E. coli codon | | 183 | MG160-3 | nucleotide |
| optimized genes | | | | |
| MG160 E. coli codon | | 184 | MG160-4 | nucleotide |
| optimized genes | | | | |
| MG160 E. coli codon | | 185 | MG160-5 | nucleotide |
| optimized genes | | | | |
| MG160 E. coli codon | | 186 | MG160-6 | nucleotide |
| optimized genes | | | | |
| MG160 E. coli codon | | 187 | MG160-8 | nucleotide |
| optimized genes | | | | |
| MG163 Strep tagged genes | | 188 | Nstrep-MG163-1 | nucleotide |
| MG163 Strep tagged genes | | 189 | Nstrep-MG163-2 | nucleotide |
| MG163 Strep tagged genes | | 190 | Nstrep-MG163-3 | nucleotide |
| MG163 Strep tagged genes | | 191 | Nstrep-MG163-4 | nucleotide |
| MG163 Strep tagged genes | | 192 | Nstrep-MG163-5 | nucleotide |
| MG163 E. coli codon | | 193 | MG163-1 | nucleotide |
| optimized genes | | | | |
| MG163 E. coli codon | | 194 | MG163-2 | nucleotide |
| optimized genes | | | | |
| MG163 E. coli codon | | 195 | MG163-3 | nucleotide |
| optimized genes | | | | |
| MG163 E. coli codon | | 196 | MG163-4 | nucleotide |
| optimized genes | | | | |
| MG163 E. coli codon | | 197 | MG163-5 | nucleotide |
| optimized genes | | | | |
| MG164 Strep tagged genes | | 198 | Nstrep-MG164-1 | nucleotide |
| MG164 Strep tagged genes | | 199 | Nstrep-MG164-2 | nucleotide |
| MG164 Strep tagged genes | | 200 | Nstrep-MG164-3 | nucleotide |
| MG164 Strep tagged genes | | 201 | Nstrep-MG164-4 | nucleotide |
| MG164 Strep tagged genes | | 202 | Nstrep-MG164-5 | nucleotide |
| MG164 E. coli codon | | 203 | MG164-1 | nucleotide |
| optimized genes | | | | |
| MG164 E. coli codon | | 204 | MG164-2 | nucleotide |
| optimized genes | | | | |
| MG164 E. coli codon | | 205 | MG164-3 | nucleotide |
| optimized genes | | | | |
| MG164 E. coli codon | | 206 | MG164-4 | nucleotide |
| optimized genes | | | | |
| MG164 E. coli codon | | 207 | MG164-5 | nucleotide |
| optimized genes | | | | |
| MG165 Strep tagged genes | | 208 | Nstrep-MG165-1 | nucleotide |
| MG165 Strep tagged genes | | 209 | Nstrep-MG165-2 | nucleotide |
| MG165 Strep tagged genes | | 210 | Nstrep-MG165-3 | nucleotide |
| MG165 Strep tagged genes | | 211 | Nstrep-MG165-4 | nucleotide |
| MG165 Strep tagged genes | | 212 | Nstrep-MG165-5 | nucleotide |
| MG165 Strep tagged genes | | 213 | Nstrep-MG165-6 | nucleotide |
| MG165 Strep tagged genes | | 214 | Nstrep-MG165-7 | nucleotide |
| MG165 Strep tagged genes | | 215 | Nstrep-MG165-8 | nucleotide |
| MG165 Strep tagged genes | | 216 | Nstrep-MG165-9 | nucleotide |
| MG165 E. coli codon | | 217 | MG165-1 | nucleotide |
| optimized genes | | | | |
| MG165 E. coli codon | | 218 | MG165-2 | nucleotide |
| optimized genes | | | | |
| MG165 E. coli codon | | 219 | MG165-3 | nucleotide |
| optimized genes | | | | |
| MG165 E. coli codon | | 220 | MG165-4 | nucleotide |
| optimized genes | | | | |
| MG165 E. coli codon | | 221 | MG165-5 | nucleotide |
| optimized genes | | | | |
| MG165 E. coli codon | | 222 | MG165-6 | nucleotide |
| optimized genes | | | | |
| MG165 E. coli codon | | 223 | MG165-7 | nucleotide |
| optimized genes | | | | |
| MG165 E. coli codon | | 224 | MG165-8 | nucleotide |
| optimized genes | | | | |
| MG165 E. coli codon | | 225 | MG165-9 | nucleotide |
| optimized genes | | | | |
| MG166 Strep tagged genes | | 226 | Nstrep-MG166-1 | nucleotide |
| MG166 Strep tagged genes | | 227 | Nstrep-MG166-2 | nucleotide |
| MG166 Strep tagged genes | | 228 | Nstrep-MG166-3 | nucleotide |
| MG166 Strep tagged genes | | 229 | Nstrep-MG166-4 | nucleotide |
| MG166 Strep tagged genes | | 230 | Nstrep-MG166-5 | nucleotide |
| MG166 E. coli codon | | 231 | MG166-1 | nucleotide |
| optimized genes | | | | |
| MG166 E. coli codon | | 232 | MG166-2 | nucleotide |
| optimized genes | | | | |
| MG166 E. coli codon | | 233 | MG166-3 | nucleotide |
| optimized genes | | | | |
| MG166 E. coli codon | | 234 | MG166-4 | nucleotide |
| optimized genes | | | | |
| MG166 E. coli codon | | 235 | MG166-5 | nucleotide |
| optimized genes | | | | |
| MG167 Strep tagged genes | | 236 | Nstrep-MG167-1 | nucleotide |
| MG167 Strep tagged genes | | 237 | Nstrep-MG167-2 | nucleotide |
| MG167 Strep tagged genes | | 238 | Nstrep-MG167-3 | nucleotide |
| MG167 Strep tagged genes | | 239 | Nstrep-MG167-4 | nucleotide |
| MG167 Strep tagged genes | | 240 | Nstrep-MG167-5 | nucleotide |
| MG167 E. coli codon | | 241 | MG167-1 | nucleotide |
| optimized genes | | | | |
| MG167 E. coli codon | | 242 | MG167-2 | nucleotide |
| optimized genes | | | | |
| MG167 E. coli codon | | 243 | MG167-3 | nucleotide |
| optimized genes | | | | |
| MG167 E. coli codon | | 244 | MG167-4 | nucleotide |
| optimized genes | | | | |
| MG167 E. coli codon | | 245 | MG167-5 | nucleotide |
| optimized genes | | | | |
| MG168 Strep tagged genes | | 246 | Nstrep-MG168-1 | nucleotide |
| MG168 Strep tagged genes | | 247 | Nstrep-MG168-2 | nucleotide |
| MG168 Strep tagged genes | | 248 | Nstrep-MG168-3 | nucleotide |
| MG168 Strep tagged genes | | 249 | Nstrep-MG168-4 | nucleotide |
| MG168 Strep tagged genes | | 250 | Nstrep-MG168-5 | nucleotide |
| MG168 E. coli codon | | 251 | MG168-1 | nucleotide |
| optimized genes | | | | |
| MG168 E. coli codon | | 252 | MG168-2 | nucleotide |
| optimized genes | | | | |
| MG168 E. coli codon | | 253 | MG168-3 | nucleotide |
| optimized genes | | | | |
| MG168 E. coli codon | | 254 | MG168-4 | nucleotide |
| optimized genes | | | | |
| MG168 E. coli codon | | 255 | MG168-5 | nucleotide |
| optimized genes | | | | |
| MG169 Strep tagged genes | | 256 | Nstrep-MG169-1 | nucleotide |
| MG169 Strep tagged genes | | 257 | Nstrep-MG169-2 | nucleotide |
| MG169 Strep tagged genes | | 258 | Nstrep-MG169-3 | nucleotide |
| MG169 Strep tagged genes | | 259 | Nstrep-MG169-4 | nucleotide |
| MG169 Strep tagged genes | | 260 | Nstrep-MG169-5 | nucleotide |
| MG169 Strep tagged genes | | 261 | Nstrep-MG169-6 | nucleotide |
| MG169 Strep tagged genes | | 262 | Nstrep-MG169-7 | nucleotide |
| MG169 Strep tagged genes | | 263 | Nstrep-MG169-8 | nucleotide |
| MG169 Strep tagged genes | | 264 | Nstrep-MG169-9 | nucleotide |
| MG169 Strep tagged genes | | 265 | Nstrep-MG169-10 | nucleotide |
| MG169 Strep tagged genes | | 266 | Nstrep-MG169-11 | nucleotide |
| MG169 E. coli codon | | 267 | MG169-1 | nucleotide |
| optimized genes | | | | |
| MG169 E. coli codon | | 268 | MG169-2 | nucleotide |
| optimized genes | | | | |
| MG169 E. coli codon | | 269 | MG169-3 | nucleotide |
| optimized genes | | | | |
| MG169 E. coli codon | | 270 | MG169-4 | nucleotide |
| optimized genes | | | | |
| MG169 E. coli codon | | 271 | MG169-5 | nucleotide |
| optimized genes | | | | |
| MG169 E. coli codon | | 272 | MG169-6 | nucleotide |
| optimized genes | | | | |
| MG169 E. coli codon | | 273 | MG169-7 | nucleotide |
| optimized genes | | | | |
| MG169 E. coli codon | | 274 | MG169-8 | nucleotide |
| optimized genes | | | | |
| MG169 E. coli codon | | 275 | MG169-9 | nucleotide |
| optimized genes | | | | |
| MG169 E. coli codon | | 276 | MG169-10 | nucleotide |
| optimized genes | | | | |
| MG169 E. coli codon | | 277 | MG169-11 | nucleotide |
| optimized genes | | | | |
| MG170 Strep tagged genes | | 278 | Nstrep-MG170-1 | nucleotide |
| MG170 Strep tagged genes | | 279 | Nstrep-MG170-2 | nucleotide |
| MG170 Strep tagged genes | | 280 | Nstrep-MG170-3 | nucleotide |
| MG170 Strep tagged genes | | 281 | Nstrep-MG170-4 | nucleotide |
| MG170 Strep tagged genes | | 282 | Nstrep-MG170-5 | nucleotide |
| MG170 Strep tagged genes | | 283 | Nstrep-MG170-6 | nucleotide |
| MG170 Strep tagged genes | | 284 | Nstrep-MG170-7 | nucleotide |
| MG170 Strep tagged genes | | 285 | Nstrep-MG170-8 | nucleotide |
| MG170 Strep tagged genes | | 286 | Nstrep-MG170-9 | nucleotide |
| MG170 Strep tagged genes | | 287 | Nstrep-MG170-10 | nucleotide |
| MG170 E. coli codon | | 288 | MG170-1 | nucleotide |
| optimized genes | | | | |
| MG170 E. coli codon | | 289 | MG170-2 | nucleotide |
| optimized genes | | | | |
| MG170 E. coli codon | | 290 | MG170-3 | nucleotide |
| optimized genes | | | | |
| MG170 E. coli codon | | 291 | MG170-4 | nucleotide |
| optimized genes | | | | |
| MG170 E. coli codon | | 292 | MG170-5 | nucleotide |
| optimized genes | | | | |
| MG170 E. coli codon | | 293 | MG170-6 | nucleotide |
| optimized genes | | | | |
| MG170 E. coli codon | | 294 | MG170-7 | nucleotide |
| optimized genes | | | | |
| MG170 E. coli codon | | 295 | MG170-8 | nucleotide |
| optimized genes | | | | |
| MG170 E. coli codon | | 296 | MG170-9 | nucleotide |
| optimized genes | | | | |
| MG170 E. coli codon | | 297 | MG170-10 | nucleotide |
| optimized genes | | | | |
| MG172 Strep tagged genes | | 298 | Nstrep-MG172-1 | nucleotide |
| MG172 Strep tagged genes | | 299 | Nstrep-MG172-2 | nucleotide |
| MG172 Strep tagged genes | | 300 | Nstrep-MG172-3 | nucleotide |
| MG172 Strep tagged genes | | 301 | Nstrep-MG172-4 | nucleotide |
| MG172 Strep tagged genes | | 302 | Nstrep-MG172-5 | nucleotide |
| MG172 E. coli codon | | 303 | MG172-1 | nucleotide |
| optimized genes | | | | |
| MG172 E. coli codon | | 304 | MG172-2 | nucleotide |
| optimized genes | | | | |
| MG172 E. coli codon | | 305 | MG172-3 | nucleotide |
| optimized genes | | | | |
| MG172 E. coli codon | | 306 | MG172-4 | nucleotide |
| optimized genes | | | | |
| MG172 E. coli codon | | 307 | MG172-5 | nucleotide |
| optimized genes | | | | |
| MG154 Strep tagged genes | | 308 | Nstrep-MG154-1 | nucleotide |
| MG154 Strep tagged genes | | 309 | Nstrep-MG154-2 | nucleotide |
| MG155 Strep tagged genes | | 310 | Nstrep-MG155-1 | nucleotide |
| MG155 Strep tagged genes | | 311 | Nstrep-MG155-2 | nucleotide |
| MG155 Strep tagged genes | | 312 | Nstrep-MG155-3 | nucleotide |
| MG156 Strep tagged genes | | 313 | Nstrep-MG156-1 | nucleotide |
| MG156 Strep tagged genes | | 314 | Nstrep-MG156-2 | nucleotide |
| MG157 Strep tagged genes | | 315 | Nstrep-MG157-1 | nucleotide |
| MG157 Strep tagged genes | | 316 | Nstrep-MG157-2 | nucleotide |
| MG157 Strep tagged genes | | 317 | Nstrep-MG157-3 | nucleotide |
| MG157 Strep tagged genes | | 318 | Nstrep-MG157-4 | nucleotide |
| MG157 Strep tagged genes | | 319 | Nstrep-MG157-5 | nucleotide |
| MG158 Strep tagged genes | | 320 | Nstrep-MG158-1 | nucleotide |
| MG159 Strep tagged genes | | 321 | Nstrep-MG159-1 | nucleotide |
| MG159 Strep tagged genes | | 322 | Nstrep-MG159-2 | nucleotide |
| MG159 Strep tagged genes | | 323 | Nstrep-MG159-3 | nucleotide |
| MG154 E. coli codon | | 324 | MG154-1 | nucleotide |
| optimized genes | | | | |
| MG154 E. coli codon | | 325 | MG154-2 | nucleotide |
| optimized genes | | | | |
| MG155 E. coli codon | | 326 | MG155-1 | nucleotide |
| optimized genes | | | | |
| MG155 E. coli codon | | 327 | MG155-2 | nucleotide |
| optimized genes | | | | |
| MG155 E. coli codon | | 328 | MG155-3 | nucleotide |
| optimized genes | | | | |
| MG156 E. coli codon | | 329 | MG156-1 | nucleotide |
| optimized genes | | | | |
| MG156 E. coli codon | | 330 | MG156-2 | nucleotide |
| optimized genes | | | | |
| MG157 E. coli codon | | 331 | MG157-1 | nucleotide |
| optimized genes | | | | |
| MG157 E. coli codon | | 332 | MG157-2 | nucleotide |
| optimized genes | | | | |
| MG157 E. coli codon | | 333 | MG157-3 | nucleotide |
| optimized genes | | | | |
| MG157 E. coli codon | | 334 | MG157-4 | nucleotide |
| optimized genes | | | | |
| MG157 E. coli codon | | 335 | MG157-5 | nucleotide |
| optimized genes | | | | |
| MG158 E. coli codon | | 336 | MG158-1 | nucleotide |
| optimized genes | | | | |
| MG159 E. coli codon | | 337 | MG159-1 | nucleotide |
| optimized genes | | | | |
| MG159 E. coli codon | | 338 | MG159-2 | nucleotide |
| optimized genes | | | | |
| MG159 E. coli codon | | 339 | MG159-3 | nucleotide |
| optimized genes | | | | |
| MG154 ncRNA | | 340 | MG154-1_ncRNA | nucleotide |
| MG154 ncRNA | | 341 | MG154-2_ncRNA | nucleotide |
| MG155 ncRNA | | 342 | MG155-1_ncRNA | nucleotide |
| MG155 ncRNA | | 343 | MG155-2_ncRNA | nucleotide |
| MG155 ncRNA | | 344 | MG155-3_ncRNA | nucleotide |
| MG156 ncRNA | | 345 | MG156-1_ncRNA | nucleotide |
| MG156 ncRNA | | 346 | MG156-2_ncRNA | nucleotide |
| MG157 ncRNA | | 347 | MG157-1_ncRNA | nucleotide |
| MG157 ncRNA | | 348 | MG157-2_ncRNA | nucleotide |
| MG157 ncRNA | | 349 | MG157-3_ncRNA | nucleotide |
| MG157 ncRNA | | 350 | MG157-4_ncRNA | nucleotide |
| MG157 ncRNA | | 351 | MG157-5_ncRNA | nucleotide |
| MG158 ncRNA | | 352 | MG158-1_ncRNA | nucleotide |
| MG159 ncRNA | | 353 | MG159-1_ncRNA | nucleotide |
| MG159 ncRNA | | 354 | MG159-2_ncRNA | nucleotide |
| MG159 ncRNA | | 355 | MG159-3_ncRNA | nucleotide |
| MG151 TwinStrep tagged | | 356 | TwinStrep-MG151-80 | nucleotide |
| genes | | | | |
| MG151 TwinStrep tagged | | 357 | TwinStrep-MG151-81 | nucleotide |
| genes | | | | |
| MG151 TwinStrep tagged | | 358 | TwinStrep-MG151-82 | nucleotide |
| genes | | | | |
| MG151 TwinStrep tagged | | 359 | TwinStrep-MG151-83 | nucleotide |
| genes | | | | |
| MG151 TwinStrep tagged | | 360 | TwinStrep-MG151-84 | nucleotide |
| genes | | | | |
| MG151 TwinStrep tagged | | 361 | TwinStrep-MG151-85 | nucleotide |
| genes | | | | |
| MG151 TwinStrep tagged | | 362 | TwinStrep-MG151-86 | nucleotide |
| genes | | | | |
| MG151 Strep tagged genes | | 363 | Strep-MG151-87 | nucleotide |
| MG151 Strep tagged genes | | 364 | Strep-MG151-88 | nucleotide |
| MG151 Strep tagged genes | | 365 | Strep-MG151-89 | nucleotide |
| MG151 Strep tagged genes | | 366 | Strep-MG151-90 | nucleotide |
| MG151 Strep tagged genes | | 367 | Strep-MG151-91 | nucleotide |
| MG151 Strep tagged genes | | 368 | Strep-MG151-92 | nucleotide |
| MG151 Strep tagged genes | | 369 | Strep-MG151-93 | nucleotide |
| MG151 Strep tagged genes | | 370 | Strep-MG151-94 | nucleotide |
| MG151 Strep tagged genes | | 371 | Strep-MG151-95 | nucleotide |
| MG151 Strep tagged genes | | 372 | Strep-MG151-96 | nucleotide |
| MG151 Strep tagged genes | | 373 | Strep-MG151-97 | nucleotide |
| MG140 HA-His tagged genes | | 374 | MG140-1-HA-His | nucleotide |
| MG140 HA-His tagged genes | | 375 | MG140-3-HA-His | nucleotide |
| MG140 HA-His tagged genes | | 376 | MG140-4-HA-His | nucleotide |
| MG140 HA-His tagged genes | | 377 | MG140-5-HA-His | nucleotide |
| MG140 HA-His tagged genes | | 378 | MG140-6-HA-His | nucleotide |
| MG140 HA-His tagged genes | | 379 | MG140-7-HA-His | nucleotide |
| MG140 HA-His tagged genes | | 380 | MG140-8-HA-His | nucleotide |
| MG140 HA-His tagged genes | | 381 | MG140-10-HA-His | nucleotide |
| MG140 HA-His tagged genes | | 382 | MG140-13-HA-His | nucleotide |
| MG140 HA-His tagged genes | | 383 | MG140-14-HA-His | nucleotide |
| MG140 HA-His tagged genes | | 384 | MG140-45-HA-His | nucleotide |
| MG140 HA-His tagged genes | | 385 | MG140-46-HA-His | nucleotide |
| MG140 HA-His tagged genes | | 386 | MG140-47-HA-His | nucleotide |
| MG146 HA-His tagged genes | | 387 | MG146-1-HA-His | nucleotide |
| MG147 HA-His tagged genes | | 388 | MG147-1-HA-His | nucleotide |
| MG148 HA-His tagged genes | | 389 | MG148-1-HA-His | nucleotide |
| MG148 HA-His tagged genes | | 390 | MG148-2-HA-His | nucleotide |
| MG148 HA-His tagged genes | | 391 | MG148-3-HA-His | nucleotide |
| MG148 HA-His tagged genes | | 392 | MG148-4-HA-His | nucleotide |
| MG140 transposition proteins | | 393 | MG140-45 transposition protein | protein |
| MG140 transposition proteins | | 394 | MG140-46 transposition protein | protein |
| MG140 transposition proteins | | 395 | MG140-47 transposition protein | protein |
| MG140 transposition proteins | | 396 | MG140-48 transposition protein | protein |
| MG140 transposition proteins | | 397 | MG140-49 transposition protein | protein |
| MG140 transposition proteins | | 398 | MG140-50 transposition protein | protein |
| MG140 transposition proteins | | 399 | MG140-51 transposition protein | protein |
| MG140 transposition proteins | | 400 | MG140-52 transposition protein | protein |
| MG140 transposition proteins | | 401 | MG140-53 transposition protein | protein |
| MG146 transposition proteins | | 402 | MG146-1 transposition protein | protein |
| MG148 reverse transcriptase | | 403 | MG148-13 reverse transcriptase | protein |
| proteins | | | | |
| MG148 reverse transcriptase | | 404 | MG148-14 reverse transcriptase | protein |
| proteins | | | | |
| MG148 reverse transcriptase | | 405 | MG148-15 reverse transcriptase | protein |
| proteins | | | | |
| MG148 reverse transcriptase | | 406 | MG148-16 reverse transcriptase | protein |
| proteins | | | | |
| MG148 reverse transcriptase | | 407 | MG148-17 reverse transcriptase | protein |
| proteins | | | | |
| MG148 reverse transcriptase | | 408 | MG148-18 reverse transcriptase | protein |
| proteins | | | | |
| MG148 reverse transcriptase | | 409 | MG148-19 reverse transcriptase | protein |
| proteins | | | | |
| MG148 reverse transcriptase | | 410 | MG148-20 reverse transcriptase | protein |
| proteins | | | | |
| MG148 reverse transcriptase | | 411 | MG148-21 reverse transcriptase | protein |
| proteins | | | | |
| MG148 reverse transcriptase | | 412 | MG148-22 reverse transcriptase | protein |
| proteins | | | | |
| MG148 reverse transcriptase | | 413 | MG148-23 reverse transcriptase | protein |
| proteins | | | | |
| MG148 reverse transcriptase | | 414 | MG148-24 reverse transcriptase | protein |
| proteins | | | | |
| MG148 reverse transcriptase | | 415 | MG148-25 reverse transcriptase | protein |
| proteins | | | | |
| MG148 reverse transcriptase | | 416 | MG148-26 reverse transcriptase | protein |
| proteins | | | | |
| MG148 reverse transcriptase | | 417 | MG148-27 reverse transcriptase | protein |
| proteins | | | | |
| MG148 reverse transcriptase | | 418 | MG148-29 reverse transcriptase | protein |
| proteins | | | | |
| MG148 reverse transcriptase | | 419 | MG148-30 reverse transcriptase | protein |
| proteins | | | | |
| MG148 reverse transcriptase | | 420 | MG148-31 reverse transcriptase | protein |
| proteins | | | | |
| MG148 reverse transcriptase | | 421 | MG148-32 reverse transcriptase | protein |
| proteins | | | | |
| MG148 reverse transcriptase | | 422 | MG148-33 reverse transcriptase | protein |
| proteins | | | | |
| MG148 reverse transcriptase | | 423 | MG148-34 reverse transcriptase | protein |
| proteins | | | | |
| MG148 reverse transcriptase | | 424 | MG148-35 reverse transcriptase | protein |
| proteins | | | | |
| MG148 reverse transcriptase | | 425 | MG148-36 reverse transcriptase | protein |
| proteins | | | | |
| MG148 reverse transcriptase | | 426 | MG148-37 reverse transcriptase | protein |
| proteins | | | | |
| MG149 reverse transcriptase | | 427 | MG149-1 reverse transcriptase | protein |
| proteins | | | | |
| MG149 reverse transcriptase | | 428 | MG149-2 reverse transcriptase | protein |
| proteins | | | | |
| MG149 reverse transcriptase | | 429 | MG149-3 reverse transcriptase | protein |
| proteins | | | | |
| MG149 reverse transcriptase | | 430 | MG149-5 reverse transcriptase | protein |
| proteins | | | | |
| MG149 reverse transcriptase | | 431 | MG149-6 reverse transcriptase | protein |
| proteins | | | | |
| MG149 reverse transcriptase | | 432 | MG149-7 reverse transcriptase | protein |
| proteins | | | | |
| MG149 reverse transcriptase | | 433 | MG149-8 reverse transcriptase | protein |
| proteins | | | | |
| MG149 reverse transcriptase | | 434 | MG149-9 reverse transcriptase | protein |
| proteins | | | | |
| MG149 reverse transcriptase | | 435 | MG149-10 reverse transcriptase | protein |
| proteins | | | | |
| MG149 reverse transcriptase | | 436 | MG149-11 reverse transcriptase | protein |
| proteins | | | | |
| MG149 reverse transcriptase | | 437 | MG149-12 reverse transcriptase | protein |
| proteins | | | | |
| MG149 reverse transcriptase | | 438 | MG149-13 reverse transcriptase | protein |
| proteins | | | | |
| MG149 reverse transcriptase | | 439 | MG149-14 reverse transcriptase | protein |
| proteins | | | | |
| MG151 reverse transcriptase | | 440 | MG151-1 reverse transcriptase | protein |
| proteins | | | | |
| MG151 reverse transcriptase | | 441 | MG151-2 reverse transcriptase | protein |
| proteins | | | | |
| MG151 reverse transcriptase | | 442 | MG151-3 reverse transcriptase | protein |
| proteins | | | | |
| MG151 reverse transcriptase | | 443 | MG151-4 reverse transcriptase | protein |
| proteins | | | | |
| MG151 reverse transcriptase | | 444 | MG151-5 reverse transcriptase | protein |
| proteins | | | | |
| MG151 reverse transcriptase | | 445 | MG151-6 reverse transcriptase | protein |
| proteins | | | | |
| MG151 reverse transcriptase | | 446 | MG151-7 reverse transcriptase | protein |
| proteins | | | | |
| MG151 reverse transcriptase | | 447 | MG151-8 reverse transcriptase | protein |
| proteins | | | | |
| MG151 reverse transcriptase | | 448 | MG151-9 reverse transcriptase | protein |
| proteins | | | | |
| MG151 reverse transcriptase | | 449 | MG151-10 reverse transcriptase | protein |
| proteins | | | | |
| MG151 reverse transcriptase | | 450 | MG151-12 reverse transcriptase | protein |
| proteins | | | | |
| MG151 reverse transcriptase | | 451 | MG151-13 reverse transcriptase | protein |
| proteins | | | | |
| MG151 reverse transcriptase | | 452 | MG151-14 reverse transcriptase | protein |
| proteins | | | | |
| MG151 reverse transcriptase | | 453 | MG151-15 reverse transcriptase | protein |
| proteins | | | | |
| MG151 reverse transcriptase | | 454 | MG151-16 reverse transcriptase | protein |
| proteins | | | | |
| MG151 reverse transcriptase | | 455 | MG151-17 reverse transcriptase | protein |
| proteins | | | | |
| MG151 reverse transcriptase | | 456 | MG151-18 reverse transcriptase | protein |
| proteins | | | | |
| MG151 reverse transcriptase | | 457 | MG151-19 reverse transcriptase | protein |
| proteins | | | | |
| MG151 reverse transcriptase | | 458 | MG151-20 reverse transcriptase | protein |
| proteins | | | | |
| MG151 reverse transcriptase | | 459 | MG151-21 reverse transcriptase | protein |
| proteins | | | | |
| MG151 reverse transcriptase | | 460 | MG151-22 reverse transcriptase | protein |
| proteins | | | | |
| MG151 reverse transcriptase | | 461 | MG151-23 reverse transcriptase | protein |
| proteins | | | | |
| MG151 reverse transcriptase | | 462 | MG151-24 reverse transcriptase | protein |
| proteins | | | | |
| MG151 reverse transcriptase | | 463 | MG151-25 reverse transcriptase | protein |
| proteins | | | | |
| MG151 reverse transcriptase | | 464 | MG151-26 reverse transcriptase | protein |
| proteins | | | | |
| MG151 reverse transcriptase | | 465 | MG151-27 reverse transcriptase | protein |
| proteins | | | | |
| MG151 reverse transcriptase | | 466 | MG151-28 reverse transcriptase | protein |
| proteins | | | | |
| MG151 reverse transcriptase | | 467 | MG151-29 reverse transcriptase | protein |
| proteins | | | | |
| MG151 reverse transcriptase | | 468 | MG151-30 reverse transcriptase | protein |
| proteins | | | | |
| MG151 reverse transcriptase | | 469 | MG151-31 reverse transcriptase | protein |
| proteins | | | | |
| MG151 reverse transcriptase | | 470 | MG151-32 reverse transcriptase | protein |
| proteins | | | | |
| MG151 reverse transcriptase | | 471 | MG151-33 reverse transcriptase | protein |
| proteins | | | | |
| MG151 reverse transcriptase | | 472 | MG151-34 reverse transcriptase | protein |
| proteins | | | | |
| MG151 reverse transcriptase | | 473 | MG151-35 reverse transcriptase | protein |
| proteins | | | | |
| MG151 reverse transcriptase | | 474 | MG151-36 reverse transcriptase | protein |
| proteins | | | | |
| MG151 reverse transcriptase | | 475 | MG151-37 reverse transcriptase | protein |
| proteins | | | | |
| MG151 reverse transcriptase | | 476 | MG151-38 reverse transcriptase | protein |
| proteins | | | | |
| MG151 reverse transcriptase | | 477 | MG151-39 reverse transcriptase | protein |
| proteins | | | | |
| MG151 reverse transcriptase | | 478 | MG151-40 reverse transcriptase | protein |
| proteins | | | | |
| MG151 reverse transcriptase | | 479 | MG151-41 reverse transcriptase | protein |
| proteins | | | | |
| MG151 reverse transcriptase | | 480 | MG151-42 reverse transcriptase | protein |
| proteins | | | | |
| MG151 reverse transcriptase | | 481 | MG151-43 reverse transcriptase | protein |
| proteins | | | | |
| MG151 reverse transcriptase | | 482 | MG151-44 reverse transcriptase | protein |
| proteins | | | | |
| MG151 reverse transcriptase | | 483 | MG151-45 reverse transcriptase | protein |
| proteins | | | | |
| MG151 reverse transcriptase | | 484 | MG151-46 reverse transcriptase | protein |
| proteins | | | | |
| MG151 reverse transcriptase | | 485 | MG151-47 reverse transcriptase | protein |
| proteins | | | | |
| MG151 reverse transcriptase | | 486 | MG151-48 reverse transcriptase | protein |
| proteins | | | | |
| MG151 reverse transcriptase | | 487 | MG151-49 reverse transcriptase | protein |
| proteins | | | | |
| MG151 reverse transcriptase | | 488 | MG151-50 reverse transcriptase | protein |
| proteins | | | | |
| MG151 reverse transcriptase | | 489 | MG151-51 reverse transcriptase | protein |
| proteins | | | | |
| MG151 reverse transcriptase | | 490 | MG151-52 reverse transcriptase | protein |
| proteins | | | | |
| MG151 reverse transcriptase | | 491 | MG151-53 reverse transcriptase | protein |
| proteins | | | | |
| MG151 reverse transcriptase | | 492 | MG151-54 reverse transcriptase | protein |
| proteins | | | | |
| MG151 reverse transcriptase | | 493 | MG151-55 reverse transcriptase | protein |
| proteins | | | | |
| MG151 reverse transcriptase | | 494 | MG151-56 reverse transcriptase | protein |
| proteins | | | | |
| MG151 reverse transcriptase | | 495 | MG151-57 reverse transcriptase | protein |
| proteins | | | | |
| MG151 reverse transcriptase | | 496 | MG151-58 reverse transcriptase | protein |
| proteins | | | | |
| MG151 reverse transcriptase | | 497 | MG151-59 reverse transcriptase | protein |
| proteins | | | | |
| MG151 reverse transcriptase | | 498 | MG151-60 reverse transcriptase | protein |
| proteins | | | | |
| MG151 reverse transcriptase | | 499 | MG151-61 reverse transcriptase | protein |
| proteins | | | | |
| MG151 reverse transcriptase | | 500 | MG151-62 reverse transcriptase | protein |
| proteins | | | | |
| MG151 reverse transcriptase | | 501 | MG151-63 reverse transcriptase | protein |
| proteins | | | | |
| MG151 reverse transcriptase | | 502 | MG151-64 reverse transcriptase | protein |
| proteins | | | | |
| MG151 reverse transcriptase | | 503 | MG151-65 reverse transcriptase | protein |
| proteins | | | | |
| MG151 reverse transcriptase | | 504 | MG151-66 reverse transcriptase | protein |
| proteins | | | | |
| MG151 reverse transcriptase | | 505 | MG151-67 reverse transcriptase | protein |
| proteins | | | | |
| MG151 reverse transcriptase | | 506 | MG151-68 reverse transcriptase | protein |
| proteins | | | | |
| MG151 reverse transcriptase | | 507 | MG151-69 reverse transcriptase | protein |
| proteins | | | | |
| MG151 reverse transcriptase | | 508 | MG151-70 reverse transcriptase | protein |
| proteins | | | | |
| MG151 reverse transcriptase | | 509 | MG151-71 reverse transcriptase | protein |
| proteins | | | | |
| MG151 reverse transcriptase | | 510 | MG151-72 reverse transcriptase | protein |
| proteins | | | | |
| MG151 reverse transcriptase | | 511 | MG151-73 reverse transcriptase | protein |
| proteins | | | | |
| MG151 reverse transcriptase | | 512 | MG151-74 reverse transcriptase | protein |
| proteins | | | | |
| MG151 reverse transcriptase | | 513 | MG151-75 reverse transcriptase | protein |
| proteins | | | | |
| MG151 reverse transcriptase | | 514 | MG151-76 reverse transcriptase | protein |
| proteins | | | | |
| MG151 reverse transcriptase | | 515 | MG151-77 reverse transcriptase | protein |
| proteins | | | | |
| MG151 reverse transcriptase | | 516 | MG151-78 reverse transcriptase | protein |
| proteins | | | | |
| MG151 reverse transcriptase | | 517 | MG151-79 reverse transcriptase | protein |
| proteins | | | | |
| MG151 reverse transcriptase | | 518 | MG151-80 reverse transcriptase | protein |
| proteins | | | | |
| MG151 reverse transcriptase | | 519 | MG151-81 reverse transcriptase | protein |
| proteins | | | | |
| MG151 reverse transcriptase | | 520 | MG151-82 reverse transcriptase | protein |
| proteins | | | | |
| MG151 reverse transcriptase | | 521 | MG151-83 reverse transcriptase | protein |
| proteins | | | | |
| MG151 reverse transcriptase | | 522 | MG151-84 reverse transcriptase | protein |
| proteins | | | | |
| MG151 reverse transcriptase | | 523 | MG151-85 reverse transcriptase | protein |
| proteins | | | | |
| MG151 reverse transcriptase | | 524 | MG151-87 reverse transcriptase | protein |
| proteins | | | | |
| MG151 reverse transcriptase | | 525 | MG151-88 reverse transcriptase | protein |
| proteins | | | | |
| MG151 reverse transcriptase | | 526 | MG151-89 reverse transcriptase | protein |
| proteins | | | | |
| MG151 reverse transcriptase | | 527 | MG151-90 reverse transcriptase | protein |
| proteins | | | | |
| MG151 reverse transcriptase | | 528 | MG151-91 reverse transcriptase | protein |
| proteins | | | | |
| MG151 reverse transcriptase | | 529 | MG151-92 reverse transcriptase | protein |
| proteins | | | | |
| MG151 reverse transcriptase | | 530 | MG151-93 reverse transcriptase | protein |
| proteins | | | | |
| MG151 reverse transcriptase | | 531 | MG151-94 reverse transcriptase | protein |
| proteins | | | | |
| MG151 reverse transcriptase | | 532 | MG151-95 reverse transcriptase | protein |
| proteins | | | | |
| MG151 reverse transcriptase | | 533 | MG151-96 reverse transcriptase | protein |
| proteins | | | | |
| MG151 reverse transcriptase | | 534 | MG151-97 reverse transcriptase | protein |
| proteins | | | | |
| MG151 reverse transcriptase | | 535 | MG151-98 reverse transcriptase | protein |
| proteins | | | | |
| MG151 reverse transcriptase | | 536 | MG151-99 reverse transcriptase | protein |
| proteins | | | | |
| MG151 reverse transcriptase | | 537 | MG151-100 reverse transcriptase | protein |
| proteins | | | | |
| MG151 reverse transcriptase | | 538 | MG151-101 reverse transcriptase | protein |
| proteins | | | | |
| MG151 reverse transcriptase | | 539 | MG151-102 reverse transcriptase | protein |
| proteins | | | | |
| MG151 reverse transcriptase | | 540 | MG151-103 reverse transcriptase | protein |
| proteins | | | | |
| MG151 reverse transcriptase | | 541 | MG151-104 reverse transcriptase | protein |
| proteins | | | | |
| MG151 reverse transcriptase | | 542 | MG151-105 reverse transcriptase | protein |
| proteins | | | | |
| MG151 reverse transcriptase | | 543 | MG151-106 reverse transcriptase | protein |
| proteins | | | | |
| MG151 reverse transcriptase | | 544 | MG151-107 reverse transcriptase | protein |
| proteins | | | | |
| MG151 reverse transcriptase | | 545 | MG151-108 reverse transcriptase | protein |
| proteins | | | | |
| MG151 reverse transcriptase | | 546 | MG151-109 reverse transcriptase | protein |
| proteins | | | | |
| MG151 reverse transcriptase | | 547 | MG151-110 reverse transcriptase | protein |
| proteins | | | | |
| MG151 reverse transcriptase | | 548 | MG151-111 reverse transcriptase | protein |
| proteins | | | | |
| MG151 reverse transcriptase | | 549 | MG151-112 reverse transcriptase | protein |
| proteins | | | | |
| MG151 reverse transcriptase | | 550 | MG151-113 reverse transcriptase | protein |
| proteins | | | | |
| MG151 reverse transcriptase | | 551 | MG151-114 reverse transcriptase | protein |
| proteins | | | | |
| MG151 reverse transcriptase | | 552 | MG151-115 reverse transcriptase | protein |
| proteins | | | | |
| MG151 reverse transcriptase | | 553 | MG151-116 reverse transcriptase | protein |
| proteins | | | | |
| MG151 reverse transcriptase | | 554 | MG151-117 reverse transcriptase | protein |
| proteins | | | | |
| MG153 reverse transcriptase | | 555 | MG153-1 reverse transcriptase | protein |
| proteins | | | | |
| MG153 reverse transcriptase | | 556 | MG153-2 reverse transcriptase | protein |
| proteins | | | | |
| MG153 reverse transcriptase | | 557 | MG153-3 reverse transcriptase | protein |
| proteins | | | | |
| MG153 reverse transcriptase | | 558 | MG153-4 reverse transcriptase | protein |
| proteins | | | | |
| MG153 reverse transcriptase | | 559 | MG153-5 reverse transcriptase | protein |
| proteins | | | | |
| MG153 reverse transcriptase | | 560 | MG153-6 reverse transcriptase | protein |
| proteins | | | | |
| MG153 reverse transcriptase | | 561 | MG153-7 reverse transcriptase | protein |
| proteins | | | | |
| MG153 reverse transcriptase | | 562 | MG153-8 reverse transcriptase | protein |
| proteins | | | | |
| MG153 reverse transcriptase | | 563 | MG153-9 reverse transcriptase | protein |
| proteins | | | | |
| MG153 reverse transcriptase | | 564 | MG153-10 reverse transcriptase | protein |
| proteins | | | | |
| MG153 reverse transcriptase | | 565 | MG153-11 reverse transcriptase | protein |
| proteins | | | | |
| MG153 reverse transcriptase | | 566 | MG153-12 reverse transcriptase | protein |
| proteins | | | | |
| MG153 reverse transcriptase | | 567 | MG153-13 reverse transcriptase | protein |
| proteins | | | | |
| MG153 reverse transcriptase | | 568 | MG153-14 reverse transcriptase | protein |
| proteins | | | | |
| MG153 reverse transcriptase | | 569 | MG153-15 reverse transcriptase | protein |
| proteins | | | | |
| MG153 reverse transcriptase | | 570 | MG153-16 reverse transcriptase | protein |
| proteins | | | | |
| MG153 reverse transcriptase | | 571 | MG153-17 reverse transcriptase | protein |
| proteins | | | | |
| MG153 reverse transcriptase | | 572 | MG153-18 reverse transcriptase | protein |
| proteins | | | | |
| MG153 reverse transcriptase | | 573 | MG153-19 reverse transcriptase | protein |
| proteins | | | | |
| MG153 reverse transcriptase | | 574 | MG153-20 reverse transcriptase | protein |
| proteins | | | | |
| MG153 reverse transcriptase | | 575 | MG153-21 reverse transcriptase | protein |
| proteins | | | | |
| MG153 reverse transcriptase | | 576 | MG153-25 reverse transcriptase | protein |
| proteins | | | | |
| MG153 reverse transcriptase | | 577 | MG153-26 reverse transcriptase | protein |
| proteins | | | | |
| MG153 reverse transcriptase | | 578 | MG153-27 reverse transcriptase | protein |
| proteins | | | | |
| MG153 reverse transcriptase | | 579 | MG153-28 reverse transcriptase | protein |
| proteins | | | | |
| MG153 reverse transcriptase | | 580 | MG153-29 reverse transcriptase | protein |
| proteins | | | | |
| MG153 reverse transcriptase | | 581 | MG153-30 reverse transcriptase | protein |
| proteins | | | | |
| MG153 reverse transcriptase | | 582 | MG153-31 reverse transcriptase | protein |
| proteins | | | | |
| MG153 reverse transcriptase | | 583 | MG153-32 reverse transcriptase | protein |
| proteins | | | | |
| MG153 reverse transcriptase | | 584 | MG153-33 reverse transcriptase | protein |
| proteins | | | | |
| MG153 reverse transcriptase | | 585 | MG153-34 reverse transcriptase | protein |
| proteins | | | | |
| MG153 reverse transcriptase | | 586 | MG153-35 reverse transcriptase | protein |
| proteins | | | | |
| MG153 reverse transcriptase | | 587 | MG153-36 reverse transcriptase | protein |
| proteins | | | | |
| MG153 reverse transcriptase | | 588 | MG153-37 reverse transcriptase | protein |
| proteins | | | | |
| MG153 reverse transcriptase | | 589 | MG153-38 reverse transcriptase | protein |
| proteins | | | | |
| MG153 reverse transcriptase | | 590 | MG153-39 reverse transcriptase | protein |
| proteins | | | | |
| MG153 reverse transcriptase | | 591 | MG153-40 reverse transcriptase | protein |
| proteins | | | | |
| MG153 reverse transcriptase | | 592 | MG153-41 reverse transcriptase | protein |
| proteins | | | | |
| MG153 reverse transcriptase | | 593 | MG153-42 reverse transcriptase | protein |
| proteins | | | | |
| MG153 reverse transcriptase | | 594 | MG153-43 reverse transcriptase | protein |
| proteins | | | | |
| MG153 reverse transcriptase | | 595 | MG153-44 reverse transcriptase | protein |
| proteins | | | | |
| MG153 reverse transcriptase | | 596 | MG153-45 reverse transcriptase | protein |
| proteins | | | | |
| MG153 reverse transcriptase | | 597 | MG153-46 reverse transcriptase | protein |
| proteins | | | | |
| MG153 reverse transcriptase | | 598 | MG153-47 reverse transcriptase | protein |
| proteins | | | | |
| MG153 reverse transcriptase | | 599 | MG153-48 reverse transcriptase | protein |
| proteins | | | | |
| MG153 reverse transcriptase | | 600 | MG153-49 reverse transcriptase | protein |
| proteins | | | | |
| MG153 reverse transcriptase | | 601 | MG153-50 reverse transcriptase | protein |
| proteins | | | | |
| MG153 reverse transcriptase | | 602 | MG153-51 reverse transcriptase | protein |
| proteins | | | | |
| MG153 reverse transcriptase | | 603 | MG153-52 reverse transcriptase | protein |
| proteins | | | | |
| MG153 reverse transcriptase | | 604 | MG153-53 reverse transcriptase | protein |
| proteins | | | | |
| MG153 reverse transcriptase | | 605 | MG153-54 reverse transcriptase | protein |
| proteins | | | | |
| MG153 reverse transcriptase | | 606 | MG153-55 reverse transcriptase | protein |
| proteins | | | | |
| MG153 reverse transcriptase | | 607 | MG153-56 reverse transcriptase | protein |
| proteins | | | | |
| MG153 reverse transcriptase | | 608 | MG153-57 reverse transcriptase | protein |
| proteins | | | | |
| MG154 reverse transcriptase | | 609 | MG154-1 reverse transcriptase | protein |
| proteins | | | | |
| MG154 reverse transcriptase | | 610 | MG154-2 reverse transcriptase | protein |
| proteins | | | | |
| MG155 reverse transcriptase | | 611 | MG155-1 reverse transcriptase | protein |
| proteins | | | | |
| MG155 reverse transcriptase | | 612 | MG155-2 reverse transcriptase | protein |
| proteins | | | | |
| MG155 reverse transcriptase | | 613 | MG155-3 reverse transcriptase | protein |
| proteins | | | | |
| MG155 reverse transcriptase | | 614 | MG155-4 reverse transcriptase | protein |
| proteins | | | | |
| MG155 reverse transcriptase | | 615 | MG155-5 reverse transcriptase | protein |
| proteins | | | | |
| MG156 reverse transcriptase | | 616 | MG156-1 reverse transcriptase | protein |
| proteins | | | | |
| MG156 reverse transcriptase | | 617 | MG156-2 reverse transcriptase | protein |
| proteins | | | | |
| MG157 reverse transcriptase | | 618 | MG157-1 reverse transcriptase | protein |
| proteins | | | | |
| MG157 reverse transcriptase | | 619 | MG157-2 reverse transcriptase | protein |
| proteins | | | | |
| MG157 reverse transcriptase | | 620 | MG157-3 reverse transcriptase | protein |
| proteins | | | | |
| MG157 reverse transcriptase | | 621 | MG157-4 reverse transcriptase | protein |
| proteins | | | | |
| MG157 reverse transcriptase | | 622 | MG157-5 reverse transcriptase | protein |
| proteins | | | | |
| MG158 reverse transcriptase | | 623 | MG158-1 reverse transcriptase | protein |
| proteins | | | | |
| MG159 reverse transcriptase | | 624 | MG159-1 reverse transcriptase | protein |
| proteins | | | | |
| MG159 reverse transcriptase | | 625 | MG159-2 reverse transcriptase | protein |
| proteins | | | | |
| MG159 reverse transcriptase | | 626 | MG159-3 reverse transcriptase | protein |
| proteins | | | | |
| MG160 reverse transcriptase | | 627 | MG160-1 reverse transcriptase | protein |
| proteins | | | | |
| MG160 reverse transcriptase | | 628 | MG160-2 reverse transcriptase | protein |
| proteins | | | | |
| MG160 reverse transcriptase | | 629 | MG160-3 reverse transcriptase | protein |
| proteins | | | | |
| MG160 reverse transcriptase | | 630 | MG160-4 reverse transcriptase | protein |
| proteins | | | | |
| MG160 reverse transcriptase | | 631 | MG160-5 reverse transcriptase | protein |
| proteins | | | | |
| MG160 reverse transcriptase | | 632 | MG160-8 reverse transcriptase | protein |
| proteins | | | | |
| MG160 reverse transcriptase | | 633 | MG160-6 reverse transcriptase | protein |
| proteins | | | | |
| MG160 reverse transcriptase | | 634 | MG160-9 reverse transcriptase | protein |
| proteins | | | | |
| MG160 reverse transcriptase | | 635 | MG160-10 reverse transcriptase | protein |
| proteins | | | | |
| MG160 reverse transcriptase | | 636 | MG160-11 reverse transcriptase | protein |
| proteins | | | | |
| MG160 reverse transcriptase | | 637 | MG160-12 reverse transcriptase | protein |
| proteins | | | | |
| MG160 reverse transcriptase | | 638 | MG160-13 reverse transcriptase | protein |
| proteins | | | | |
| MG160 reverse transcriptase | | 639 | MG160-14 reverse transcriptase | protein |
| proteins | | | | |
| MG160 reverse transcriptase | | 640 | MG160-15 reverse transcriptase | protein |
| proteins | | | | |
| MG160 reverse transcriptase | | 641 | MG160-16 reverse transcriptase | protein |
| proteins | | | | |
| MG160 reverse transcriptase | | 642 | MG160-17 reverse transcriptase | protein |
| proteins | | | | |
| MG160 reverse transcriptase | | 643 | MG160-18 reverse transcriptase | protein |
| proteins | | | | |
| MG160 reverse transcriptase | | 644 | MG160-19 reverse transcriptase | protein |
| proteins | | | | |
| MG160 reverse transcriptase | | 645 | MG160-20 reverse transcriptase | protein |
| proteins | | | | |
| MG160 reverse transcriptase | | 646 | MG160-21 reverse transcriptase | protein |
| proteins | | | | |
| MG160 reverse transcriptase | | 647 | MG160-22 reverse transcriptase | protein |
| proteins | | | | |
| MG160 reverse transcriptase | | 648 | MG160-23 reverse transcriptase | protein |
| proteins | | | | |
| MG160 reverse transcriptase | | 649 | MG160-24 reverse transcriptase | protein |
| proteins | | | | |
| MG160 reverse transcriptase | | 650 | MG160-25 reverse transcriptase | protein |
| proteins | | | | |
| MG160 reverse transcriptase | | 651 | MG160-26 reverse transcriptase | protein |
| proteins | | | | |
| MG160 reverse transcriptase | | 652 | MG160-27 reverse transcriptase | protein |
| proteins | | | | |
| MG160 reverse transcriptase | | 653 | MG160-28 reverse transcriptase | protein |
| proteins | | | | |
| MG160 reverse transcriptase | | 654 | MG160-29 reverse transcriptase | protein |
| proteins | | | | |
| MG160 reverse transcriptase | | 655 | MG160-30 reverse transcriptase | protein |
| proteins | | | | |
| MG160 reverse transcriptase | | 656 | MG160-31 reverse transcriptase | protein |
| proteins | | | | |
| MG160 reverse transcriptase | | 657 | MG160-32 reverse transcriptase | protein |
| proteins | | | | |
| MG160 reverse transcriptase | | 658 | MG160-33 reverse transcriptase | protein |
| proteins | | | | |
| MG160 reverse transcriptase | | 659 | MG160-34 reverse transcriptase | protein |
| proteins | | | | |
| MG160 reverse transcriptase | | 660 | MG160-35 reverse transcriptase | protein |
| proteins | | | | |
| MG160 reverse transcriptase | | 661 | MG160-36 reverse transcriptase | protein |
| proteins | | | | |
| MG160 reverse transcriptase | | 662 | MG160-37 reverse transcriptase | protein |
| proteins | | | | |
| MG160 reverse transcriptase | | 663 | MG160-38 reverse transcriptase | protein |
| proteins | | | | |
| MG160 reverse transcriptase | | 664 | MG160-39 reverse transcriptase | protein |
| proteins | | | | |
| MG160 reverse transcriptase | | 665 | MG160-40 reverse transcriptase | protein |
| proteins | | | | |
| MG160 reverse transcriptase | | 666 | MG160-41 reverse transcriptase | protein |
| proteins | | | | |
| MG160 reverse transcriptase | | 667 | MG160-42 reverse transcriptase | protein |
| proteins | | | | |
| MG160 reverse transcriptase | | 668 | MG160-43 reverse transcriptase | protein |
| proteins | | | | |
| MG160 reverse transcriptase | | 669 | MG160-44 reverse transcriptase | protein |
| proteins | | | | |
| MG160 reverse transcriptase | | 670 | MG160-45 reverse transcriptase | protein |
| proteins | | | | |
| MG160 reverse transcriptase | | 671 | MG160-46 reverse transcriptase | protein |
| proteins | | | | |
| MG160 reverse transcriptase | | 672 | MG160-47 reverse transcriptase | protein |
| proteins | | | | |
| MG160 reverse transcriptase | | 673 | MG160-48 reverse transcriptase | protein |
| proteins | | | | |
| MG163 reverse transcriptase | | 674 | MG163-1 reverse transcriptase | protein |
| proteins | | | | |
| MG163 reverse transcriptase | | 675 | MG163-2 reverse transcriptase | protein |
| proteins | | | | |
| MG163 reverse transcriptase | | 676 | MG163-3 reverse transcriptase | protein |
| proteins | | | | |
| MG163 reverse transcriptase | | 677 | MG163-4 reverse transcriptase | protein |
| proteins | | | | |
| MG163 reverse transcriptase | | 678 | MG163-5 reverse transcriptase | protein |
| proteins | | | | |
| MG164 reverse transcriptase | | 679 | MG164-1 reverse transcriptase | protein |
| proteins | | | | |
| MG164 reverse transcriptase | | 680 | MG164-2 reverse transcriptase | protein |
| proteins | | | | |
| MG164 reverse transcriptase | | 681 | MG164-3 reverse transcriptase | protein |
| proteins | | | | |
| MG164 reverse transcriptase | | 682 | MG164-4 reverse transcriptase | protein |
| proteins | | | | |
| MG164 reverse transcriptase | | 683 | MG164-5 reverse transcriptase | protein |
| proteins | | | | |
| MG165 reverse transcriptase | | 684 | MG165-1 reverse transcriptase | protein |
| proteins | | | | |
| MG165 reverse transcriptase | | 685 | MG165-2 reverse transcriptase | protein |
| proteins | | | | |
| MG165 reverse transcriptase | | 686 | MG165-3 reverse transcriptase | protein |
| proteins | | | | |
| MG165 reverse transcriptase | | 687 | MG165-4 reverse transcriptase | protein |
| proteins | | | | |
| MG165 reverse transcriptase | | 688 | MG165-5 reverse transcriptase | protein |
| proteins | | | | |
| MG165 reverse transcriptase | | 689 | MG165-6 reverse transcriptase | protein |
| proteins | | | | |
| MG165 reverse transcriptase | | 690 | MG165-7 reverse transcriptase | protein |
| proteins | | | | |
| MG165 reverse transcriptase | | 691 | MG165-8 reverse transcriptase | protein |
| proteins | | | | |
| MG165 reverse transcriptase | | 692 | MG165-9 reverse transcriptase | protein |
| proteins | | | | |
| MG166 reverse transcriptase | | 693 | MG166-1 reverse transcriptase | protein |
| proteins | | | | |
| MG166 reverse transcriptase | | 694 | MG166-2 reverse transcriptase | protein |
| proteins | | | | |
| MG166 reverse transcriptase | | 695 | MG166-3 reverse transcriptase | protein |
| proteins | | | | |
| MG166 reverse transcriptase | | 696 | MG166-4 reverse transcriptase | protein |
| proteins | | | | |
| MG166 reverse transcriptase | | 697 | MG166-5 reverse transcriptase | protein |
| proteins | | | | |
| MG167 reverse transcriptase | | 698 | MG167-1 reverse transcriptase | protein |
| proteins | | | | |
| MG167 reverse transcriptase | | 699 | MG167-2 reverse transcriptase | protein |
| proteins | | | | |
| MG167 reverse transcriptase | | 700 | MG167-3 reverse transcriptase | protein |
| proteins | | | | |
| MG167 reverse transcriptase | | 701 | MG167-4 reverse transcriptase | protein |
| proteins | | | | |
| MG167 reverse transcriptase | | 702 | MG167-5 reverse transcriptase | protein |
| proteins | | | | |
| MG168 reverse transcriptase | | 703 | MG168-1 reverse transcriptase | protein |
| proteins | | | | |
| MG168 reverse transcriptase | | 704 | MG168-2 reverse transcriptase | protein |
| proteins | | | | |
| MG168 reverse transcriptase | | 705 | MG168-3 reverse transcriptase | protein |
| proteins | | | | |
| MG168 reverse transcriptase | | 706 | MG168-4 reverse transcriptase | protein |
| proteins | | | | |
| MG168 reverse transcriptase | | 707 | MG168-5 reverse transcriptase | protein |
| proteins | | | | |
| MG169 reverse transcriptase | | 708 | MG169-1 reverse transcriptase | protein |
| proteins | | | | |
| MG169 reverse transcriptase | | 709 | MG169-2 reverse transcriptase | protein |
| proteins | | | | |
| MG169 reverse transcriptase | | 710 | MG169-3 reverse transcriptase | protein |
| proteins | | | | |
| MG169 reverse transcriptase | | 711 | MG169-4 reverse transcriptase | protein |
| proteins | | | | |
| MG169 reverse transcriptase | | 712 | MG169-5 reverse transcriptase | protein |
| proteins | | | | |
| MG169 reverse transcriptase | | 713 | MG169-6 reverse transcriptase | protein |
| proteins | | | | |
| MG169 reverse transcriptase | | 714 | MG169-7 reverse transcriptase | protein |
| proteins | | | | |
| MG169 reverse transcriptase | | 715 | MG169-8 reverse transcriptase | protein |
| proteins | | | | |
| MG169 reverse transcriptase | | 716 | MG169-9 reverse transcriptase | protein |
| proteins | | | | |
| MG169 reverse transcriptase | | 717 | MG169-10 reverse transcriptase | protein |
| proteins | | | | |
| MG169 reverse transcriptase | | 718 | MG169-11 reverse transcriptase | protein |
| proteins | | | | |
| MG170 reverse transcriptase | | 719 | MG170-1 reverse transcriptase | protein |
| proteins | | | | |
| MG170 reverse transcriptase | | 720 | MG170-2 reverse transcriptase | protein |
| proteins | | | | |
| MG170 reverse transcriptase | | 721 | MG170-3 reverse transcriptase | protein |
| proteins | | | | |
| MG170 reverse transcriptase | | 722 | MG170-4 reverse transcriptase | protein |
| proteins | | | | |
| MG170 reverse transcriptase | | 723 | MG170-5 reverse transcriptase | protein |
| proteins | | | | |
| MG170 reverse transcriptase | | 724 | MG170-6 reverse transcriptase | protein |
| proteins | | | | |
| MG170 reverse transcriptase | | 725 | MG170-7 reverse transcriptase | protein |
| proteins | | | | |
| MG170 reverse transcriptase | | 726 | MG170-8 reverse transcriptase | protein |
| proteins | | | | |
| MG170 reverse transcriptase | | 727 | MG170-9 reverse transcriptase | protein |
| proteins | | | | |
| MG170 reverse transcriptase | | 728 | MG170-10 reverse transcriptase | protein |
| proteins | | | | |
| MG172 reverse transcriptase | | 729 | MG172-1 reverse transcriptase | protein |
| proteins | | | | |
| MG172 reverse transcriptase | | 730 | MG172-2 reverse transcriptase | protein |
| proteins | | | | |
| MG172 reverse transcriptase | | 731 | MG172-3 reverse transcriptase | protein |
| proteins | | | | |
| MG172 reverse transcriptase | | 732 | MG172-4 reverse transcriptase | protein |
| proteins | | | | |
| MG172 reverse transcriptase | | 733 | MG172-5 reverse transcriptase | protein |
| proteins | | | | |
| MG173 reverse transcriptase | | 734 | MG173-1 reverse transcriptase | protein |
| proteins | | | | |
| MG173 reverse transcriptase | | 735 | MG173-2 reverse transcriptase | protein |
| proteins | | | | |
| PS modified primers | | 736 | PS-modified DNA primer #1, PS bond | nucleotide |
| | | | denoted by * | |
| PS modified primers | | 737 | PS-modified DNA primer #2, PS bond | nucleotide |
| | | | denoted by * | |
| PS modified primers | | 738 | PS-modified DNA primer #3, PS bond | nucleotide |
| | | | denoted by * | |
| Tagman probe for qPCR | | 739 | Tagman probe for 542 bp amplicon | nucleotide |
| MG153 RT MCP fusions | | 740 | FH-MCP-MG153-5 | nucleotide |
| MG153 RT MCP fusions | | 741 | FH-MCP-MG153-6 | nucleotide |
| MG153 RT MCP fusions | | 742 | FH-MCP-MG153-18 | nucleotide |
| MG153 RT MCP fusions | | 743 | FH-MCP-MG153-20 | nucleotide |
| MG153 RT MCP fusions | | 744 | FH-MCP-MG153-29 | nucleotide |
| MG153 RT MCP fusions | | 745 | FH-MCP-MG153-30 | nucleotide |
| MG153 RT MCP fusions | | 746 | FH-MCP-MG153-31 | nucleotide |
| MG153 RT MCP fusions | | 747 | FH-MCP-MG153-33 | nucleotide |
| MG153 RT MCP fusions | | 748 | FH-MCP-MG153-34 | nucleotide |
| MG153 RT MCP fusions | | 749 | FH-MCP-MG153-35 | nucleotide |
| MG153 RT MCP fusions | | 750 | FH-MCP-MG153-36 | nucleotide |
| MG153 RT MCP fusions | | 751 | FH-MCP-MG153-37 | nucleotide |
| MG153 RT MCP fusions | | 752 | FH-MCP-MG153-45 | nucleotide |
| MG153 RT MCP fusions | | 753 | FH-MCP-MG153-51 | nucleotide |
| MG153 RT MCP fusions | | 754 | FH-MCP-MG153-53 | nucleotide |
| MG153 RT MCP fusions | | 755 | FH-MCP-MG153-54 | nucleotide |
| MG153 RT MCP fusions | | 756 | FH-MCP-MG153-57 | nucleotide |
| MG165 RT MCP fusions | | 757 | FH-MCP-MG165-1 | nucleotide |
| MG165 RT MCP fusions | | 758 | FH-MCP-MG165-5 | nucleotide |
| MG167 RT MCP fusions | | 759 | FH-MCP-MG167-1 | nucleotide |
| MG167 RT MCP fusions | | 760 | FH-MCP-MG167-4 | nucleotide |
| MG140 UTR | | 761 | MG140-54 5′ UTR | nucleotide |
| MG140 UTR | | 762 | MG140-54 3′ UTR | nucleotide |
| MG140 UTR | | 763 | MG140-55 5′ UTR | nucleotide |
| MG140 UTR | | 764 | MG140-55 3′ UTR | nucleotide |
| MG140 UTR | | 765 | MG140-56 5′ UTR | nucleotide |
| MG140 UTR | | 766 | MG140-56 3′ UTR | nucleotide |
| MG140 UTR | | 767 | MG140-1 5′ UTR | nucleotide |
| MG140 UTR | | 768 | MG140-1 3′ UTR | nucleotide |
| MG140 UTR | | 769 | MG140-3 5′ UTR | nucleotide |
| MG140 UTR | | 770 | MG140-3 3′ UTR | nucleotide |
| MG140 UTR | | 771 | MG140-4 5′ UTR | nucleotide |
| MG140 UTR | | 772 | MG140-4 3′ UTR | nucleotide |
| MG140 UTR | | 773 | MG140-5 5′ UTR | nucleotide |
| MG140 UTR | | 774 | MG140-5 3′ UTR | nucleotide |
| MG140 UTR | | 775 | MG140-6 5′ UTR | nucleotide |
| MG140 UTR | | 776 | MG140-6 3′ UTR | nucleotide |
| MG140 UTR | | 777 | MG140-7 5′ UTR | nucleotide |
| MG140 UTR | | 778 | MG140-7 3′ UTR | nucleotide |
| MG140 UTR | | 779 | MG140-8 5′ UTR | nucleotide |
| MG140 UTR | | 780 | MG140-8 3′ UTR | nucleotide |
| MG140 UTR | | 781 | MG140-10 5′ UTR | nucleotide |
| MG140 UTR | | 782 | MG140-10 3′ UTR | nucleotide |
| MG140 UTR | | 783 | MG140-13 5′ UTR | nucleotide |
| MG140 UTR | | 784 | MG140-13 3′ UTR | nucleotide |
| MG140 UTR | | 785 | MG140-14 5′ UTR | nucleotide |
| MG140 UTR | | 786 | MG140-14 3′ UTR | nucleotide |
| MG140 UTR | | 787 | MG140-45 5′ UTR | nucleotide |
| MG140 UTR | | 788 | MG140-45 3′ UTR | nucleotide |
| MG140 UTR | | 789 | MG140-46 5′ UTR | nucleotide |
| MG140 UTR | | 790 | MG140-46 3′ UTR | nucleotide |
| MG140 UTR | | 791 | MG140-47 5′ UTR | nucleotide |
| MG140 UTR | | 792 | MG140-47 3′ UTR | nucleotide |
| MG140 UTR | | 793 | MG140-54 5′ UTR | nucleotide |
| MG140 UTR | | 794 | MG140-54 3′ UTR | nucleotide |
| MG140 UTR | | 795 | MG140-55 5′ UTR | nucleotide |
| MG140 UTR | | 796 | MG140-55 3′ UTR | nucleotide |
| MG140 UTR | | 797 | MG140-56 5′ UTR | nucleotide |
| MG140 UTR | | 798 | MG140-56 3′ UTR | nucleotide |
| MG140 reverse transcriptase | | 799 | MG140-54 reverse transcriptase | protein |
| proteins | | | | |
| MG140 reverse transcriptase | | 800 | MG140-55 reverse transcriptase | protein |
| proteins | | | | |
| MG140 reverse transcriptase | | 801 | MG140-56 reverse transcriptase | protein |
| proteins | | | | |
| MG140 reverse transcriptase | | 802 | MG140-54 reverse transcriptase | protein |
| proteins | | | | |
| MG140 reverse transcriptase | | 803 | MG140-55 reverse transcriptase | protein |
| proteins | | | | |
| MG140 reverse transcriptase | | 804 | MG140-56 reverse transcriptase | protein |
| proteins | | | | |
| MG140 reverse transcriptase | | 805 | MG140-57 reverse transcriptase | protein |
| proteins | | | | |
| MG140 reverse transcriptase | | 806 | MG140-58 reverse transcriptase | protein |
| proteins | | | | |
| MG140 reverse transcriptase | | 807 | MG140-59 reverse transcriptase | protein |
| proteins | | | | |
| MG140 reverse transcriptase | | 808 | MG140-60 reverse transcriptase | protein |
| proteins | | | | |
| MG140 reverse transcriptase | | 809 | MG140-61 reverse transcriptase | protein |
| proteins | | | | |
| MG140 reverse transcriptase | | 810 | MG140-62 reverse transcriptase | protein |
| proteins | | | | |
| MG140 reverse transcriptase | | 811 | MG140-63 reverse transcriptase | protein |
| proteins | | | | |
| MG140 reverse transcriptase | | 812 | MG140-64 reverse transcriptase | protein |
| proteins | | | | |
| MG140 reverse transcriptase | | 813 | MG140-65 reverse transcriptase | protein |
| proteins | | | | |
| MG140 reverse transcriptase | | 814 | MG140-66 reverse transcriptase | protein |
| proteins | | | | |
| MG140 reverse transcriptase | | 815 | MG140-67 reverse transcriptase | protein |
| proteins | | | | |
| MG140 reverse transcriptase | | 816 | MG140-68 reverse transcriptase | protein |
| proteins | | | | |
| MG140 reverse transcriptase | | 817 | MG140-69 reverse transcriptase | protein |
| proteins | | | | |
| MG140 reverse transcriptase | | 818 | MG140-70 reverse transcriptase | protein |
| proteins | | | | |
| MG140 reverse transcriptase | | 819 | MG140-71 reverse transcriptase | protein |
| proteins | | | | |
| MG140 reverse transcriptase | | 820 | MG140-72 reverse transcriptase | protein |
| proteins | | | | |
| MG140 reverse transcriptase | | 821 | MG140-73 reverse transcriptase | protein |
| proteins | | | | |
| MG140 reverse transcriptase | | 822 | MG140-74 reverse transcriptase | protein |
| proteins | | | | |
| MG140 reverse transcriptase | | 823 | MG140-75 reverse transcriptase | protein |
| proteins | | | | |
| MG140 reverse transcriptase | | 824 | MG140-76 reverse transcriptase | protein |
| proteins | | | | |
| MG140 reverse transcriptase | | 825 | MG140-77 reverse transcriptase | protein |
| proteins | | | | |
| MG140 reverse transcriptase | | 826 | MG140-78 reverse transcriptase | protein |
| proteins | | | | |
| MG140 reverse transcriptase | | 827 | MG140-79 reverse transcriptase | protein |
| proteins | | | | |
| MG140 reverse transcriptase | | 828 | MG140-80 reverse transcriptase | protein |
| proteins | | | | |
| MG140 reverse transcriptase | | 829 | MG140-81 reverse transcriptase | protein |
| proteins | | | | |
| MG140 reverse transcriptase | | 830 | MG140-82 reverse transcriptase | protein |
| proteins | | | | |
| MG140 reverse transcriptase | | 831 | MG140-83 reverse transcriptase | protein |
| proteins | | | | |
| MG140 reverse transcriptase | | 832 | MG140-84 reverse transcriptase | protein |
| proteins | | | | |
| MG140 reverse transcriptase | | 833 | MG140-85 reverse transcriptase | protein |
| proteins | | | | |
| MG140 reverse transcriptase | | 834 | MG140-86 reverse transcriptase | protein |
| proteins | | | | |
| MG140 reverse transcriptase | | 835 | MG140-87 reverse transcriptase | protein |
| proteins | | | | |
| MG140 reverse transcriptase | | 836 | MG140-88 reverse transcriptase | protein |
| proteins | | | | |
| MG140 reverse transcriptase | | 837 | MG140-89 reverse transcriptase | protein |
| proteins | | | | |
| MG140 reverse transcriptase | | 838 | MG140-90 reverse transcriptase | protein |
| proteins | | | | |
| MG140 reverse transcriptase | | 839 | MG140-91 reverse transcriptase | protein |
| proteins | | | | |
| MG140 reverse transcriptase | | 840 | MG140-92 reverse transcriptase | protein |
| proteins | | | | |
| MG140 reverse transcriptase | | 841 | MG140-93 reverse transcriptase | protein |
| proteins | | | | |
| MG140 reverse transcriptase | | 842 | MG140-94 reverse transcriptase | protein |
| proteins | | | | |
| MG140 reverse transcriptase | | 843 | MG140-95 reverse transcriptase | protein |
| proteins | | | | |
| MG140 reverse transcriptase | | 844 | MG140-96 reverse transcriptase | protein |
| proteins | | | | |
| MG140 reverse transcriptase | | 845 | MG140-97 reverse transcriptase | protein |
| proteins | | | | |
| MG140 reverse transcriptase | | 846 | MG140-98 reverse transcriptase | protein |
| proteins | | | | |
| MG140 reverse transcriptase | | 847 | MG140-99 reverse transcriptase | protein |
| proteins | | | | |
| MG140 reverse transcriptase | | 848 | MG140-100 reverse transcriptase | protein |
| proteins | | | | |
| MG140 reverse transcriptase | | 849 | MG140-101 reverse transcriptase | protein |
| proteins | | | | |
| MG140 reverse transcriptase | | 850 | MG140-102 reverse transcriptase | protein |
| proteins | | | | |
| MG140 reverse transcriptase | | 851 | MG140-103 reverse transcriptase | protein |
| proteins | | | | |
| MG140 reverse transcriptase | | 852 | MG140-104 reverse transcriptase | protein |
| proteins | | | | |
| MG140 reverse transcriptase | | 853 | MG140-105 reverse transcriptase | protein |
| proteins | | | | |
| MG140 reverse transcriptase | | 854 | MG140-106 reverse transcriptase | protein |
| proteins | | | | |
| MG140 reverse transcriptase | | 855 | MG140-107 reverse transcriptase | protein |
| proteins | | | | |
| MG140 reverse transcriptase | | 856 | MG140-108 reverse transcriptase | protein |
| proteins | | | | |
| MG140 reverse transcriptase | | 857 | MG140-109 reverse transcriptase | protein |
| proteins | | | | |
| MG140 reverse transcriptase | | 858 | MG140-110 reverse transcriptase | protein |
| proteins | | | | |
| MG140 reverse transcriptase | | 859 | MG140-111 reverse transcriptase | protein |
| proteins | | | | |
| MG140 reverse transcriptase | | 860 | MG140-112 reverse transcriptase | protein |
| proteins | | | | |
| MG140 reverse transcriptase | | 861 | MG140-113 reverse transcriptase | protein |
| proteins | | | | |
| MG140 reverse transcriptase | | 862 | MG140-114 reverse transcriptase | protein |
| proteins | | | | |
| MG140 reverse transcriptase | | 863 | MG140-115 reverse transcriptase | protein |
| proteins | | | | |
| MG140 reverse transcriptase | | 864 | MG140-116 reverse transcriptase | protein |
| proteins | | | | |
| MG140 reverse transcriptase | | 865 | MG140-117 reverse transcriptase | protein |
| proteins | | | | |
| MG140 reverse transcriptase | | 866 | MG140-118 reverse transcriptase | protein |
| proteins | | | | |
| MG140 reverse transcriptase | | 867 | MG140-119 reverse transcriptase | protein |
| proteins | | | | |
| MG140 reverse transcriptase | | 868 | MG140-120 reverse transcriptase | protein |
| proteins | | | | |
| MG140 reverse transcriptase | | 869 | MG140-121 reverse transcriptase | protein |
| proteins | | | | |
| MG140 reverse transcriptase | | 870 | MG140-122 reverse transcriptase | protein |
| proteins | | | | |
| MG140 reverse transcriptase | | 871 | MG140-123 reverse transcriptase | protein |
| proteins | | | | |
| MG140 reverse transcriptase | | 872 | MG140-124 reverse transcriptase | protein |
| proteins | | | | |
| MG140 reverse transcriptase | | 873 | MG140-125 reverse transcriptase | protein |
| proteins | | | | |
| MG140 reverse transcriptase | | 874 | MG140-126 reverse transcriptase | protein |
| proteins | | | | |
| MG140 reverse transcriptase | | 875 | MG140-127 reverse transcriptase | protein |
| proteins | | | | |
| MG140 reverse transcriptase | | 876 | MG140-128 reverse transcriptase | protein |
| proteins | | | | |
| MG140 reverse transcriptase | | 877 | MG140-129 reverse transcriptase | protein |
| proteins | | | | |
| MG140 reverse transcriptase | | 878 | MG140-130 reverse transcriptase | protein |
| proteins | | | | |
| MG140 reverse transcriptase | | 879 | MG140-131 reverse transcriptase | protein |
| proteins | | | | |
| MG140 reverse transcriptase | | 880 | MG140-132 reverse transcriptase | protein |
| proteins | | | | |
| MG140 reverse transcriptase | | 881 | MG140-133 reverse transcriptase | protein |
| proteins | | | | |
| MG140 reverse transcriptase | | 882 | MG140-134 reverse transcriptase | protein |
| proteins | | | | |
| MG140 reverse transcriptase | | 883 | MG140-135 reverse transcriptase | protein |
| proteins | | | | |
| MG140 reverse transcriptase | | 884 | MG140-136 reverse transcriptase | protein |
| proteins | | | | |
| MG140 reverse transcriptase | | 885 | MG140-137 reverse transcriptase | protein |
| proteins | | | | |
| MG140 reverse transcriptase | | 886 | MG140-138 reverse transcriptase | protein |
| proteins | | | | |
| MG140 reverse transcriptase | | 887 | MG140-139 reverse transcriptase | protein |
| proteins | | | | |
| MG140 reverse transcriptase | | 888 | MG140-140 reverse transcriptase | protein |
| proteins | | | | |
| MG140 reverse transcriptase | | 889 | MG140-141 reverse transcriptase | protein |
| proteins | | | | |
| MG140 reverse transcriptase | | 890 | MG140-142 reverse transcriptase | protein |
| proteins | | | | |
| MG140 reverse transcriptase | | 891 | MG140-143 reverse transcriptase | protein |
| proteins | | | | |
| MG140 reverse transcriptase | | 892 | MG140-144 reverse transcriptase | protein |
| proteins | | | | |
| MG140 reverse transcriptase | | 893 | MG140-145 reverse transcriptase | protein |
| proteins | | | | |
| MG140 reverse transcriptase | | 894 | MG140-146 reverse transcriptase | protein |
| proteins | | | | |
| MG146 transposition proteins | | 895 | MG146-2 transposition protein | protein |

### EMBODIMENTS

The following embodiments are not intended to be limiting in any way.
Embodiment 1. An engineered retrotransposase system, comprising:
   (a) an RNA comprising a heterologous engineered cargo nucleotide sequence, wherein said cargo nucleotide sequence is configured to interact with a retrotransposase; and
   (b) a retrotransposase, wherein:
said retrotransposase is configured to transpose said cargo nucleotide sequence to a target nucleic acid locus; and said retrotransposase is derived from an uncultivated microorganism.
Embodiment 2. The engineered retrotransposase system of embodiment Embodiment 1, wherein said retrotransposase comprises a sequence having at least 75% sequence identity to any one of SEQ ID NOs: 1-29, 393-735, or 799-895.
Embodiment 3. The engineered retrotransposase system of embodiment Embodiment 1 or embodiment Embodiment 2, wherein said retrotransposase comprises a reverse transcriptase domain.
Embodiment 4. The engineered retrotransposase system of any one of embodiments Embodiment 1 to Embodiment 3, wherein said retrotransposase further comprises one or more zinc finger domains.
Embodiment 5. The engineered retrotransposase system of any one of embodiments Embodiment 1 to Embodiment 4, wherein said retrotransposase further comprises an endonuclease domain.
Embodiment 6. The engineered retrotransposase system of any one of embodiments Embodiment 1 to Embodiment 5, wherein said retrotransposase has less than 80% sequence identity to a documented retrotransposase.
Embodiment 7. The engineered retrotransposase system of any one of embodiments Embodiment 1 to Embodiment 6, wherein said cargo nucleotide sequence is flanked by a 3′ untranslated region (UTR) and a 5′ untranslated region (UTR).
Embodiment 8. The engineered retrotransposase system of any one of embodiments Embodiment 1 to Embodiment 7, wherein said retrotransposase is configured to transpose said cargo nucleotide sequence via a ribonucleic acid polynucleotide intermediate.
Embodiment 9. The engineered retrotransposase system of any one of embodiments Embodiment 1 to Embodiment 8, wherein said retrotransposase comprises one or more nuclear localization sequences (NLSs) proximal to an N- or C-terminus of said retrotransposase.
Embodiment 10. The engineered retrotransposase system of any one of embodiments Embodiment 1 to Embodiment 9, wherein said NLS comprises a sequence at least 80% identical to a sequence selected from the group consisting of SEQ ID NO: 896-911.
Embodiment 11. The engineered retrotransposase system of any one of embodiments Embodiment 1 to Embodiment 10, wherein said sequence identity is determined by a BLASTP, CLUSTALW, MUSCLE, MAFFT, or CLUSTALW with the parameters of the Smith-Waterman homology search algorithm.
Embodiment 12. The engineered retrotransposase system of embodiment Embodiment 11, wherein said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.
Embodiment 13. An engineered retrotransposase system, comprising:
   (a) an RNA comprising a heterologous engineered cargo nucleotide sequence, wherein said cargo nucleotide sequence is configured to interact with a retrotransposase; and
   (b) a retrotransposase, wherein:
said retrotransposase is configured to transpose said cargo nucleotide sequence to a target nucleic acid locus; and said retrotransposase comprises a sequence having at least 75% sequence identity to any one of SEQ ID NOs: 1-29, 393-735, or 799-895.
Embodiment 14. The engineered retrotransposase system of embodiment Embodiment 13, wherein said retrotransposase is derived from an uncultivated microorganism.
Embodiment 15. The engineered retrotransposase system of embodiment Embodiment 13 or embodiment Embodiment 14, wherein said retrotransposase comprises a reverse transcriptase domain.
Embodiment 16. The engineered retrotransposase system of any one of embodiments Embodiment 13 to Embodiment 15, wherein said retrotransposase further comprises one or more zinc finger domains.
Embodiment 17. The engineered retrotransposase system of any one of embodiments Embodiment 13 to Embodiment 16, wherein said retrotransposase further comprises an endonuclease domain.
Embodiment 18. The engineered retrotransposase system of any one of embodiments Embodiment 13 to Embodiment 17, wherein said retrotransposase has less than 80% sequence identity to a documented retrotransposase.
Embodiment 19. The engineered retrotransposase system of any one of embodiments Embodiment 13 to Embodiment 18, wherein said cargo nucleotide sequence is flanked by a 3′ untranslated region (UTR) and a 5′ untranslated region (UTR).
Embodiment 20. The engineered retrotransposase system of any one of embodiments Embodiment 13 to Embodiment 19, wherein said retrotransposase is configured to transpose said cargo nucleotide sequence via a ribonucleic acid polynucleotide intermediate.
Embodiment 21. The engineered retrotransposase system of any one of embodiments Embodiment 13 to Embodiment 20, wherein said sequence identity is determined by a BLASTP, CLUSTALW, MUSCLE, MAFFT, or CLUSTALW with the parameters of the Smith-Waterman homology search algorithm.
Embodiment 22. The engineered retrotransposase system of embodiment Embodiment 21, wherein said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.
Embodiment 23. A deoxyribonucleic acid polynucleotide encoding said engineered retrotransposase system of any one of embodiments Embodiment 1 to Embodiment 22.
Embodiment 24. A nucleic acid comprising an engineered nucleic acid sequence optimized for expression in an organism, wherein said nucleic acid encodes a retrotransposase, and wherein said retrotransposase is derived from an uncultivated microorganism, wherein said organism is not said uncultivated microorganism.
Embodiment 25. The nucleic acid of embodiment Embodiment 24, wherein said retrotransposase comprises a variant having at least 75% sequence identity to any one of SEQ ID NOs: 1-29, 393-735, or 799-895.
Embodiment 26. The nucleic acid of embodiment Embodiment 24 or embodiment Embodiment 25, wherein said retrotransposase comprises a sequence encoding one or more nuclear localization sequences (NLSs) proximal to an N- or C-terminus of said retrotransposase.
Embodiment 27. The nucleic acid of embodiment Embodiment 26, wherein said NLS comprises a sequence selected from SEQ ID NOs: 896-911.
Embodiment 28. The nucleic acid of embodiment Embodiment 26 or Embodiment 27, wherein said NLS comprises SEQ ID NO: 897.
Embodiment 29. The nucleic acid of embodiment Embodiment 28, wherein said NLS is proximal to said N-terminus of said retrotransposase.
Embodiment 30. The nucleic acid of embodiment Embodiment 26 or Embodiment 27, wherein said NLS comprises SEQ ID NO: 896.
Embodiment 31. The nucleic acid of embodiment Embodiment 30, wherein said NLS is proximal to said C-terminus of said retrotransposase.
Embodiment 32. The nucleic acid of any one of embodiments Embodiment 24 to Embodiment 31, wherein said organism is prokaryotic, bacterial, eukaryotic, fungal, plant, mammalian, rodent, or human.
Embodiment 33. A vector comprising said nucleic acid of any one of embodiments Embodiment 24 to Embodiment 32.
Embodiment 34. The vector of embodiment Embodiment 33, further comprising a nucleic acid encoding a cargo nucleotide sequence configured to form a complex with said retrotransposase.
Embodiment 35. The vector of embodiment Embodiment 33 or embodiment Embodiment 34, wherein said vector is a plasmid, a minicircle, a CELiD, an adeno-associated virus (AAV) derived virion, or a lentivirus.
Embodiment 36. A cell comprising said vector of any one of any one of embodiments Embodiment 33 to Embodiment 35.
Embodiment 37. A method of manufacturing a retrotransposase, comprising cultivating said cell of embodiment Embodiment 36.
Embodiment 38. A method for disrupting, binding, nicking, cleaving, marking, or modifying a double-stranded deoxyribonucleic acid polynucleotide comprising a target nucleic acid locus, comprising:
   (a) contacting said double-stranded deoxyribonucleic acid polynucleotide comprising said target nucleic acid locus with a retrotransposase configured to transpose a cargo nucleotide sequence to said target nucleic acid locus; and
   (b) wherein said retrotransposase comprises a sequence having at least 75% sequence identity to any one of SEQ ID NOs: 1-29, 393-735, or 799-895.
Embodiment 39. The method of embodiment Embodiment 38, wherein said retrotransposase is derived from an uncultivated microorganism.
Embodiment 40. The engineered retrotransposase system of embodiment Embodiment 38 or embodiment Embodiment 39, wherein said retrotransposase comprises a reverse transcriptase domain.
Embodiment 41. The engineered retrotransposase system of any one of embodiments Embodiment 38 to Embodiment 40, wherein said retrotransposase further comprises one or more zinc finger domains.
Embodiment 42. The engineered retrotransposase system of any one of embodiments Embodiment 38 to Embodiment 41, wherein said retrotransposase further comprises an endonuclease domain.
Embodiment 43. The method of any one of embodiments Embodiment 38 to Embodiment 42, wherein said retrotransposase has less than 80% sequence identity to a documented retrotransposase.
Embodiment 44. The engineered retrotransposase system of any one of embodiments Embodiment 38 to Embodiment 43, wherein said cargo nucleotide sequence is flanked by a 3′ untranslated region (UTR) and a 5′ untranslated region (UTR).
Embodiment 45. The method of any one of embodiments Embodiment 38 to Embodiment 44, wherein said double-stranded deoxyribonucleic acid polynucleotide is transposed via a ribonucleic acid polynucleotide intermediate.
Embodiment 46. The method of any one of embodiments Embodiment 38 to Embodiment 45, wherein said double-stranded deoxyribonucleic acid polynucleotide is a eukaryotic, plant, fungal, mammalian, rodent, or human double-stranded deoxyribonucleic acid polynucleotide.
Embodiment 47. A method of disrupting or modifying a target nucleic acid locus, said method comprising delivering to said target nucleic acid locus said engineered retrotransposase system of any one of embodiments Embodiment 1 to Embodiment 22, wherein said retrotransposase is configured to transpose a cargo nucleotide sequence to said target nucleic acid locus, and wherein said complex is configured such that upon binding of said complex to said target nucleic acid locus, said complex modifies said target nucleic acid locus.
Embodiment 48. The method of embodiment Embodiment 47, wherein modifying said target nucleic acid locus comprises binding, nicking, cleaving, marking, modifying, or transposing said target nucleic acid locus.
Embodiment 49. The method of embodiment Embodiment 47 to Embodiment 48, wherein said target nucleic acid locus comprises deoxyribonucleic acid (DNA).
Embodiment 50. The method of embodiment Embodiment 49, wherein said target nucleic acid locus comprises genomic DNA, viral DNA, or bacterial DNA.
Embodiment 51. The method of any one of embodiments Embodiment 47 to Embodiment 50, wherein said target nucleic acid locus is in vitro.
Embodiment 52. The method of any one of embodiments Embodiment 47 to Embodiment 50, wherein said target nucleic acid locus is within a cell.
Embodiment 53. The method of embodiment Embodiment 52, wherein said cell is a prokaryotic cell, a bacterial cell, a eukaryotic cell, a fungal cell, a plant cell, an animal cell, a mammalian cell, a rodent cell, a primate cell, a human cell, or a primary cell.
Embodiment 54. The method of embodiment Embodiment 52 or Embodiment 53, wherein said cell is a primary cell.
Embodiment 55. The method of embodiment Embodiment 54, wherein said primary cell is a T cell.
Embodiment 56. The method of embodiment Embodiment 54, wherein said primary cell is a hematopoietic stem cell (HSC).
Embodiment 57. The method of any one of embodiments Embodiment 47-Embodiment 56, wherein delivering said engineered retrotransposase system to said target nucleic acid locus comprises delivering the nucleic acid of any one of embodiments Embodiment 24-Embodiment 32 or the vector of any of embodiments Embodiment 33-Embodiment 35.
Embodiment 58. The method of any one of embodiments Embodiment 47-Embodiment 57, wherein delivering said engineered retrotransposase system to said target nucleic acid locus comprises delivering a nucleic acid comprising an open reading frame encoding said retrotransposase.
Embodiment 59. The method of embodiment Embodiment 58, wherein said nucleic acid comprises a promoter to which said open reading frame encoding said retrotransposase is operably linked.
Embodiment 60. The method of any one of embodiments Embodiment 47 to Embodiment 59, wherein delivering said engineered retrotransposase system to said target nucleic acid locus comprises delivering a capped mRNA containing said open reading frame encoding said retrotransposase.
Embodiment 61. The method of any one of embodiments Embodiment 47 to Embodiment 60, wherein delivering said engineered retrotransposase system to said target nucleic acid locus comprises delivering a translated polypeptide.
Embodiment 62. The method of any one of embodiments Embodiment 47 to Embodiment 61, wherein said retrotransposase does not induce a break at or proximal to said target nucleic acid locus.
Embodiment 63. A host cell comprising an open reading frame encoding a heterologous retrotransposase having at least 75% sequence identity to any one of SEQ ID NOs: 1-29, 393-735, or 799-895 or a variant thereof.
Embodiment 64. The host cell of embodiment Embodiment 63, wherein said host cell is an E. coli cell.
Embodiment 65. The host cell of embodiment Embodiment 64, wherein said E. coli cell is a λDE3 lysogen or said E. coli cell is a BL21(DE3) strain.
Embodiment 66. The host cell of embodiment Embodiment 64 or embodiment Embodiment 65, wherein said E. coli cell has an ompT ion genotype.
Embodiment 67. The host cell of any one of embodiments Embodiment 63 to Embodiment 66, wherein said open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof.
Embodiment 68. The host cell of any one of embodiments Embodiment 63 to Embodiment 67, wherein said open reading frame comprises a sequence encoding an affinity tag linked in-frame to a sequence encoding said retrotransposase.
Embodiment 69. The host cell of embodiment Embodiment 68, wherein said affinity tag is an immobilized metal affinity chromatography (IMAC) tag.
Embodiment 70. The host cell of embodiment Embodiment 69, wherein said IMAC tag is a polyhistidine tag.
Embodiment 71. The host cell of embodiment Embodiment 68, wherein said affinity tag is a myc tag, a human influenza hemagglutinin (HA) tag, a maltose binding protein (MBP) tag, a glutathione S-transferase (GST) tag, a streptavidin tag, a FLAG tag, or any combination thereof.
Embodiment 72. The host cell of any one of embodiments Embodiment 68 to Embodiment 71, wherein said affinity tag is linked in-frame to said sequence encoding said retrotransposase via a linker sequence encoding a protease cleavage site.
Embodiment 73. The host cell of embodiment Embodiment 72, wherein said protease cleavage site is a tobacco etch virus (TEV) protease cleavage site, a PreScission® protease cleavage site, a Thrombin cleavage site, a Factor Xa cleavage site, an enterokinase cleavage site, or any combination thereof.
Embodiment 74. The host cell of any one of embodiments Embodiment 63 to Embodiment 73, wherein said open reading frame is codon-optimized for expression in said host cell.
Embodiment 75. The host cell of any one of embodiments Embodiment 63 to Embodiment 74, wherein said open reading frame is provided on a vector.
Embodiment 76. The host cell of any one of embodiments Embodiment 63 to Embodiment 74, wherein said open reading frame is integrated into a genome of said host cell.
Embodiment 77. A culture comprising the host cell of any one of embodiments Embodiment 63 to Embodiment 76 in compatible liquid medium.
Embodiment 78. A method of producing a retrotransposase, comprising cultivating the host cell of any one of embodiments Embodiment 63 to Embodiment 76 in compatible growth medium.
Embodiment 79. The method of embodiment Embodiment 78, further comprising inducing expression of said retrotransposase by addition of an additional chemical agent or an increased amount of a nutrient.
Embodiment 80. The method of embodiment Embodiment 79, wherein said additional chemical agent or increased amount of a nutrient comprises Isopropyl β-D-1-thiogalactopyranoside (IPTG) or additional amounts of lactose.
Embodiment 81. The method of any one of embodiments Embodiment 78 to Embodiment 80, further comprising isolating said host cell after said cultivation and lysing said host cell to produce a protein extract.
Embodiment 82. The method of embodiment Embodiment 81, further comprising subjecting said protein extract to IMAC, or ion-affinity chromatography.
Embodiment 83. The method of embodiment Embodiment 82, wherein said open reading frame comprises a sequence encoding an IMAC affinity tag linked in-frame to a sequence encoding said retrotransposase.
Embodiment 84. The method of embodiment Embodiment 83, wherein said IMAC affinity tag is linked in-frame to said sequence encoding said retrotransposase via a linker sequence encoding protease cleavage site.
Embodiment 85. The method of embodiment Embodiment 84, wherein said protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof.
Embodiment 86. The method of embodiment Embodiment 84 or embodiment Embodiment 85, further comprising cleaving said IMAC affinity tag by contacting a protease corresponding to said protease cleavage site to said retrotransposase.
Embodiment 87. The method of embodiment Embodiment 86, further comprising performing subtractive IMAC affinity chromatography to remove said affinity tag from a composition comprising said retrotransposase.
Embodiment 88. A method of disrupting a locus in a cell, comprising contacting to said cell
   a composition comprising:
      (a) a double-stranded nucleic acid comprising a heterologous engineered cargo nucleotide sequence, wherein said cargo nucleotide sequence is configured to interact with a retrotransposase; and
      (b) a retrotransposase, wherein:
   said retrotransposase is configured to transpose said cargo nucleotide sequence to a target nucleic acid locus;
      said retrotransposase comprises a sequence having at least 75% sequence identity to any one of SEQ ID NOs: 1-29, 393-735, or 799-895, or a variant thereof; and
      said retrotransposase has at least equivalent transposition activity to a documented retrotransposase in a cell.
Embodiment 89. The method of embodiment Embodiment 88, wherein said transposition activity is measured in vitro by introducing said retrotransposase to cells comprising said target nucleic acid locus and detecting transposition of said target nucleic acid locus in said cells.
Embodiment 90. The method of embodiment Embodiment 88 or embodiment Embodiment 89, wherein said composition comprises 20 pmoles or less of said retrotransposase.

1.-141. (canceled)

142. An engineered retrotransposase system, comprising:
(a) a ribonucleic acid (RNA) comprising a heterologous engineered cargo nucleotide sequence, wherein said RNA is configured to interact with a retrotransposase; and
(b) said retrotransposase, wherein:
(i) said retrotransposase is configured to transpose said heterologous engineered cargo nucleotide sequence to a target nucleic acid locus; and
(ii) said retrotransposase comprises a reverse transcriptase (RT) domain or an endonuclease domain, wherein said RT domain or said endonuclease domain comprises a sequence having at least 80% sequence identity to an RT domain or an endonuclease domain of any one of SEQ ID NOs: 3, 8, 822, 836, 1, 2, 4-7, 9-29, or 393-401.

143. The engineered retrotransposase system of claim 142, wherein said RT domain or said endonuclease domain of said retrotransposase comprises a sequence having at least 90% sequence identity to an RT domain or an endonuclease domain of SEQ ID NO: 3.

144. The engineered retrotransposase system of claim 142, wherein said RT domain or said endonuclease domain of said retrotransposase comprises a sequence having at least 90% sequence identity to an RT domain or an endonuclease domain of SEQ ID NO: 8.

145. The engineered retrotransposase system of claim 142, wherein said retrotransposase comprises an RT domain comprising a sequence having at least 90% sequence identity to an RT domain of SEQ ID NO: 822.

146. The engineered retrotransposase system of claim 142, wherein said retrotransposase comprises an RT domain comprising a sequence having at least 90% sequence identity to an RT domain of SEQ ID NO: 836.

147. The engineered retrotransposase system of claim 142, wherein said RNA comprises a sequence 5′ to said heterologous engineered cargo nucleotide sequence or a sequence 3′ to said heterologous engineered cargo nucleotide sequence, wherein said sequence 5′ to said heterologous engineered cargo nucleotide sequence or said sequence 3′ to said heterologous engineered cargo nucleotide sequence has at least 80% sequence identity to an RNA cognate of any one of SEQ ID NOs: 769, 770, 779, 780, 761-768, 771-778, 781-798, a complement thereof, or a reverse complement thereof.

148. The engineered retrotransposase system of claim 142, further comprising: (c) a double-stranded DNA sequence comprising said target nucleic acid locus.

149. The engineered retrotransposase system of claim 148, wherein said double-stranded DNA sequence comprises a 5′ recognition sequence configured to interact with said retrotransposase and a 3′ recognition sequence configured to interact with said retrotransposase,
wherein said 5′ recognition sequence comprises a GG nucleotide sequence, and
wherein said 3′ recognition sequence comprises a TGAC nucleotide sequence.

150. An engineered deoxyribonucleic acid (DNA) sequence encoding said engineered retrotransposase system of claim 142.

151. The engineered retrotransposase system of claim 142, wherein said RNA comprising said heterologous engineered cargo nucleotide and said retrotransposase are in a cell, and wherein said target nucleic acid locus is in said cell.

152. The engineered retrotransposase system of claim 151, wherein said cell is a eukaryotic cell.

153. A method for synthesizing complementary deoxyribonucleic acid (cDNA), comprising:
(a) providing a ribonucleic acid (RNA) molecule as a template for cDNA synthesis, wherein said RNA molecule comprises a heterologous engineered cargo nucleotide sequence,
(b) providing a nucleic acid primer to initiate cDNA synthesis from said RNA molecule; and
(c) synthesizing cDNA initiated by said nucleic acid primer using said template and a reverse transcriptase comprising a sequence having at least 80% sequence identity to a reverse transcriptase (RT) domain of any one of SEQ ID NOs: 3, 8, 822, 836, 1, 2, 4-7, 9-29, 393-401, or 427-439.

154. The method of claim 153, wherein said reverse transcriptase comprises a sequence having at least 90% sequence identity to an RT domain of SEQ ID NO: 3.

155. The method of claim 153, wherein said reverse transcriptase comprises a sequence having at least 90% sequence identity to an RT domain of SEQ ID NO: 8.

156. The method of claim 153, wherein said reverse transcriptase comprises a sequence having at least 90% sequence identity to SEQ ID NO: 822.

157. The method of claim 153, wherein said reverse transcriptase comprises a sequence having at least 90% sequence identity to SEQ ID NO: 836.

158. The method of claim 153, wherein said nucleic acid primer comprises an oligo(dT) sequence or a degenerate sequence of at least six oligonucleotides.

159. The method of claim 153, wherein said RNA molecule and said nucleic acid primer are provided in a cell, and wherein said synthesizing cDNA occurs in said cell.

160. The method of claim 159, wherein said cell is a eukaryotic cell.

161. A method for synthesizing complementary deoxyribonucleic acid (cDNA), comprising:
(a) providing a ribonucleic acid (RNA) molecule as a template for cDNA synthesis, wherein said RNA molecule comprises a heterologous engineered cargo nucleotide sequence,
(b) providing a nucleic acid primer to initiate cDNA synthesis from said RNA molecule; and
(c) synthesizing cDNA initiated by said primer oligonucleotide using said template and a reverse transcriptase comprising a sequence having at least 80% sequence identity to a reverse transcriptase (RT) domain of any one of SEQ ID NOs: 572, 602, 555-571, 573-601, or 603-728.
